(19) United States
(12) Patent Application Publication
(10) Pub. No.: US 2014/0045758 A1
(43) Pub. Date: Feb. 13, 2014

(54) Methods for Treating and Preventing Cardiac Dysfunction in Septic Shock

(71) Applicants: Ira J. Goldberg, New York, NY (US); Konstantinos Drosatos, Mineola, NY (US)

(72) Inventors: Ira J. Goldberg, New York, NY (US); Konstantinos Drosatos, Mineola, NY (US)

(73) Assignee: THE TRUSTEES OF COLUMBIA UNIVERSITY IN THE CITY OF NEW YORK, New York, NY (US)

(21) Appl. No.: 14/000,800

(22) PCT Filed: Feb. 21, 2012
(86) PCT No.: PCT/US12/25983

§ 371 Date: Oct. 25, 2013

Related U.S. Application Data
(60) Provisional application No. 61/445,002, filed on Feb. 21, 2011.

(51) Int. Cl. A61K 31/416; A61K 31/4439; A61K 38/17; A61K 31/739; A61K 31/7105; A61K 31/505

(52) U.S. Cl. CPC A61K 31/416; A61K 31/7105; A61K 31/505; A61K 38/1709; A61K 31/739; A61K 31/4439
USPC 514/16.4; 514/406; 514/44 A; 514/274; 514/54; 514/342

(57) ABSTRACT

Cardiac dysfunction during sepsis is due, at least in part, to cardiac energy deficiency. It has been discovered that lipopolysaccharide (LPS)-mediated cardiac dysfunction is prevented or treated by treatments that improve FA oxidation (FAO), despite the persistence of inflammation. The present invention relates to methods for increasing or maintaining cardiac function in a subject, by administering to the subject a therapeutically effective amount of an agent that increases fatty acid oxidation in the heart.

### CROSS-REFERENCE TO RELATED APPLICATIONS

This application claims benefit of U.S. Provisional Patent Application Ser. No. 61/445,002, entitled “Methods for Treating and Preventing Cardiac Dysfunction in Septic Shock,” filed on Feb. 21, 2011, the entire contents of which are hereby incorporated by reference as if fully set forth herein, under 35 U.S.C. §119 (e).

### STATEMENT OF GOVERNMENTAL INTEREST

This invention was made with Government support under Contract Nos. HL45095, HL73029, and T32 HL007343 awarded by National Heart, Lung, and Blood Institute of NIH. The Government has certain rights in the invention.

### BACKGROUND OF THE INVENTION

Sepsis is a major cause of death in intensive care units and is associated with cardiac dysfunction. Septic shock is characterized by hypotension, ischemia, and multiple organ failure and can lead to increased mortality. Cardiac dysfunction is a definitive consequence of severe sepsis and is characterized by impaired contractility, diastolic dysfunction, as well as reduced cardiac index and ejection fraction (EF). The mechanisms that underlie myocardial depression during septic shock are not well known. However, in an effort to further understand the complexity of cardiac dysfunction, the effects of sepsis have been reproduced in experimental models. The most common models used include injection of lipopolysaccharide (LPS), which is a bacterial cell wall component that induces pathophysiological consequences similar to those found during septic shock.

Cardiac septic shock is a severe condition, which besides its inflammatory component is characterized by dramatic reduction in metabolic rate. Given the high mortality associated with sepsis and the inability of anti-inflammatory treatment to improve mortality, other means of treating cardiac dysfunction associated with septic shock are needed. Therefore, there is a great need for new methods and compositions to treat and prevent cardiac dysfunction associated with septic shock.

### SUMMARY OF THE INVENTION

A first set of embodiments of the invention is directed to a method for increasing or maintaining cardiac function in a subject in need of such treatment, by administering to the subject a therapeutically effective amount of an agent that increases fatty acid oxidation (hereinafter “FAO”) in the heart. In certain embodiments, the subject has sepsis or is at risk of developing sepsis, or the subject has heart failure, or is at risk of developing heart failure.

In certain embodiments of the above method for increasing or maintaining cardiac function, the agent that increases fatty acid oxidation in the heart is a PPAR agonist selected from the group comprising PPARα agonists, PPARγ agonists, dual PPARα and PPAR γ agonists, or combinations thereof. The PPARα agonist is selected from the group comprising Alpha WY-14643, GW9578, GW-590735, K-111, LY-674, KRP-101, DRF-10945, LY518674, Propanoic Acid 2-[4-[3-[2,5-dihydro-1-[(4-methylphenyl)methyl]-5-oxo-1H-1,2,4-triazol-3-yl]propyl]phenoxy]-2-methyl, fibrate, fenofibrate, clofibrate, and bezafibrate. The PPARγ agonist may be selected from the group comprising hiazolidinedione, rosiglitazone, pioglitazone, MCC-555, GL-262570, englitazone, darglitazone, isaglitazone, JTT-501, T-895645, R-119702, N,N-2344, YM-440, thiazolidinedione, GI 262570, R-483 and rivoglitazone.

In certain other embodiments of the above method for increasing or maintaining cardiac function, the agent is an inhibitor of either c-Jun N-terminal kinase1 or 2 (JNK1 or JNK2 inhibitors) that increases fatty acid oxidation in the heart, including the JNK inhibitor SP600125. In another embodiment FAP is increased by reducing translation of mRNA encoding either JNK1 or JNK2, such as by administering therapeutically effective amounts of an antisense nucleic acid, siRNA, shRNA, micro RNA (miRNA), ribozyme, microRNA mimic, supermir, and aptamer that specifically hybridizes to JNK thereby reducing its expression.

Other embodiments include methods for increasing or maintaining cardiac function in a subject by administering a therapeutically effective amount of an agent that increases fatty acid oxidation in the heart such as aPPARα-coactivator-1 (PGC-1), an estrogen-related receptor (ERR)α, or a combination thereof.

Other embodiments are directed to various pharmaceutical compositions and kits including formulations comprising therapeutically effective amounts of agents that increase fatty acid oxidation in the heart and are effective in increasing or maintaining cardiac function in a subject. The formulations may comprise PPARα agonists, PPARγ agonists, dual PPARα and PPAR γ agonists, or combinations thereof, JNK inhibitors, or antisense nucleic acids, siRNAs, shRNAs, microRNAs (miRNA), ribozymes, microRNA mimics, supermirs, and aptamers, PPARα-co-activator-1 (PGC-1), estrogen-related receptor (ERR)α, or a combination thereof. Administration can be orally or intravenously. In certain embodiments the compositions are formulated for slow release to minimize the need for repeated delivery and to maintain a steady concentration of drugs.

Another set of embodiments is directed to methods for treating or preventing cardiac dysfunction in a subject having sepsis or at risk of developing sepsis, by administering therapeutically effective amounts of an agent that increases fatty acid oxidation in the heart. The above embodiments of the methods include administering the agent before cardiac function is diminished. The embodiments also comprise methods wherein the subject is treated with at least two members selected from the group comprising JNK inhibitors, PPARα agonists, PPARγ agonists, and dual PPARα and PPARγ agonists. In certain embodiments the two agents comprise a JNK inhibitor and a PPAR agonist. In certain embodiments the PPARγ agonist is either rosiglitazone administered in amounts of from about 0.1 to about 20 mg/day, and pioglitazone, administered in amounts of from about 0.1 to about 45 mg/day.

These and other features, aspects, and advantages of the present invention will become better understood with regard to the following description, appended claims, and accompanying drawings.

### BRIEF DESCRIPTION OF THE DRAWINGS

The present invention is illustrated by way of example, and not by way of limitation, the Figures.

FIG. 1: Sepsis is associated with increased inflammation but anti-inflammatory treatments do not prevent septic shock. Knockout animal models that are resistant to cardiac septic shock and anti-inflammatory therapies that were applied to humans failed to prevent septic shock.

FIG. 2: Sepsis is associated with reduced fatty acid oxidation. Effects of LPS on the expression of fatty acid metabolism-associated genes.

FIG. 3: Effects of LPS on (A) cardiac function, (B) plasma interleukin 6, and (C)-(D) gene expression levels of cardiac inflammatory cytokines and proteins that are associated with fatty acid metabolism.LPS induced inflammation and reduced cardiac function and expression of lipid metabolism-related genes in C57BL/6 mice.

FIG. 4: (A) Modulation of PPARα fatty acid and glucose utilization during the progression of heart failure. (B) Effects of LPS in the expression of cardiac genes that are associated with glucose metabolism. Heart failure reduces FAO and increases glucose utilization.

FIG. 5: (A)-(B) Several miRs (microRNAs) have been associated with heart failure related cardiac dysfunction in C57BL/6 mice. (C) Cardiac microRNA changes that occur in heart failure are not observed in sepsis-mediated heart dysfunction in C47BL/6 mice.

FIG. 6: Effects of LPS in gene expression levels of α- and β-myosin heavy chain (MHC) isoforms in the hearts of C57BL/6 mice. LPS reduces both αMHC and βMHC.

FIG. 7: Effects of LPS in the phosphorylation/activation of JNK and c-Jun in hearts of C57BL/6 mice. JNK and c-Jun are activated in LPS-treated C57BL/6 mice.

FIG. 8: (A)-(B) Protein levels of JNK2 (A) and p-cJun (B) in AC16 cells that were treated with adenovirus-expressing constitutively active JNK. (C) Effects of adenovirus-mediated expression of a constitutively active JNK in PPARα gene expression levels in a human cardiomyocyte cell line (AC16). (D) P-JNK levels in LPS-treated AC16 cells. (E) LPS-treatmentreduces PPARα expression in a human cardiomyocyte cell line. The effect of LPS on PPARα gene expression is prevented by combined treatment of cells with LPS and JNK inhibitor (SP600125).

FIG. 9: Effects of LPS in cardiac function and in PPARα gene expression levels of JNK2−/− and C57BL/6 mice. PPARα gene expression is reduced and JNK pathway is activated. (A) LPS does not affect cardiac function which is already reduced in JNK2−/− mice. (B) LPS dramatically reduces PPARα gene expression in JNK2−/− mice and in normal mice. (C) Effects of LPS in the phosphorylation/activation of JNK and c-Jun in hearts of JNK2−/− mice.

FIG. 10: Outline of the protocol for the treatment of C57BL/6 mice with JNK inhibitor (SP600125) and LPS.

FIG. 11: Effects of JNK inhibitor in the phosphorylation/activation of (A) c-Jun, (B) PPARα gene expression levels and (C) cardiac function. JNK inhibitor blocked the effects of LPS on PPARα gene expression and cardiac function in C57BL/6 mice.

FIG. 12: Effects of JNK inhibitor in (A) cardiac fatty acid oxidation, (B) PGC1-α and (C) Cpt1 gene expression levels. Cardiac function improvement by JNK inhibitor is associated with increased FAO.

FIG. 13: Effects of LPS and combined LPS and JNK inhibitor treatments in the expression of inflammatory cytokines genes (A) TNFα; (B) IL-6; and (C) IL-1α. Inflammation was not reduced in LPS-treated C57BL/6 mice that were injected with JNK inhibitor.

FIG. 14: Schematic representation of the proposed pathway for the beneficial effect of JNK inhibition in the prevention of LPS-mediated PPARα gene downregulation and in the improvement of fatty acid oxidation and cardiac function. JNK inhibitor increases PPARα which in turn normalizes cardiac function in LPS-treated C57BL/6 mice.

FIG. 15: Increased (A) fatty acid oxidation and (B) metabolic rate in mice with constitutive cardiomyocyte-specific PPARγexpression in PPARαdeficiency background. FAO is increased despite reduction of PPARα.

FIG. 16: Effects of LPS in (A) PPARα gene expression and in (B) cardiac function of αMHC-PPARγmice. Constitutive expression of PPARγ protects from LPS-mediated cardiac dysfunction despite PPARα downregulation.

FIG. 17: Effects of LPS in cardiac expression of inflammatory cytokines genes (A) IL-1α; (B) IL-6; and (C) TNFα of αMHC-PPARγ mice. Improved heart function in LPS-treated αMHC-PPARγmice is not associated with reduced inflammation.

FIG. 18: Protocol for the treatment of C57BL/6 mice with rosiglitazone and LPS.

FIG. 19: Effects of rosiglitazone in cardiac (A) PPARα and (B) PGC1α gene expression, in cardiac (C) fatty acid oxidation and in (D) cardiac function of LPS-treated C57BL/6 mice. Rosiglitazone increases FAO and protects from LPS-mediated cardiac dysfunction in normal mice.

FIG. 20: Effects of rosiglitazone in cardiac expression of inflammatory cytokines genes (A) TNF-α and (B) IL-1α of LPS-treated C57BL/6 mice. Inflammation was not affected by rosiglitazone treatment.

FIG. 21: Schematic representation of the role of rosiglitazone as a therapeutic in order to activate PPARγ and prevent LPS-mediated cardiac dysfunction by increasing fatty acid oxidation.

FIG. 22: Fractional shortening (A) and cardiac ATP content (B) of LPS-treated C57BL/6 and αMHC-PPARγ mice. (C) Fractional shortening of C57BL/6 mice treated with LPS and rosiglitazone. (D) Palmitate oxidation rate in hearts of LPS-treated αMHC-PPARγ mice and of C57BL/6 mice treated with LPS and rosiglitazone or JNKinh.

FIG. 23: LPS inhibits cardiac fatty acid oxidation and impairs cardiac function—(A) PPARα mRNA levels in hearts of 10- to 12-week-old C57BL/6 mice that were treated with 5 mg/kg LPS. n=5; **, p<0.01. (B, C) Photographs of echocardiograms (B) and fractional shortening (C) of 10- to 12-week-old C57BL/6 mice that were treated with 5 mg/kg LPS n=5; **, p<0.01. (D)[3H]palmitic acid oxidation in cardiac muscle of C57BL/6 mice treated with 5 mg/kg LPS. n=4; *, p<0.05. (E) ATP levels in hearts obtained from C57BL/6 mice that were treated with 5 mg/kg LPS; n=4; *, p<0.05. (F) Western blot analysis of pAMPK and total AMPK obtained from hearts of 10- to 12-week-old C57BL/6 mice that were treated with 5 mg/kg LPS. (G) Mitochondrial structure analysis and intracellular arrangement by electron microscopy of cardiac tissue from LPS-treated C57BL/6 mice showed that LPS treatment did not affect either the morphology or the intracellular arrangement of mitochondria as compared to control mice that were treated with saline.

FIG. 24: Constitutive expression of PPARγ in cardiomyocytes stimulates cardiac fatty acid oxidation and prevents LPS-mediated heart dysfunction despite elevated inflammation. (A) Western blot analysis of pAMPK, total AMPK, pJNK and total JNK obtained from hearts of 10- to 12-week-old αMHC-PPARγ mice that were treated with 5 mg/kg LPS. (B) The left bar represents the control and the right bar reflects LPS. ERRα, perilipin 2, perilipin 5, PGC-1α, PGC-1β, AOX, PDK4, PPARα, PPARδ, Cpt-1 and CD36 mRNA levels in hearts of 10- to 12-week-old αMHC-PPARγ mice that were treated with 5 mg/kg LPS. n=5; *, p<0.05, **, p<0.01. (C)[3H]palmitic acid oxidation in cardiac muscle of αMHC-PPARγ mice treated with 5 mg/kg LPS. n=4; *, p<0.05. (D) ATP levels in hearts obtained from αMHC-PPARγ mice that were treated with 5 mg/kg LPS; n=4; *, p<0.05. (E, F) Photographs of echocardiograms (E) and fractional shortening (F) of 10 weeks old αMHC-PPARγ mice that were treated with 5 mg/kg LPS n=4. (G-I) IL-1α (G), IL-6 (H) and TNFα (I) mRNA levels in hearts of 10 weeks old αMHC-PPARγ mice that were treated with 5 mg/kg LPS. n=5; **, p<0.01.

FIG. 25: Rosiglitazone-mediated activation of PPARγ prevents LPS-induced reduction in cardiac fatty acid oxidation and improves heart function despite elevated inflammation—(A, B) Photographs of echocardiograms (A) and fractional shortening (B) of 10-12 weeks old C57BL/6 mice that were treated with 5 mg/kg LPS or a combination of LPS and 35 mg/kg rosiglitazone (rosi). Control cells were treated with saline; n=4; *, p<0.05. (C) Western blot analysis of pAMPK, total AMPK, pJNK and total JNK obtained from hearts of C57BL/6 mice that were treated with 5 mg/kg LPS or a combination of LPS and 35 mg/kg rosiglitazone (Rosi). Control cells were treated with saline. (D)[3H]palmitic acid oxidation in cardiac muscle of 10-12 weeks old C57BL/6 mice that were treated with 5 mg/kg LPS or a combination of LPS and 35 mg/kg rosiglitazone (Rosi). Control cells were treated with saline; n=4; *, p<0.05. (E) Bar 1 of 4 depicts the control. Bar 2 of 4 depicts Rosi. Bar 3 of 4 depicts LPS. Bar 4 of 4 depicts a combination of Rosi and LPS. PPARα, PPARγ, PPARδ, ERRα, AOX, PGC-1α, PGC-1β, CD36, perilipin 2, perilipin 5 and PDK4 mRNA levels in hearts of C57BL/6 mice that were treated with 5 mg/kg LPS or a combination of LPS and 35 mg/kg rosiglitazone (Rosi). Control cells were treated with saline; n=5; *, p<0.05, **, p<0.01, +, p<0.001. (F-H) TNFα (F), IL-1α (G) and IL-6 (H) mRNA levels in hearts of C57BL/6 mice that were treated with 5 mg/kg LPS or a combination of LPS and 35 mg/kg rosiglitazone (Rosi). Control cells were treated with saline; n=5; *, p<0.05; **; p<0.01, +; p<0.001.

FIG. 26: Rosiglitazone improves survival of LPS-treated C57BL/6 mice—Survival curves of C57BL/6 mice that were treated with saline (n=10), 35 mg/kg/day rosiglitazone (n=10), 5 mg/kg LPS (n=14) or combination of rosiglitazone and LPS (n=12).

FIG. 27: Proposed model—(A) Schematic model that explains the role of the activation of PPARγ in the induction of fatty acid oxidation and prevention of LPS-mediated cardiac dysfunction. Binding of the complex that consists of LPS and lipopolysaccharide binding protein (LBP) on the TLR4 and CD14 receptors leads to downregulation of PPARα, which eventually causes inhibition of fatty acid oxidation and cardiac dysfunction. In addition, inflammatory pathways are activated via stimulation of the NF-κB signaling pathway. (B) Activation of PPARγ prevents sepsis-mediated down-regulation of cardiac fatty acid oxidation, thus it protects cardiac function in sepsis despite increased levels of inflammatory cytokines.

FIG. 28: Administration of PPARα agonist WY-14643 improved fractional shortening by 25%.

In the Summary of the Invention above and in the Detailed Description of the Invention, and the claims below, an in the accompanying drawings, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally. For the purposes of explanation, numerous specific details are set forth in order to provide a thorough understanding of the present invention. It will be apparent, however, to one skilled in the art that the present invention may be practiced without these specific details.

### DETAILED DESCRIPTION

It has now been discovered that lipopolysaccharide (LPS)-mediated cardiac dysfunction can be treated by administering agents that improve FAO, despite the persistence of inflammation, thereby providing a focused therapy for those in heart failure. These results have strong therapeutic implications for treating diseases or disorders associated with cardiac dysfunction during sepsis.

### DEFINITIONS

Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane Antibodies: A Laboratory Manual Cold Spring Harbor Laboratory Press, Cold Spring Harbor, N.Y. (1990); Principles of Neural Science, 4^{th} ed., Eric R. Kandel, James H. Schwart, Thomas M. Jessell editors. McGraw-Hill/Appleton & Lange: New York, N.Y. (2000). Unless defined otherwise, all technical and scientific terms used herein have the same meaning as commonly understood by one of ordinary skill in the art.

The terms “individual,” “subject,” and “patient” are used interchangeably herein and refer to any mammalian subject for whom diagnosis, treatment, or therapy is desired, particularly humans. A “subject” as used herein generally refers to any living multicellular organism. Subjects include, but are not limited to animals (e.g., cows, pigs, horses, sheep, dogs and cats) and plants, including hominoids (e.g., humans, chimpanzees, and monkeys). The term includes transgenic and cloned species. The term “patient” refers to both human and veterinary subjects.

“Administering” shall mean delivering in a manner which is affected or performed using any of the various methods and delivery systems known to those skilled in the art. Administering can be performed, for example, orally, or intravenously, via implant, transmucosally, transdermally, intradermally, intramuscularly, subcutaneously, or intraperitoneally. Administering can also be performed, for example, once, a plurality of times, and/or over one or more extended periods.

The phrase “therapeutically effective amount” means an amount sufficient to produce a therapeutic result. Generally the therapeutic result is an objective or subjective improvement of a disease or condition, achieved by inducing or enhancing a physiological process, blocking or inhibiting a physiological process, or in general terms performing a biological function that helps in or contributes to the elimination or abatement of the disease or condition. For example, eliminating or reducing or mitigating the severity of a disease or set of one or more symptoms. The full therapeutic effect does not necessarily occur by administration of one dose and may occur only after administration of a series of doses. Thus, a therapeutically effective amount may be administered in one or more administrations. A therapeutic effective amount further includes an amount to maintain cardiac function in a subject with sepsis or is at risk of having sepsis.

A subject is “at a high risk of sepsis” where a subject has a condition that reduces the ability to fight serious infections. These conditions include having a weakened immune system—due to use of drugs that suppress the immune system (such as chemotherapy drugs or corticosteroids) or due to certain disorders (such as cancer, AIDS, and immune disorders, spinal cord injuries and certain blood disorders. The risk is also increased in people who are more likely to have bacteria enter their bloodstream. Such people include those who have a medical device inserted into the body (such as a catheter inserted into a vein or the urinary tract, drainage tubes, or breathing tubes). When medical devices are inserted, they can move bacteria into the body. Bacteria may also collect on the surface of such devices, making infection and sepsis more likely. The longer the device is left in place, the greater the risk. Other conditions also increase the risk of sepsis: (i) injecting recreational drugs where the drugs and needles used are rarely sterile; (ii) having an artificial (prosthetic) joint or heart valve or certain heart valve abnormalities as bacteria tend to lodge and collect on these structures and can be released into the blood stream; and (iii) having an infection that persists despite treatment with antibiotics: as some bacteria that cause infections and sepsis are resistant to antibiotics and that can cause sepsis. A physician will be able to identify patients for whom intervention by the methods of the present invention is warranted.

Treating a disease means taking steps to obtain beneficial or desired results, including clinical results, such as mitigating, alleviating or ameliorating one or more symptoms of a disease; diminishing the extent of disease; delaying or slowing disease progression; ameliorating and palliating or stabilizing a metric (statistic) of disease. “Treatment” refers to the steps taken.

“Mitigating” means reducing or ameliorating a disease or symptom of a disease. For example, mitigation can be achieved by administering a therapeutic agent before the phenotypic expression of the disease (i.e. prior to the appearance of symptoms of the disease) Mitigation includes making the effects of disease less severe by avoiding, containing, reducing or removing it or a symptom of it. Mitigating an enumerated disease as described herein comes within the definition of “treating” an enumerated disease before symptoms occur. Amounts of therapeutic agents that mitigate a disease are herein referred to as “therapeutically effective amounts.”

“Significantly higher” or “significantly increased” is at least about a 15% increase over control levels or pretreatment levels whereas “significantly reduced” is at least about a 15% decrease over control levels or pretreatment levels.

By “maintaining cardiac function” is meant that the level of cardiac function remains essentially unchanged, i.e. further loss of cardiac function is prevented.

“Agent” means PPARα agonists, PPARγ agonists, dual PPARα and PPARγ agonists, or combinations thereof or JNK inhibitors such as SP600125. The agent may be an antisense nucleic acid, siRNA, shRNA, microRNA (miRNA), ribozyme, microRNA mimic, supermir, or aptamer that specifically hybridizes to JNK1 or 2 and known JNK1 and 2 inhibitors. An agent may be a PPARα-coactivator-1 (PGC-1), estrogen-related receptor (ERR)α or combination thereof. PPARα agents may be Alpha WY-14643, GW9578, GW-590735, K-111, LY-674, KRP-101, DRF-10945, LY518674, Propanoic Acid 2-[4-[3-[2,5-dihydro-1-[(4-methylphenyl)methyl]-5-oxo-1H-1,2,4-triazol-3-yl]propyl]phenoxy]-2-methyl, fibrate, fenofibrate, clofibrate, and bezafibrate.PPARγ agonists may be hiazolidinedione, rosiglitazone, pioglitazone, MCC-555, GL-262570, englitazone, darglitazone, isaglitazone, JTT-501, T-895645, R-119702, N,N-2344, YM-440, thiazolidinedione, GI 262570, R-483 and rivoglitazone. These “agents” increase cardiac function or maintain cardiac function in subjects with sepsis by increasing fatty acid oxidation in the heart.

“MiR” also “micro RNA” means a newly discovered class of small non-coding RNAs that are key negative regulators of gene expression Like conventional protein-encoding RNA, miRs are transcribed by RNA polymerase II and their expression is controlled by transcriptional factors. The mature miRs inhibit target mRNA translation or promote their degradation by directly binding to specific miR binding sites in the 3′-untranslated region (3′-UTR) of target genes.

“siRNA” means small interfering RNA, sometimes known as short interfering RNA or silencing RNA, and is a class of double-stranded RNA molecules, 20-25 nucleotides in length, that play a variety of roles in biology. The most notable role of siRNA is its involvement in the RNA interference (RNAi) pathway, where it interferes with the expression of a specific gene.

“shRNA” means a small hairpin RNA or short hairpin RNA, that is a sequence of RNA that makes a tight hairpin turn that can be used to silence gene expression via RNA interference. shRNA uses a vector introduced into cells and utilizes the U6 or H1 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it. shRNA is transcribed by RNA polymerase III. shRNA production in a mammalian cell can sometimes cause the cell to mount an interferon response as the cell seeks to defend itself from what it perceives as viral attack.

“Ribozyme” is an RNA molecule with a well-defined tertiary structure that enables it to catalyze a chemical reaction. Ribozyme means ribonucleic acid enzyme. It may also be called an RNAenzyme or catalytic RNA. It contains an active site that consists entirely of RNA. Many natural ribozymes catalyze either the cleavage of one of their own phosphodiester bonds (self-cleaving ribozymes), or the cleavage of bonds in other RNAs. Some have been found to catalyze the aminotransferase activity of the ribosome.

“Supermirs” refer to single stranded, double stranded or partially double stranded oligomers or polymers of RNA or DNA or both, which has a nucleotide sequence that is substantially identical to a miRNA and that is antisense with respect to its mRNA target.

“Aptamers” are oligonucleic acid or peptide molecules that bind to a specific target molecule. Aptamers are usually created by selecting them from a large random sequence pool, but natural aptamers also exist in riboswitches. Aptamers can be used for both basic research and clinical purposes as macromolecular drugs. Aptamers can be combined with ribozymes to self-cleave in the presence of their target molecule. These compound molecules have additional research, industrial and clinical applications.

“ERRα” or Estrogen-related receptor alpha, also known as NR3B 1 (nuclear receptor subfamily 3, group B, member 1), is a nuclear receptor that in humans is encoded by the ESRRA (Estrogen Related Receptor Alpha) gene.

“c-Jun N-terminal kinase (JNK) is a member of the mitogen-activated protein kinase (MAPK) family and plays an essential role in TLR-mediated inflammatory responses.

“Treating” a subject afflicted with a disorder shall mean causing the subject to experience a reduction, delayed progression, regression or remission of the disorder and/or its symptoms. In one embodiment, recurrence of the disorder and/or its symptoms is prevented. In the preferred embodiment, the subject is cured of the disorder and/or its symptoms.

### BACKGROUND

Cardiac dysfunction during sepsis is due, at least in part, to cardiac energy deficiency. It has now been discovered that lipopolysaccharide (LPS)-mediated cardiac dysfunction can be both treated (mitigated and prevented) by administering agents that improve FAO. These agents include PPAR agonists and JNK inhibitors. As will be described in detail, the addition of JNK inhibitors and PPAR agonists administered within 24 hours of LPS prevented the typical LPS-induced increase in FAO and as a result, also prevented cardiac dysfunction. Thus, increasing FAO preferably within 24 hours of diagnosing sepsis, upregulated PPARα expression, maintained or increased FAO, and rescued cardiac function despite elevated expression of cardiac inflammatory markers. Without being bound by theory, PPARγ activation may compensate for LPS-mediated reduction of PPARα thereby restoring FAO and cardiac function. Because the agents were able to prevent the expected loss of cardiac function even after sepsis had begun and even though inflammation persisted, certain claims are directed to a method for mitigating and preventing cardiac dysfunction as well as treating it in sepsis by increasing FAO.

### EMBODIMENTS OF THE INVENTION

Cardiac dysfunction is a definitive consequence of severe sepsis and is characterized by impaired contractility, diastolic dysfunction, as well as reduced cardiac index and ejection fraction (EF). In an effort to investigate cardiac dysfunction, the effects of septic shock have been reproduced in experimental models of sepsis. The most common models include injection of lipopolysaccharide (LPS). LPS is a bacterial cell wall component that induces pathophysiological consequences similar to those found during septic shock. With the use of this LPS model, it has now been discovered that LPS-mediated cardiac dysfunction can be both treated and prevented by administration of with agents that improve fatty acid oxidation in the heart. Optimal results can be achieved if the therapeutic agents (JNK inhibitors and PPAR agonists) are administered within 24 hours after a subject is diagnosed as having sepsis or is at risk of developing sepsis.

As is described in the Summary of the Invention, some embodiments are directed to methods for increasing or maintaining cardiac function in a subject heart in a subject in need of such treatment, particularly by increasing fatty acid oxidation in the heart, for example in a subject that has sepsis or is at risk of developing sepsis, or has heart failure, or is at risk of developing heart failure. By increasing or maintaining cardiac function is meant that the post-treatment level of cardiac function is “significantly higher” or “significantly increased” to at least about a 15% increase over control levels or pretreatment levels.

The desired increase in fatty acid oxidation in the heart is a result of administering therapeutically effective amounts of agents as described herein, including PPAR agonists, JNK inhibitors (e.g., SP600125), or antisense nucleic acids, siRNAs, micro RNAs (miRNA), short hairpin RNAs (shRNA), ribozymes, microRNA mimics, supermirs, and aptamers that specifically hybridize to JNK1 or JNK2, thereby reducing its expression and PPARα-coactivators-1, estrogen-related receptors, or combinations thereof.

PPARα agonists that increase fatty acid oxidation include those in the group comprising Alpha WY-14643, GW9578, GW-590735, K-111, LY-674, KRP-101, DRF-10945, LY518674, Propanoic Acid 2-[4-[3-[2,5-dihydro-1-[(4-methylphenyl)methyl]-5-oxo-1H-1,2,4-triazol-3-yl]propyl]phenoxy]-2-methyl, fibrate, fenofibrate, clofibrate, and bezafibrate.

PPARγ agonists increase FAO in type 2 diabetic human muscle cells. PPARγ agonists that will prevent or alleviate sepsis-mediated heart dysfunction and mortality, include those selected from the group comprising hiazolidinedione, rosiglitazone, pioglitazone, MCC-555, GL-262570, englitazone, darglitazone, isaglitazone, JTT-501, T-895645, R-119702, N,N-2344, YM-440, thiazolidinedione, GI 262570, R-483, and rivoglitazone.

Other embodiments are directed to methods for treating or preventing cardiac dysfunction in a subject having sepsis or at risk of developing sepsis, by administering therapeutically effective amounts of an agent that increases fatty acid oxidation in the heart. The above embodiments of the methods include administering the agent before cardiac function is diminished. The embodiments also comprise methods wherein the agent comprises at least two members selected from the group comprising JNK inhibitors, PPARα agonists, PPARγ agonists, and dual PPARα and PPARγ agonists. In certain embodiments the two agents comprise a JNK inhibitor and a PPAR agonist. Embodiments can also comprise methods for treating or preventing cardiac dysfunction in a subject having heart failure, by administering therapeutically effective amounts of an agent that increases fatty acid oxidation in the heart.

Certain embodiments of the invention provide pharmaceutical compositions and kits including the agents that increase fatty acid oxidation described herein. In preferred embodiments, the pharmaceutical formulations comprise therapeutically effective amounts of at least two of the following groups: (i) JNK1 or a JNK2 inhibitors including antisense nucleic acids, siRNAs, shRNAs, microRNAs (miRNA), ribozymes, microRNA mimics, supermirs, and aptamers; (ii) PPAR agonists selected from the group comprising PPAR agonists, PPARγ agonists, dual PPARα and PPAR γ agonists; and (iii) PPARα-co-activator-1 (PGC-1) and (iii) estrogen-related receptor (ERR), which amounts treat or prevent cardiac dysfunction in a subject having heart failure or sepsisor at risk of developing them; or in amounts that increase cardiac function also in the patient having heart failure or sepsis or at risk of developing them. It has been reported that MiR-520b decreases the levels of p-JNK and siMEKK2 abolishes levels of p-JNK in hepatoma cells.²¹⁶

The amount of PPAR agonist and JNK inhibitors to administer will vary as described below. The 2005 Physician's Desk Reference (PDR) describes administering an oral formulation of rosiglitazone in amounts of from about 8 mg/day to about 20 mg/day for treating diabetes (page 1442), and pioglitazone in amounts of from about 8 mg/day to about 45 mg/day (Page 3185). Pioglitazone is preferred as it has fewer deleterious effects on blood lipids.

In the experiments described herein, an injectable formulation of rosiglitazone was made that was administered intraperitoneally in amounts of 35 mg/kg, however lower doses of 8.5 mg/kg-16.5 mg/kg were also effective. Data not shown. The efficacy of the intraperitoneally injectable form of rosiglitazone shows that it need not be administered orally to be effective. Routine experimentation will determine the optimal dose of rosiglitazone and pioglitazone, and the optimal formulation and route of administration. In certain embodiments this dose range is from about 0.1 mg/day to about 45 mg/day. Determination of the optimal doses of other PPAR agonists and JNK inhibitors can be accomplished by beginning with administering FDA approved doses and formulations for each respective agent that has been FDA-approved, or for FDA-approved agents that are similar. For example, the following agents are FDA approved: (i) Fibrates including Fenofibrate (43 mg daily FDA approved), Clofibrate (500 mg daily FDA approved), and Bezafibrate (400 mg daily FDA approved) and Gemfibrozil (600 mg daily); and (ii) Thiazolidinedione which is a generic term that includes rosiglitazone and, pioglitazone (15 mg, FDA approved).

Treatment of acute cardiac function associated with sepsis is short term, therefore drugs that have long term toxic side effects can be used at the physician's discretion. The progress of this therapy is easily monitored by conventional techniques and assays that may be used to adjust dosage to achieve a desired therapeutic effect.

To summarize, cardiac function can be increased in a subject, and sepsis-mediated heart dysfunction can be prevented or treated by stimulation of FA oxidation in cardiomyocytes: (i) via inhibition of JNK by JNK inhibitors (e.g., SP600125), or antisense nucleic acids, siRNAs, micro RNAs (miRNA), short hairpin RNAs (shRNA), ribozymes, microRNA mimics, supermirs, and aptamers, that specifically hybridize to JNK thereby reducing its expression; (ii) via activation of PPARγ using agonists such as rosiglitazone and dual PPARα/PPARγ agonists; (iii) via activation of PPARα using agonists (alpha agonists and dual PPARα/PPARγ); (iv) via activation of both PPARα/PPARγ with agonists; and (v) via PPAR α-coactivator-1 (PGC-1) and/or estrogen related receptor (ERR)α, to activate PPARα. The agents are preferably administered as soon as possible after a subject is identified as being at risk of developing sepsis or as having sepsis, preferably before any cardiac function has been lost or diminished.

### Overview

It is well established that nuclear factor kappa-light-chain-enhancer of activated B cells (NF-κB)¹⁴ and c-Jun N-terminal kinases (JNK)^{15,16} are targets of LPS stimulus, and they are both inducers of production of inflammatory response-related cytokines, such as TNFα and interleukin IL-1 and IL-8. However, anti-inflammatory driven therapies, such as administration of corticosteroids,¹⁷⁻¹⁹ IL-1 receptor antagonists,^{20,21} or anti-TNFα²² have not improved patients' mortality. Therefore, besides inflammation, sepsis-mediated effects must also involve other pathophysiologic mechanisms.

Studies involving LPS administration have resulted in the conclusion that LPS administration leads to profound changes in cardiac energy balance with a dramatic reduction in fatty acid oxidation.²³⁻²⁵ Hearts rely mostly on fatty acid catabolism for their energy needs.³² In fact, approximately 70% of the produced energy results from fatty acid oxidation. In pathologic conditions that are characterized by reduced cardiac work such as heart failure, it has been documented that fatty acid oxidation is reduced^{33,34} and glucose oxidation is increased. In contrast, LPS-mediated reduction in cardiac fatty acid oxidation is not compensated by increased glucose catabolism.³⁵ Thus, sepsis is accompanied by cardiac energy production deficiency.

Fatty acid oxidation involves peroxisomes and mitochondria, and it is regulated by several enzymes and transcription factors. Nuclear receptors, particularly PPARs, have a major role in the control of fatty acid oxidation. Different PPARs have different tissue distribution and modulate different physiological functions. The PPARs play a key role in various aspects of the regulation of a large number of genes, the products of which genes are directly or indirectly crucially involved in lipid and carbohydrate metabolism.

PPARs have attracted considerable scientific attention in the last few years in part because of their emergence as the molecular target of a new group of Type II (non-insulin dependent) diabetes (NIDDM) medicines, the glitazones. PPARs are nuclear transcription factors that can be activated by ligands and belong to the class of nuclear hormone receptors. Three PPAR isoforms exist: (i) PPARα, (ii) PPARγ, and (iii) PPARδ (identical to PPARβ), and are encoded by different genes.¹⁹⁶In humans, PPARγ exists in three variants, PPARγ₁, PPARγ₂, and PPARγ₃, which are the result of alternative use of promoters and differential mRNA splicing.

A marked reduction of cardiac PPARα accompanies LPS administration.^{28,43}Recent studies²⁰⁰ showed that JNK inhibits PPARα gene expression. PPARα-mediated fatty acid oxidation in the heart and other tissues relies on the activation of peroxisomal and mitochondrial enzymes such as, acyl-CoA oxidase (AOX) and carnitine palmitoyl-transferase I (CptI). A major co-activator of PPARα-mediated fatty acid oxidation, at the transcriptional level, is the PPARγ-coactivator-1 (PGC-1).⁴⁴Many different factors that are known to increase PPARα expression, include glucocorticoids⁴⁵, farnesoid X receptor (FXR)⁴⁶, AMP-activated protein kinase (AMPK)⁴⁷⁻⁴⁹, estrogen related receptor (ERR)α⁵⁰, retinoic acid⁵¹, retinoid X receptor (R×R)⁵², phorbol-12-myristate-13-acetate⁵³, exercise training⁵⁴, heat shock factor-1⁵⁵ and others.

PPARα expression is downregulated by other factors including heart failure⁵⁶, myocardial infarction,⁵⁷ hypoxia,^{58,59} IL-1β,⁶⁰ IL-6,⁶⁰ PPAR,δ^{61,62} NF-κB,⁶³ glucose,^{64,65} insulin,⁶⁶ Akt,⁶⁷ c-Myc,⁶⁸ the Janus kinase/signal transducers and activators of transcription (JAK/STAT) pathway,⁶⁹ reactive oxygen species,⁶³ growth hormone,⁷⁰ androgens,⁷¹ and angiotensin II.⁷² PPARα gene expression levels and subsequent fatty acid oxidation are upregulated by estrogen related receptor (ERR)α, which acts in conjunction with PGC-1α and binds directly to the PPARα promoter.⁵⁰ In addition, ERRα gene expression is induced by PGC-1α^{50,73} at the transcriptional level indicating a positive feedback loop in the coordination of PGC-1a and ERRα towards increase of the PPARα gene expression.

PPARγ, besides its well-known role as a major regulator of lipogenesis,^{39,40} also contributes to fatty acid oxidation in cardiac⁴¹ and skeletal⁴² muscle. Mice with constitutive PPARγ expression specifically in the hearts of PPARα−/− mice (αMHC-PPARγ/PPARα^{-/-}) have elevated fatty acid oxidation levels, as well as improved cardiac function and survival as compared to the cardiolipotoxic αMHC-PPARγ mice.⁴¹ These observations indicated that an overexpression of PPARγ can substitute for PPARα. PPARγ agonists have also been shown to increase fatty acid oxidation in type 2 diabetic human muscle cells.⁴² Thus, one method to overcome a marked reduction in PPARα might be via overexpression of other members of the PPAR gene family.

In both PPARα-dependent and -independent mechanisms, PGC-1 is an important component for fatty acid oxidation. PGC-1 is a coactivator of several cellular energy metabolism-related transcription factors such as PPARs, estrogen related receptors (ERR), LXR, thyroid hormone receptor, retinoid receptors, glucocorticoid receptor, estrogen receptor and others.¹²¹PGC-1α expression is downregulated by LPS and the JNK signaling pathway.¹²²

### SUMMARY OF RESULTS

The results herein show that certain agents that increase fatty acid oxidation in the heart affected by sepsis ultimately increase or maintain cardiac function with the above-described therapeutic implications. Sepsis-mediated heart dysfunction can be prevented and treated by stimulation of FAO in cardiomyocytes: (i) via inhibition of JNK by JNK inhibitors (e.g., SP600125), (Examples 4 and 9) or antisense nucleic acids, siRNAs, micro RNAs (miRNA), short hairpin RNAs (shRNA), ribozymes, microRNA mimics, supermirs, and aptamers, that specifically hybridize to JNK thereby reducing its expression; (ii) via activation of PPARγ using agonists such as rosiglitazone and dual PPARα/PPARγ agonists; (Examples 5 and 10-12) (iii) via activation of PPARα using agonists (alpha agonists and dual PPARα/PPARγ) (Example 8); (iv) via activation of both PPARα/PPARγ with agonists; and (v) via PPAR α-coactivator-1 (PGC-1) and/or estrogen related receptor (ERR)α, to activate PPARα. Details are set forth in the Examples.

### Therapeutic Oligonucleotides

Based on these known sequences of the targeted miRs or mRNA and the genes encoding them, therapeutic oligonucleotides can be engineered using methods known in the art. These oligonucleotides include antisense DNA or RNA (or chimeras thereof), small interfering RNA (siRNA), micro RNA (miRNA), short hairpin RNA, ribozymes, microRNA mimic, supermir, and aptamers. Different combinations of these therapeutic agents can be formulated for administration to a subject using methods well known in the art.

The siRNA Oblimersen (Genasense®) has been given to patients for up to six cycles of 7 days at a 3 mg/kg/day dose with no severe adverse effects. Oligonucleotides are relatively safe, and have been administered at doses of up to 15 mg/kg to non-human primates. Webb M S, et al. Antisense Nucleic Acid Drug Dev. 2001; 11:155; O'Brien S, et al. J. Clin. Oncol. 2007; 25: 1114. Therapeutic oligonucleotides have been administered in amounts ranging from about 0.1 mg/kg to about 50 mg/kg, and can be delivered for example, intravenously. MOLECULAR THERAPY Vol, 13, No. 4, April 2006 Administration of the therapeutic agents or compositions of this invention, may be accomplished using any of the conventionally accepted modes of administration, and doses vary based on the severity and type of disorder, and on the patient, doses may be on the lower or higher end of the spectrum.

The nucleic acid sequences of the human JNK suitable for targeting are in the public domain are:
Homo sapiens JNK1 NCBI Reference Sequence: NG_{—}029053.1.
Homo sapiens JNK2 NCBI Reference Sequence: NG_{—}029059.1
Homo sapiens JNK3NCBI Reference Sequence: NG_{—}013325.1.

Since JNK3 is expressed mostly in neural tissues, it is not considered a major target in the heart.

The mRNA and gene sequences encoding JNK are set forth by accession numbers.

For JNK1:

JNK1 GenBank: L26318.1 (Human protein kinase (JNK1) mRNA), mRNA: BC144063 (Homo sapiens mitogen-activated protein kinase 8, mRNA), Gene map locus: 10q11.2,
JNK1a1:NP_{—}002741(mitogen-activated protein kinase 8 isoform JNK1 alpha1 [Homo sapiens]),
JNK1a2: NP_{—}620637(mitogen-activated protein kinase 8 isoform JNK1 alpha2 [Homo sapiens]),

### JNK1β1: NP_{—}620634, JNK1β2: NP_{—}620635.

For JNK2:

JNK2 GenBank: U09759.1, mRNA: U09759.1, Gene map locus:5q35, JNK2α1: NP_{—}620707, JNK2α2: NP_{—}002743, JNK2β1: NP_{—}620708, JNK2β2: NP_{—}620709, JNK2γ: NP_{—}001128516. JNK3 GenBank: BC035057.1 (Homo sapiens mitogen-activated protein kinase 10, mRNA), mRNA:BCO22492 (Homo sapiens mitogen-activated protein kinase 10, mRNA), Gene map locus: 4q21.3.

For JNK3:

JNK3α1: AAC50605 (JNK3 alpha1 protein kinase [Homo sapiens]), and JNK3α2: AAC50604.

The nucleotide sequences of mouse c-Jun N-terminal kinase JNK1, -2, and -3 cDNAs have been deposited in DDBJ/EMBL/GenBank with accession nos. AB005663, AB005664, and AB005665, respectively. It has been reported that MiR-520b decreases the levels of p-JNK in human hepatoma cells.²¹⁶ Therefore the same MiR can be used to inhibit JNK in embodiments of the present invention.

The oligonucleotides that may be used as agents herein are synthesized in vitro and do not include compositions of biological origin. Based on these known sequences of the targeted miRs or mRNA and the genes encoding them, therapeutic oligonucleotides can be engineered using methods known in the art. These oligonucleotides include antisense DNA or RNA (or chimeras thereof), small interfering RNAs (siRNA), micro RNAs (miRNA), short hairpin RNAs, ribozymes, microRNA mimics, supermirs, and aptamers. Different combinations of these therapeutic agents can be formulated for administration to a subject using methods well known in the art. Certain embodiments of the present invention involve the therapeutic use of antisense nucleic acids or inhibitory RNAs such as small interfering RNA (siRNA) or short hairpin RNAs (shRNA) to reduce or inhibit expression and hence the biological activity of the certain targeted miRs or mRNA.

Therapeutic nucleic acids include, e.g., small interfering RNA (siRNA), shRNA, micro RNA (miRNA), antisense oligonucleotides, ribozymes, microRNA mimics, supermirs, and aptamers. These nucleic acids act via a variety of mechanisms. siRNA or miRNA can down-regulate intracellular levels of specific proteins through a process termed RNA interference (RNAi). Following introduction of siRNA or miRNA into the cell cytoplasm, these double-stranded RNA constructs can bind to a protein termed RISC. RNA-Induced Silencing Complex, or RISC, is a multiprotein complex that incorporates one strand of a small interfering RNA (siRNA) or micro RNA (miRNA). RISC uses the siRNA or miRNA as a template for recognizing complementary mRNA. When it finds a complementary strand, it activates RNase and cleaves the RNA. This process is important both in gene regulation by microRNAs and in defense against viral infections, which often use double-stranded RNA as an infectious vector.RNAi can provide down-regulation of specific proteins by targeting specific destruction of the corresponding mRNA that encodes for protein synthesis.

The therapeutic applications of RNAi are extremely broad, since siRNA and miRNA constructs can be synthesized with any nucleotide sequence directed against a gene or mRNA encoding a target protein. To date, siRNA constructs have shown the ability to specifically down-regulate target proteins in both in vitro and in vivo models and they are currently being evaluated in clinical studies.

Antisense oligonucleotides and ribozymes can also inhibit mRNA translation into protein. In the case of antisense constructs, these single stranded deoxynucleic acids have a complementary sequence to that of the target protein mRNA and can bind to the mRNA by Watson-Crick base pairing. This binding either prevents translation of the target mRNA and/or triggers RNase H degradation of the mRNA transcripts. Consequently, antisense oligonucleotides have tremendous potential for specificity of action (i.e., down-regulation of a specific disease-related protein). To date, these compounds have shown promise in several in vitro and in vivo models, including models of inflammatory disease, cancer, and HIV (reviewed in Agrawal, Trends in Biotech. 14:376-387 (1996)). Antisense can also affect cellular activity by hybridizing specifically with chromosomal DNA. Advanced human clinical assessments of several antisense drugs are currently underway.

It is desirable to optimize the stability of the phosphodiester internucleotide linkage and minimize its susceptibility to exonucleases and endonucleases in serum. (Zelphati, O., et al., Antisense. Res. Dev. 3:323-338 (1993); and Thierry, A. R., et al., pp 147-161 in Gene Regulation: Biology of Antisense RNA and DNA (Eds. Erickson, R P and Izant, J G; Raven Press, NY (1992)).

Therapeutic nucleic acids being currently being developed do not employ the basic phosphodiester chemistry found in natural nucleic acids, because of these and other known problems. Modifications have been made at the internucleotide phosphodiester bridge (e.g., using phosphorothioate, methylphosphonate or phosphoramidate linkages), at the nucleotide base (e.g., 5-propynyl-pyrimidines), or at the sugar (e.g., 2′-modified sugars) (Uhlmann E., et al. Antisense: Chemical Modifications. Encyclopedia of Cancer, Vol. X., pp 64-81 Academic Press Inc. (1997)). Others have attempted to improve stability using 2′-5′ sugar linkages (see, e.g., U.S. Pat. No. 5,532,130).

Small interfering RNA (siRNA) has essentially replaced antisense ODN and ribozymes as the next generation of targeted oligonucleotide drugs under development. SiRNAs are RNA duplexes normally 16-30 nucleotides long that can associate with a cytoplasmic multi-protein complex known as RNAi-induced silencing complex (RISC). RISC loaded with siRNA mediates the degradation of homologous mRNA transcripts; therefore siRNA can be designed to knock down protein expression with high specificity. Unlike other antisense technologies, siRNA function through a natural mechanism evolved to control gene expression through non-coding RNA. This is generally considered to be the reason why their activity is more potent in vitro and in vivo than either antisense ODN or ribozymes. A variety of RNAi reagents, including siRNAs targeting clinically relevant targets, are currently under pharmaceutical development, as described, e.g., in de Fougerolles, A. et al., Nature Reviews 6:443-453 (2007).

While the first described RNAi molecules were RNA:RNA hybrids comprising both an RNA sense and an RNA antisense strand, it has now been demonstrated that DNA sense:RNA antisense hybrids, RNA sense:DNA antisense hybrids, and DNA:DNA hybrids are capable of mediating RNAi (Lamberton, J. S, and Christian, A. T., (2003) Molecular Biotechnology 24:111-119). Thus, the invention includes the use of RNAi molecules comprising any of these different types of double-stranded molecules. In addition, it is understood that RNAi molecules may be used and introduced to cells in a variety of forms. Accordingly, as used herein, RNAi molecules encompasses any and all molecules capable of inducing an RNAi response in cells, including, but not limited to, double-stranded oligonucleotides comprising two separate strands, i.e. a sense strand and an antisense strand, e.g., small interfering RNA (siRNA); double-stranded oligonucleotide comprising two separate strands that are linked together by non-nucleotidyl linker; oligonucleotides comprising a hairpin loop of complementary sequences, which forms a double-stranded region, e.g., shRNAi molecules, and expression vectors that express one or more polynucleotides capable of forming a double-stranded polynucleotide alone or in combination with another polynucleotide.

A “single strand siRNA compound” as used herein, is a siRNA compound which is made up of a single molecule. It may include a duplexed region, formed by intra-strand pairing, e.g., it may be, or include, a hairpin or pan-handle structure. Single strand siRNA compounds may be antisense with regard to the target molecule.

A single strand siRNA compound may be sufficiently long that it can enter the RISC and participate in RISC mediated cleavage of a target mRNA. A single strand siRNA compound is typically at least 14, and in other embodiments at least 15, 20, 25, 29, 35, 40, or 50 nucleotides in length. In certain embodiments, it is less than 200, 100, or 60 nucleotides in length.

Hairpin siRNA compounds will have a duplex region equal to or at least 17, 18, 19, 29, 21, 22, 23, 24, or 25 nucleotide pairs. The duplex region will may be equal to or less than 200, 100, or 50, in length. In certain embodiments, ranges for the duplex region are 15-30, 17 to 23, 19 to 23, and 19 to 21 nucleotides pairs in length. The hairpin may have a single strand overhang or terminal unpaired region. In certain embodiments, the overhangs are 2-3 nucleotides in length. In some embodiments, the overhang is at the sense side of the hairpin and in some embodiments on the antisense side of the hairpin.

A “double stranded siRNA compound” as used herein, is a siRNA compound which includes more than one, and in some cases two, strands in which interchain hybridization can form a region of duplex structure.

The antisense strand of a double stranded siRNA compound may be equal to or at least, 14, 15, 16 17, 18, 19, 25, 29, 40, or 60 nucleotides in length. It may be equal to or less than 200, 100, or 50, nucleotides in length. Ranges may be 17 to 25, 19 to 23, and 19 to 21 nucleotides in length. As used herein, term “antisense strand” means the strand of a siRNA compound that is sufficiently complementary to a target molecule, e.g. a target RNA.

The sense strand of a double stranded siRNA compound may be equal to or at least 14, 15, 16 17, 18, 19, 25, 29, 40, or 60 nucleotides in length. It may be equal to or less than 200, 100, or 50, nucleotides in length. Ranges may be 17 to 25, 19 to 23, and 19 to 21 nucleotides in length.

The double strand portion of a double stranded siRNA compound may be equal to or at least, 14, 15, 16 17, 18, 19, 20, 21, 22, 23, 24, 25, 29, 40, or 60 nucleotide pairs in length. It may be equal to or less than 200, 100, or 50, nucleotides pairs in length. Ranges may be 15-30, 17 to 23, 19 to 23, and 19 to 21 nucleotides pairs in length.

In many embodiments, the siRNA compound is sufficiently large that it can be cleaved by an endogenous molecule, e.g., by Dicer, to produce smaller siRNA compounds, e.g., siRNAs agents.

The sense and antisense strands may be chosen such that the double-stranded siRNA compound includes a single strand or unpaired region at one or both ends of the molecule. Thus, a double-stranded siRNA compound may contain sense and antisense strands, paired to contain an overhang, e.g., one or two 5′ or 3′ overhangs, or a 3′ overhang of 1-3 nucleotides. The overhangs can be the result of one strand being longer than the other, or the result of two strands of the same length being staggered. Some embodiments will have at least one 3′ overhang. In one embodiment, both ends of a siRNA molecule will have a 3′ overhang. In some embodiments, the overhang is 2 nucleotides.

In certain embodiments, the length for the duplexed region is between 15 and 30, or 18, 19, 20, 21, 22, and 23 nucleotides in length, e.g., in the ssiRNA compound range discussed above. ssiRNA compounds can resemble in length and structure the natural Dicer processed products from long dsiRNAs. Embodiments in which the two strands of the ssiRNA compound are linked, e.g., covalently linked are also included. Hairpin, or other single strand structures which provide the required double stranded region, and a 3′ overhang are also within the invention.

The siRNA compounds described herein, including double-stranded siRNA compounds and single-stranded siRNA compounds can mediate silencing of a target RNA, e.g., mRNA, e.g., an mRNA transcript of a gene that encodes a protein. A gene may also be targeted. In general, the RNA to be silenced is an endogenous gene or a pathogen gene. In addition, RNAs other than mRNA, e.g., tRNAs, and viral RNAs, can also be targeted.

As used herein, the phrase “mediates RNAi” refers to the ability to silence, in a sequence specific manner, a target RNA. While not wishing to be bound by theory, it is believed that silencing uses the RNAi machinery or process and a guide RNA, e.g., an ssiRNA compound of 21 to 23 nucleotides.

In one embodiment, a siRNA compound is “sufficiently complementary” to a target RNA, e.g., a target mRNA, such that the siRNA compound silences production of protein encoded by the target mRNA. In another embodiment, the siRNA compound is “exactly complementary” to a target RNA, e.g., the target RNA and the siRNA compound anneal, for example to form a hybrid made exclusively of Watson-Crick base pairs in the region of exact complementarity. A “sufficiently complementary” target RNA can include an internal region (e.g., of at least 10 nucleotides) that is exactly complementary to a target RNA. Moreover, in certain embodiments, the siRNA compound specifically discriminates a single-nucleotide difference. In this case, the siRNA compound only mediates RNAi if exact complementary is found in the region (e.g., within 7 nucleotides of) the single-nucleotide difference.

Micro RNAs (miRNAs)

“MicroRNAs” (miRNAs or miRs) have been implicated in a number of biological processes including regulation of developmental timing, apoptosis, fat metabolism, and hematopoietic cell differentiation among others. In certain embodiments, miRNAs are small, non-protein coding RNAs of about 18 to about 25 nucleotides in length that regulate gene expression in a sequence-specific manner. miRNAs act as repressors of target miRNAs by promoting their degradation, when their sequences are perfectly complementary, or by inhibiting translation, when their sequences contain mismatches. miRNAs are transcribed by RNA polymerase II (pol II) or RNA polymerase III (pol III; see Qi et al. (2006) Cellular & Molecular Immunology Vol. 3:411-419) and arise from initial transcripts, termed primary miRNA transcripts (pri-miRNAs), that are generally several thousand bases long and are derived from individual miRNA genes, from introns of protein coding genes, or from poly-cistronic transcripts that often encode multiple, closely related miRNAs. See review of Carrington et al. (2003). Pri-miRNAs are processed in the nucleus by the RNase Drosha into about 70- to about 100-nucleotide hairpin-shaped precursors (pre-miRNAs). Following transport to the cytoplasm, the hairpin pre-miRNA is further processed by Dicer to produce a double-stranded miRNA (Lee et al., 1993). The mature miRNA strand is then incorporated into the RNA-induced silencing complex (RISC), where it associates with its target mRNAs by base-pair complementarity. In the relatively rare cases in which a miRNA base pairs perfectly with an mRNA target, it promotes mRNA degradation. More commonly, miRNAs form imperfect heteroduplexes with target mRNAs, affecting either mRNA stability or inhibiting mRNA translation.

Short Hairpin RNAs (shRNAs)

In certain embodiments of the invention, a small hairpin RNA or short hairpin RNA (shRNA) is a sequence of RNA that makes a tight hairpin turn that can be used to silence gene expression via RNA interference. shRNA is transcribed by RNA polymerase III. shRNA production in a mammalian cell can sometimes cause the cell to mount an interferon response as the cell seeks to defend itself from what it perceives as viral attack.

### Ribozymes

According to another embodiment of the invention, targeted mRNA is inhibited by ribozymes, which have specific catalytic domains that possess endonuclease activity (Kim and Cech, Proc Natl Acad Sci USA. 1987 December; 84(24):8788-92; Forster and Symons, Cell. 1987 Apr. 24; 49(2):211-20). For example, a large number of ribozymes accelerate phosphoester transfer reactions with a high degree of specificity, often cleaving only one of several phosphoesters in an oligonucleotide substrate (Cech et al., Cell. 1981 December; 27(3 Pt 2):487-96; Michel and Westhof, J Mol. Biol. 1990 Dec. 5; 216(3):585-610; Reinhold-Hurek and Shub, Nature. 1992 May 14; 357(6374):173-6). This specificity has been attributed to the requirement that the substrate bind via specific base-pairing interactions to the internal guide sequence (“IGS”) of the ribozyme prior to chemical reaction.

At least six basic varieties of naturally-occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nucleic acid molecule may be formed in a hammerhead, hairpin, a hepatitis delta virus, group I intron or RNaseP RNA (in association with an RNA guide sequence) or Neurospora VS RNA motif, for example. Specific examples of hammerhead motifs are described by Rossi et al. Nucleic Acids Res. 1992 Sep. 11; 20(17):4559-65. Examples of hairpin motifs are described by Hampel et al. (Eur. Pat. Appl. Publ. No. EP 0360257), Hampel and Tritz, Biochemistry 1989 Jun. 13; 28(12):4929-33; Hampel et al., Nucleic Acids Res. 1990 Jan. 25; 18(2):299-304 and U.S. Pat. No. 5,631,359. An example of the hepatitis virus motif is described by Perrotta and Been, Biochemistry. 1992 Dec. 1; 31(47):11843-52; an example of the RNaseP motif is described by Guerrier-Takada et al., Cell. 1983 December; 35(3 Pt 2):849-57; Neurospora VS RNA ribozyme motif is described by Collins (Saville and Collins, Cell. 1990 May 18; 61(4):685-96; Saville and Collins, Proc Natl Acad Sci USA. 1991 Oct. 1; 88(19):8826-30; Collins and Olive, Biochemistry. 1993 Mar. 23; 32(11):2795-9); and an example of the Group I intron is described in U.S. Pat. No. 4,987,071. Ribozyme constructs need not be limited to specific motifs mentioned herein.

Methods of producing a ribozyme targeted to any polynucleotide sequence are known in the art. Ribozymes may be designed as described in Int. Pat. Appl. Publ. No. WO 93/23569 and Int. Pat. Appl. Publ. No. WO 94/02595, and synthesized to be tested in vitro and in vivo, as described therein.

Ribozyme activity can be optimized by altering the length of the ribozyme binding arms or chemically synthesizing ribozymes with modifications that prevent their degradation by serum ribonucleases (see e.g., Int. Pat. Appl. Publ. No. WO 92/07065; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

### Supermirs

A supermir refers to a single stranded, double stranded or partially double stranded oligomer or polymer of ribonucleic acid (RNA) or deoxyribonucleic acid (DNA) or both or modifications thereof, which has a nucleotide sequence that is substantially identical to an miRNA and that is antisense with respect to its target. This term includes oligonucleotides composed of naturally-occurring nucleobases, sugars and covalent internucleoside (backbone) linkages and which contain at least one non-naturally-occurring portion which functions similarly. Such modified or substituted oligonucleotides are preferred over native forms because of desirable properties such as, for example, enhanced cellular uptake, enhanced affinity for nucleic acid target and increased stability in the presence of nucleases. In a preferred embodiment, the supermir does not include a sense strand, and in another preferred embodiment, the supermir does not self-hybridize to a significant extent. A supermir can have secondary structure, but it is substantially single-stranded under physiological conditions. A supermir that is substantially single-stranded is single-stranded to the extent that less than about 50% (e.g., less than about 40%, 30%, 20%, 10%, or 5%) of the supermir is duplexed with itself. The supermir can include a hairpin segment, e.g., sequence, preferably at the 3′ end can self-hybridize and form a duplex region, e.g., a duplex region of at least 1, 2, 3, or 4 and preferably less than 8, 7, 6, or n nucleotides, e.g., 5 nucleotides. The duplexed region can be connected by a linker, e.g., a nucleotide linker, e.g., 3, 4, 5, or 6 dTs, e.g., modified dTs. The supermir is duplexed with a shorter oligo, e.g., of 5, 6, 7, 8, 9, or 10 nucleotides in length, e.g., at one or both of the 3′ and 5′ end or at one end and in the non-terminal or middle of the supermir.

### Aptamers

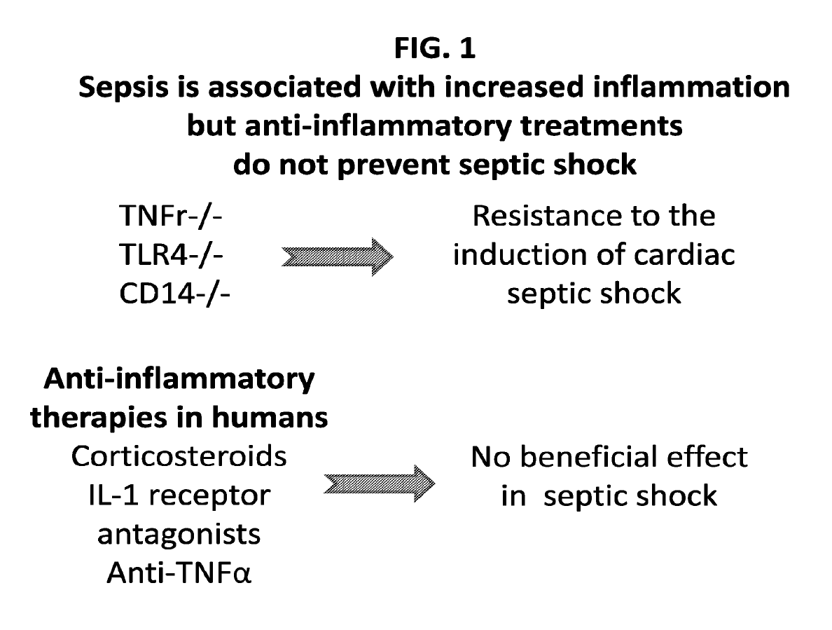

Aptamers are nucleic acid or peptide molecules that bind to a particular molecule of interest with high affinity and specificity (Tuerk and Gold, Science 249:505 (1990); Ellington and Szostak, Nature 346:818 (1990)). DNA or RNA aptamers have been successfully produced which bind many different entities from large proteins to small organic molecules. See Eaton, Curr. Opin. Chem. Biol. 1:10-16 (1997), Famulok, Curr. Opin. Struct. Biol. 9:324-9 (1999), and Hermann and Patel, Science 287:820-5 (2000). Aptamers may be RNA or DNA based, and may include a riboswitch. Regulatory elements are known as riboswitches and are defined as mRNA elements that bind metabolites or metal ions as ligands and regulate mRNA expression by forming alternative structures in response to this ligand binding (FIG. 1; Nudler & Mironov 2004; Tucker & Breaker 2005; Winkler 2005). Although they can bind proteins like antibodies, aptamers are not immunogenic, even at doses up to 1000 times the therapeutic dose in primates.

A riboswitch is a part of an mRNA molecule that can directly bind a small target molecule, and whose binding of the target enables it to regulate its own activity, depending on the presence or absence of its target molecule. Riboswitches are most often located in the 5′ untranslated region (5′ UTR; a stretch of RNA that precedes the translation start site) of bacterial mRNA. There they regulate the occlusion of signals for transcription attenuation or translation initiation. Edwards, A. L. et al., (2010) Riboswitches: A Common RNA Regulatory Element. Nature Education 3(9):9.

Generally, aptamers are engineered through repeated rounds of in vitro selection or equivalently, SELEX (systematic evolution of ligands by exponential enrichment) to bind to various molecular targets such as small molecules, proteins, nucleic acids, and even cells, tissues and organisms. The aptamer may be prepared by any known method, including synthetic, recombinant, and purification methods, and may be used alone or in combination with other aptamers specific for the same target. Further, as described more fully herein, the term “aptamer” specifically includes “secondary aptamers” containing a consensus sequence derived from comparing two or more known aptamers to a given target.

miRNA Mimics

miRNA mimics represent a class of molecules that can be used to imitate the gene silencing ability of one or more miRNAs. In the embodiments of the present invention, miRNA mimics are immunostimulatory agents. Thus, the term “microRNA mimic” refers to synthetic non-coding RNAs (i.e. the miRNA is not obtained by purification from a source of the endogenous miRNA) that are capable of entering the RNAi pathway and regulating gene expression through inhibiting targeted mRNA. miRNA mimics can be designed as mature molecules (e.g. single stranded) or mimic precursors (e.g., pri- or pre-miRNAs) miRNA mimics can be comprised of nucleic acid (modified or modified nucleic acids) including oligonucleotides comprising, without limitation, RNA, modified RNA, DNA, modified DNA, locked nucleic acids, or 2′-O,4′-C-ethylene-bridged nucleic acids (ENA), or any combination of the above (including DNA-RNA hybrids). In addition, miRNA mimics can comprise conjugates that can affect delivery, intracellular compartmentalization, stability, specificity, functionality, strand usage, and/or potency. In one design, miRNA mimics are double stranded molecules (e.g., with a duplex region of between about 16 and about 31 nucleotides in length) and contain one or more sequences that have identity with the mature strand of a given miRNA. Modifications can comprise 2′ modifications (including 2′-O methyl modifications and 2′ F modifications) on one or both strands of the molecule and internucleotide modifications (e.g. phorphorthioate modifications) that enhance nucleic acid stability and/or specificity. In addition, miRNA mimics can include overhangs. The overhangs can consist of 1-6 nucleotides on either the 3′ or 5′ end of either strand and can be modified to enhance stability or functionality. In one embodiment, a miRNA mimic comprises a duplex region of between 16 and 31 nucleotides and one or more of the following chemical modification patterns: the sense strand contains 2′-O-methyl modifications of nucleotides 1 and 2 (counting from the 5′ end of the sense oligonucleotide), and all of the Cs and Us; the antisense strand modifications can comprise 2′ F modification of all of the Cs and Us, phosphorylation of the 5′ end of the oligonucleotide, and stabilized internucleotide linkages associated with a 2 nucleotide 3′ overhang.

### Pharmaceutical Formulations and Administration

The present invention also includes pharmaceutical compositions and formulations of JNK1 and JNK2 inhibitors and of the PPAR agonists, PPARγ agonists, dual PPARα and PPAR γ agonists, or combinations thereof, JNK inhibitors, or antisense nucleic acids, siRNAs, shRNAs, microRNAs (miRNA), ribozymes, microRNA mimics, supermirs, and aptamers that increase PPARα, PPARα-co-activator-1 (PGC-1), estrogen-related receptor (ERR)α, or a combination thereof and other therapeutic agents for treating or preventing cardiac dysfunction, hereafter “the therapeutic agents.” Preferred are pharmaceutical compositions comprising therapeutically effective amounts of one member from at least two of the following groups: (i) JNK1 or a JNK2 inhibitors including antisense nucleic acids, siRNAs, shRNAs, microRNAs (miRNA), ribozymes, microRNA mimics, supermirs, and aptamers; (ii) a PPAR agonists selected from the group comprising PPAR agonists, PPARγ agonists, dual PPARα and PPAR γ agonists; and (iii) PPARα-co-activator-1 (PGC-1) and (iii) estrogen-related receptor (ERR), which amounts treat or prevent cardiac dysfunction in a subject having heart failure or sepsis or at risk of developing them; or in amounts that increase cardiac function also in the patient having heart failure or sepsis or at risk of developing them. Pharmaceutical compositions for use in the present methods include therapeutically effective amounts of one or more of the therapeutic agents, i.e., an amount sufficient to prevent or treat the diseases described herein in a subject, formulated for local or systemic administration. The subject is preferably a human but can be non-human as well. A suitable subject can be an individual who is suspected of having, has been diagnosed as having, or is at risk of developing one of the described diseases, including sepsis-associated cardiac dysfunction associated or other forms of heart failure.

The duration of treatment can extend over several days or longer, depending on the condition, with the treatment continuing until the symptoms of cardiac dysfunction are sufficiently reduced or eliminated. Active agents for therapeutic administration are preferably low in toxicity. Some PPAR agonists have been reported to have toxic effects when administered long term therefore in such cases, administration may be interrupted to balance treating the disease or condition with minimizing toxicity. Treatment of acute cardiac function associated with sepsis is short term, therefore drugs that have long term toxic side effects can be used at the physician's discretion. The progress of this therapy is easily monitored by conventional techniques and assays that may be used to adjust dosage to achieve a desired therapeutic effect.

A composition of the therapeutic agents can also include a pharmaceutically acceptable carrier. As used herein the language “pharmaceutically acceptable carrier” includes solvents, dispersion media, coatings, antiviral agents, antibacterial agents, antifungal agents, isotonic and absorption delaying agents, and the like, compatible with pharmaceutical administration. Supplementary active compounds can also be incorporated into the compositions. Other topical formulations are described in Sheele et al., U.S. Pat. No. 7,151,091.

Therapeutic compositions may contain, for example, such normally employed additives as binders, fillers, carriers, preservatives, stabilizing agents, emulsifiers, buffers and excipients as, for example, pharmaceutical grades of mannitol, lactose, starch, magnesium stearate, sodium saccharin, cellulose, magnesium carbonate, and the like. These compositions typically contain 1%-95% of active ingredient, preferably 2%-70% active ingredient.

The therapeutic agents can also be mixed with diluents or excipients which are compatible and physiologically tolerable as selected in accordance with the route of administration and standard pharmaceutical practice. Suitable diluents and excipients are, for example, water, saline, dextrose, glycerol, or the like, and combinations thereof. In addition, if desired, the compositions may contain minor amounts of auxiliary substances such as wetting or emulsifying agents, stabilizing or pH buffering agents.

In some embodiments, the therapeutic compositions of the present invention are prepared either as liquid solutions or suspensions, or in solid forms. The formulations may include such normally employed additives such as binders, fillers, carriers, preservatives, stabilizing agents, emulsifiers, buffers and excipients as, for example, pharmaceutical grades of mannitol, lactose, starch, magnesium stearate, sodium saccharin, cellulose, magnesium carbonate, and the like. Solutions, suspensions, or sustained release formulations typically contain 1%-95% of active ingredient, preferably 2%-70%.

The formulations may also contain more than one active compound as necessary for the particular indication being treated, preferably those with complementary activities that do not adversely affect each other. Such molecules are suitably present in combination in amounts that are effective for the purpose intended.

Suitable examples of sustained release preparations include semipermeable matrices of solid hydrophobic polymers containing the therapeutic agents, which matrices are in the form of shaped articles, e.g., films, or microcapsule. Examples of sustained release matrices include, but are not limited to, polyesters, hydro gels (for example, poly (2-hydroxyethyl-methacrylate), or poly (vinylalcohol)), polylactides, copolymers of L-glutamic acid and y ethyl-L-glutamate, non-degradable ethylene-vinyl acetate, degradable lactic acid-glycolic acid copolymers such as the LUPRON DEPOT (injectable micro spheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The therapeutic agents of the present invention may be formulated for administration by any suitable means. For in vivo administration, the pharmaceutical compositions are preferably administered orally or parenterally, i.e., intraarticularly, intravenously, intraperitoneally, subcutaneously, or intramuscularly. In particular embodiments, the pharmaceutical compositions are administered intravenously or intraperitoneally by a bolus injection. Stadler, et al., U.S. Pat. No. 5,286,634. For the prevention or treatment of disease, the appropriate dosage will depend on the severity of the disease, whether the drug is administered for protective or therapeutic purposes, previous therapy, the patient's clinical history and response to the drugs and the discretion of the attending physician.

The resulting pharmaceutical preparations may be sterilized by conventional, well known sterilization techniques. The aqueous solutions can then be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc. Additionally, the lipidic suspension may include lipid-protective agents which protect lipids against free-radical and lipid-peroxidative damages on storage. Lipophilic free-radical quenchers, such as α-tocopherol and water-soluble iron-specific chelators, such as ferrioxamine, are suitable.

The pharmaceutical compositions of this invention may be in a variety of forms, which may be selected according to the preferred modes of administration. These include, for example, solid, semi-solid and liquid dosage forms such as tablets, pills, powders, liquid solutions or suspensions, suppositories, and injectable and infusible solutions. The preferred form depends on the intended mode of administration and therapeutic application.

The pharmaceutical compositions of this invention may, for example, be placed into sterile, isotonic formulations with or without cofactors which stimulate uptake or stability. The formulation is preferably liquid, or may be lyophilized powder. For example, the compositions of the invention may be diluted with a formulation buffer comprising 5.0 mg/ml citric acid monohydrate, 2.7 mg/ml trisodium citrate, 41 mg/ml mannitol, 1 mg/ml glycine and 1 mg/ml polysorbate 20. This solution can be lyophilized, stored under refrigeration and reconstituted prior to administration with sterile Water-For-Injection (USP).

Any of the compositions described herein may be comprised in a kit. In a non-limiting example, a PPARα agonist is included in a kit. The kit may further include water and buffer. The kit may also include one or more transfection reagent(s) to facilitate delivery of the agonists to cells. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the agents, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

Such kits may also include components that preserve or maintain the agonists or that protect against their degradation. Such kits generally will comprise, in suitable means, distinct containers for each individual reagent or solution. A kit will also include instructions for employing the kit components as well the use of any other reagent not included in the kit. Instructions may include variations that can be implemented. A kit may also include utensils or devices for administering the agonist by various administration routes, such as parenteral or catheter administration or coated stent.

Suitable Solvates Include Hydrates. Suitable salts include those formed with both organic and inorganic acids or bases. Pharmaceutically acceptable base salts include ammonium salts, alkali metal salts such as those of sodium and potassium, alkaline earth metal salts such as those of calcium and magnesium and salts with organic bases such as dicyclohexylamine and N-methyl-D-glucamine.

Where the PPAR-γ agonist is rosiglitazone, it may be formulated as rosiglitazone maleate. Where the PPAR-γ agonist is pioglitazone, it may be formulated as pioglitazone hydrochloride. Where the PPAR-γ agonist is farglitazar, an exemplary salt form is the sodium salt.

Formulations of use in the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example saline or water-for-injection, immediately prior to use. Extemporaneous injection solutions and suspensions may be prepared from sterile powders, granules and tablets of the kind previously described.

Spray compositions for topical delivery to the lung by inhalation may for example be formulated as aqueous solutions or suspensions or as aerosols delivered from pressurized packs, such as a metered dose inhaler, with the use of a suitable liquefied propellant. Aerosol compositions suitable for inhalation can be either a suspension or a solution of the therapeutic and a suitable.

Medicaments for administration by inhalation desirably have a controlled particle size. The optimum particle size for inhalation into the bronchial system is usually 1-10 μm, preferably 2-5 μm. Particles having a size above 20 μm are generally too large when inhaled to reach the small airways.

It will be understood that the methods and uses of the invention may be employed prophylaxis as well as (more suitably) in the treatment of subjects suffering from sepsis or heart failure.

Therapeutic agents of the present invention may be administered simultaneously meaning the administration of medicaments such that the individual medicaments are present within a subject at the same time. In addition to the concomitant administration of medicaments (via the same or alternative routes), simultaneous administration may include the administration of the medicaments (via the same or an alternative route) at different times.

The invention has been described with reference to specific embodiments. It will, however, be evident that various modifications and changes may be made thereto without departing from the broader spirit and scope of the invention. The specification and drawings are, accordingly, to be regarded in an illustrative rather than a restrictive sense. The invention is illustrated herein by the experiments described above and by the following examples, which should not be construed as limiting. The contents of all references, pending patent applications and published patents, cited throughout this application are hereby expressly incorporated by reference. Although specific terms are employed, they are used as in the art unless otherwise indicated.

### EXAMPLES

### Materials and Methods

Chemical Reagents—

All chemical reagents were obtained from SIGMA.

Animals—

All procedures involving animals were approved by the Institutional Animal Care and Use Committee at Columbia University. Mice were maintained under appropriate barrier conditions in a 12 hr light-dark cycle and received food and water ad libitum. The animals that were used for this study were C57BL/6 mice and mice expressing specifically in cardiomyocytes peroxisome proliferator activating receptor-γ (αMHC-PPARγ) that are also on the C57BL/6 background. All studies were performed on the different genotypes with littermates as controls. Hearts from these mice were harvested, flash frozen and stored at −80° C. until further use. All analyses involving animals were performed with at least 5 mice per experimental group.

Cells—

A human ventricular cardiomyocyte-derived cell line, designated AC-16, was kindly provided by M. M. Davidson.¹²⁶ Cells were maintained in Dulbecco's Modified Eagle Medium: Nutrient Mixture F-12 (Ham) (DMEM:F12) (Invitrogen) supplemented with fetal bovine serum (10%) and a mixture of penicillin and streptomycin (1%). Prior to infection with recombinant adenoviruses the medium was changed to 2% heat inactivated horse serum and the cells were infected in at least duplicates with control adenovirus that expresses the Green Fluorescent Protein (Ad-GFP) or the adenovirus expressing the constitutively active form of JNK2 (Ad-JNK2α2) at a multiplicity of infection (MOI) of 10. Sixteen hours post-infection, cells were washed with phosphate-buffered saline, and fresh 10% fetal bovine serum containing medium was added. To assess gene expression 48 hours post incubation, cell lysates were collected and analyzed for mRNA and protein expression.

Construction of Recombinant Adenovirus Expressing a Constitutively Active Form of JNK2—

The pEGFP-C1-JNK2α2 plasmid that contained the cDNA of the constitutively active JNK2 (JNK2α2)¹²⁷ was kindly provided by Albert J. Wong, MD (Stanford University Medical Center, CA). The JNK2α2 cDNA was isolated by digestion with XhoI and BamHI and was initially cloned in the pcDNA3.1 plasmid. Double digestion with XhoI and HindIII was then applied to the pcDNA3.1-JNK2α2 to isolate the JNK2α2 cDNA and clone it in the pAdTrack-CMV plasmid. The pAdTrack-CMV-JNK2α2 plasmid was used to generate recombinant adenovirus as described previously.¹²⁸

LPS-Mediated Induction of Sepsis—

LPS (5 mg/kg) (Sigma) was administered intraperitoneally (i.p.) in mice. Control mice were treated with equal volume of saline. Cardiac function was assessed by 2D echocardiography 5-6 hours post-LPS administration and mice were sacrificed 2-3 hours later (7-9 hours post-LPS injection).

Echocardiography—

Two-dimensional echocardiography was performed on conscious 10 to 12 week-old male and female (n=5-8 per group) mice (Sonos5500 system; Philips Medical Systems).¹²⁹Echocardiographic images were recorded in a digital format. Images were then analyzed off-line by a single observer blinded to the murine genotype.¹³⁰

RNA Purification and Gene Expression Analysis—

Total RNA was purified from cells or hearts using the TRIzol reagent according to the instructions of themanufacturer (Invitrogen). cDNA was synthesized using the SuperScript III First-Strand Synthesis SuperMix (Invitrogen). cDNA was analyzed with quantitative real-time PCR that was performed with SYBR Green PCRCore Reagents (Stratagene). Incorporation of the SYBRgreen dye into the PCR products was monitored in real time withan Mx3000 sequence detection system (Stratagene). Samples were normalized against β-actin.

Protein Purification and Analysis—

Isolated heart tissues or cells were homogenized in PBS containing protease inhibitors and phosphatase inhibitors (Roche, Indianapolis, Ind.). Membrane and cytosolic fractions were separated by ultracentrifugation.20 μg from each fraction was applied to SDS-PAGE and transferred onto nitrocellulose membranes. Antibodies were obtained from Santa Cruz (JNK) and Cell Signaling (phospho-JNK, phospho-AMPK^{Thr 172} and total AMPK).

FA Oxidation—

FA oxidation was measured in pieces of hearts isolated from 10-12 week old mice. The heart pieces were incubated at 37° C. for 2 h in modified Krebs-Ringerbuffer (MKR: 115 mM NaCl, 2.6 mM KCl, 1.2 mM KH₂PO₄, 10 mM NaHCO₃,10 mM HEPES, pH 7.4) that contained 2% BSA, 0.2 mmol/ml palmitate and 10 μCi/ml 9,10(n)-[³H]-palmitate and was gassed with 95% O₂ and 5% CO₂. Water was then extracted with chloroform:methanol (2:1) extraction. Palmitate oxidation was determined by measuring the amount of ³H₂O in the aqueous phase.

Cardiac ATP Measurement—

Cardiac pieces of 10 mg were used to determine ATP levels. Heart pieces were dissolved in ice-cold 0.1% trichloro-acetic acid and centrifuged. Supernatant was resuspended in 50 mM Tris-acetate containing 2 mM EDTA, pH 7.8 and a portion was used to measure ATP. The measurement was performed with the ATP determination kit (Invitrogen) according to the instructions of the manufacturer.

Electron Microscopy—

Left ventricles from 3-month-old mice were fixed with 2.5% glutaraldehyde in 0.1 M Sorensen's buffer (0.2 M monobasic phosphate/0.2 M dibasic phosphate, 1:4 vol/vol; pH 7.2), postfixed in osmium tetroxide, and embedded in EPON 812 (Electron Microscopy Sciences). Ultrathin sections were stained with uranyl acetate and lead citrate and examined under a JEM-1200ExII electron microscope (JEOL).

Assessment of Survival—

C57BL/6 mice were challenged in groups of 10-14 with a lethal dose of LPS (15 mg/kg) by i.p. injection and were observed for survival for 72 h. The effect of rosiglitazone on LPS-induced mortality was assessed by giving i.p. doses of rosiglitazone (35 mg/kg/day). Control animals received saline and DMSO instead of LPS and rosiglitazone, respectively.

Statistical Analysis—

Comparisons between 2 groups were performed using unpaired 2-tailed Student's t tests. All values are presented as mean±SE. Differences between groups were considered statistically significant at P<0.05.

PPAR agonists that can be used to prevent or treat cardiac dysfunction associated with sepsis include, but are not limited, the following:

| | | PPARα/ |
|---|---|---|
| | | PPARγ dual |
| PPARγ agonists | PPARα agonists | agonists |
| thiazolidinedione | Alpha WY-14643 | MK-767/KRP-297 |
| rosiglitazone (SKB) | GW9578 | (Merck/Kyorin) |
| pioglitazone (Takeda) | GW-590735 | E966-E971 |
| Mitsubishi's MCC-555 | K-111 | AZ-242 |
| (disclosed in U.S. Pat. | LY-674 | tesaglitazar; |
| No. 5,594,016) | KRP-101 | Astra-Zeneca |
| Glaxo-Welcome's GL- | DRF-10945 | muraglitazar; |
| 262570 | LY518674 | and certain |
| englitazone (CP-68722, | Propanoic Acid | compounds |
| Pfizer) | 2-[4-[3-[2,5-dihydro-1- | described |
| darglitazone (CP-86325, | [(4- | in U.S. Pat. No. |
| Pfizer | methylphenyl)methyl]-5- | 6,414,002. |
| isaglitazone (MIT/J&J) | oxo-1H-1,2,4-triazol-3-- | |
| JTT-501 (JPNT/P&U) | yl]propyl]phenoxy]-2- | |
| T-895645 (Merck) | methyl, fibrate | |
| R-119702 (Sankyo/WL) | fenofibrate | |
| N,N-2344 (Dr. | clofibrate | |
| Reddy/NN) | bezafibrat | |
| YM-440 (Yamanouchi | | |
| GI 262570 | | |
| R-483 and | | |
| CS-011 (rivoglitazone) | | |

Other PPAR agonists include GW 9578NN622/ragaglitazar, BMS 298585, BRL49634, KRP-297, JTT-501, SB 213068, GW 1929, GW 7845, GW 0207, L-796449, L-165041 and GW 2433.

Heart failure that is not caused by or associated with acute sepsis can also be treated with the methods described above for increasing FAO in cardiomyocytes by administering therapeutically effective amounts of PPARα and or PPARγ agonists or JNK inhibition, preferably those that do not have long term adverse side effects. However agents that have unacceptably high toxicity over the long term could be used for relative brief periods to treat heart failure.

### Example 1

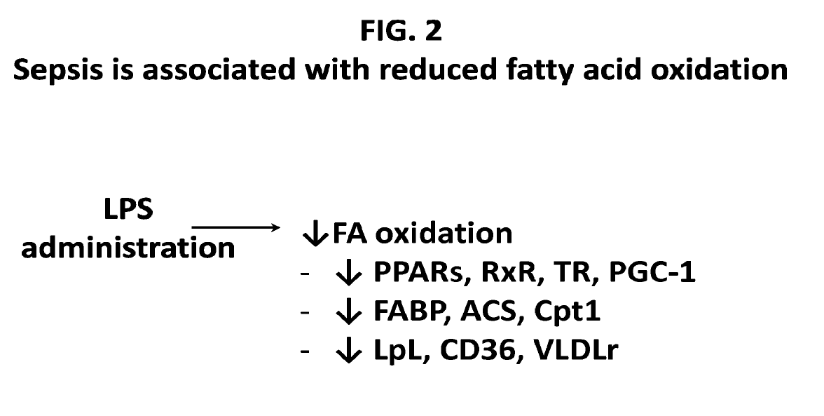

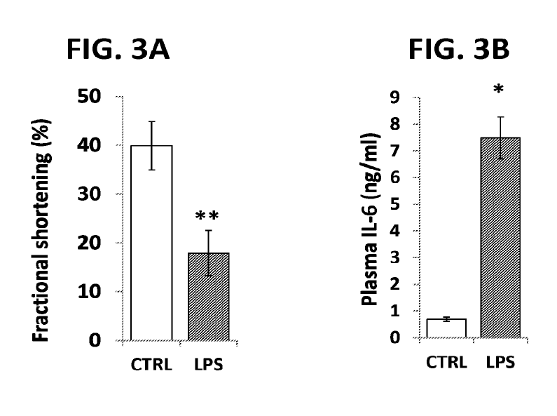

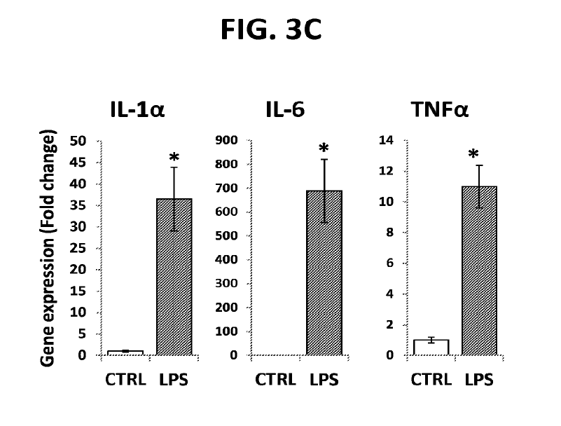

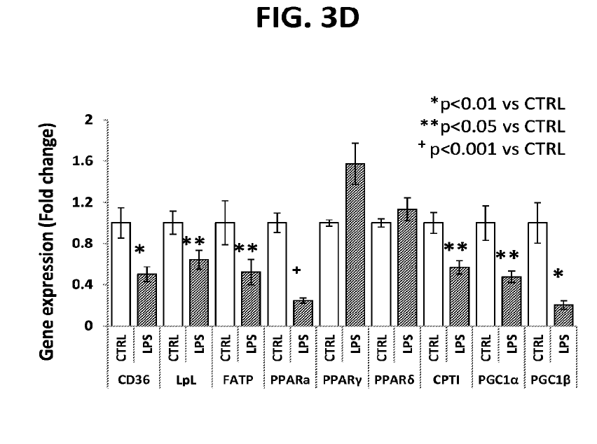

An animal model of cardiac septic shock was reproduced by treating 10 week old C57BL6 mice with LPS (5 mg/kg of body) for 6-8 h. Consistently with previous studies,^{154,165} LPS reduced fractional shortening (FIG. 3(A) and mRNA levels for cardiac PPARα, CD36, LpL, FATP, Cpt1β, PGC-1α and PGC-1β (FIG. 3(D)). These changes were associated with increased cardiac gene expression of inflammation-associated genes (IL-1^{α}, IL-6 and TNFα) and plasma IL-6 protein levels (FIG. 3(B)-(C)). Cardiac ATP was reduced by 44% in LPS-treated mice and FAO was reduced by 61%. Thus, the model is valid for cardiac septic shock. FIG. 2.

### Example 2

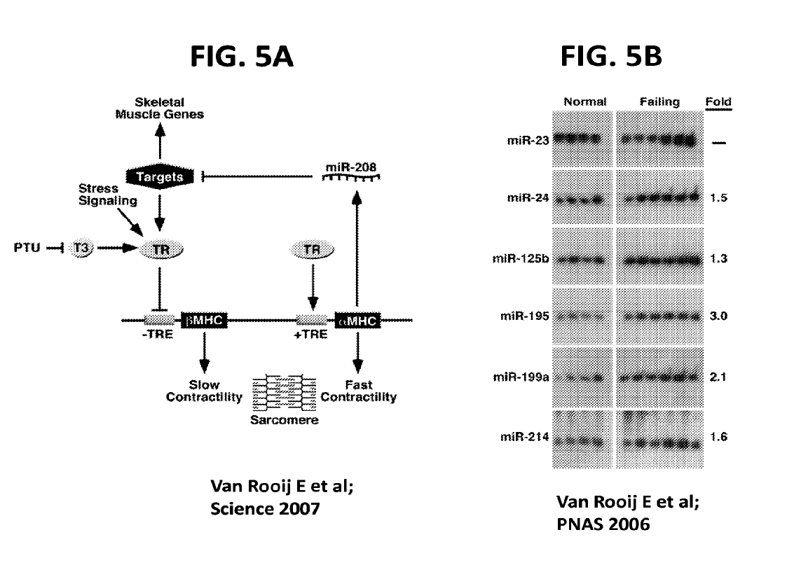

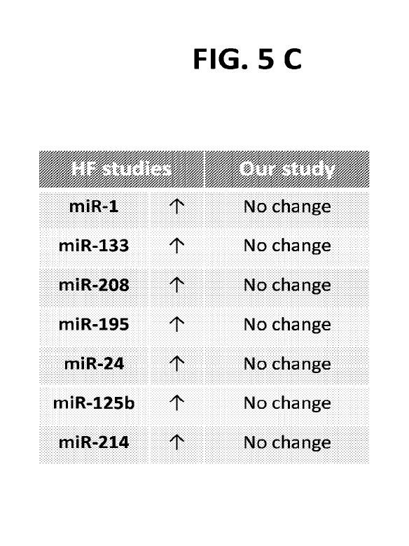

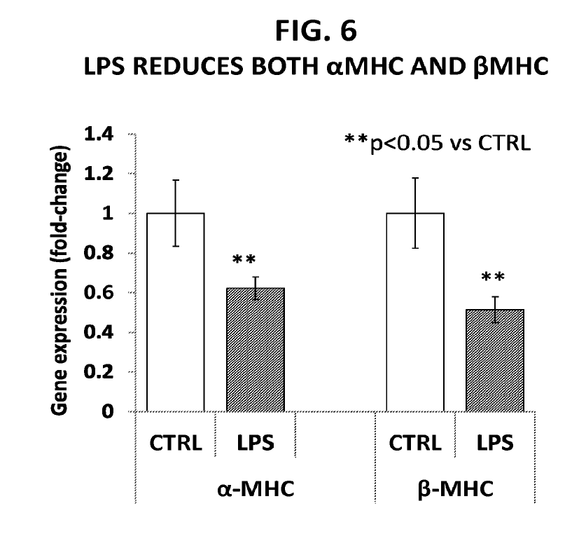

The next experiment assessed whether the cardiac miR (microRNA) profiles of LPS-treated mice resemble the respective profiles in heart failure. (FIG. 5(A)-(C)). MiR array analysis of cardiac RNA from LPS-treated mice showed that none of the miRs that have been associated with heart failure, [miR-1, -133, -208, -195, -24, -125b, -199a and 214¹⁴⁹⁻¹⁵¹], was affected (FIG. 5 (C)). Another indication of heart failure is the switch from the α-MHC isoform to β-MHC.^{166, 167} Cardiac mRNA analysis did not indicate such change (FIG. 6) in the model, so the mechanism of LPS-mediated reduction in FAO is distinct from that which occurs with afterload induced heart failure.

### Example 3

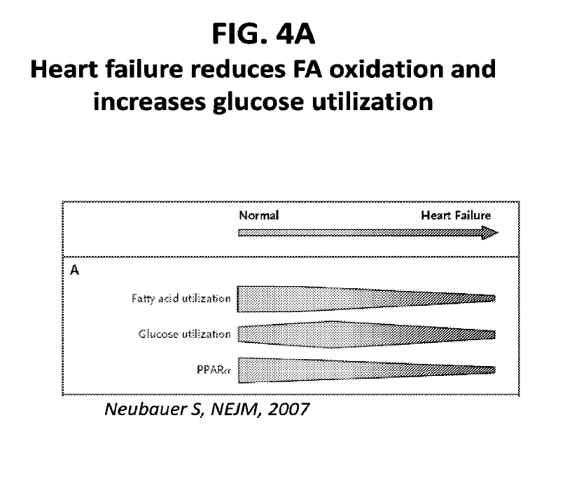

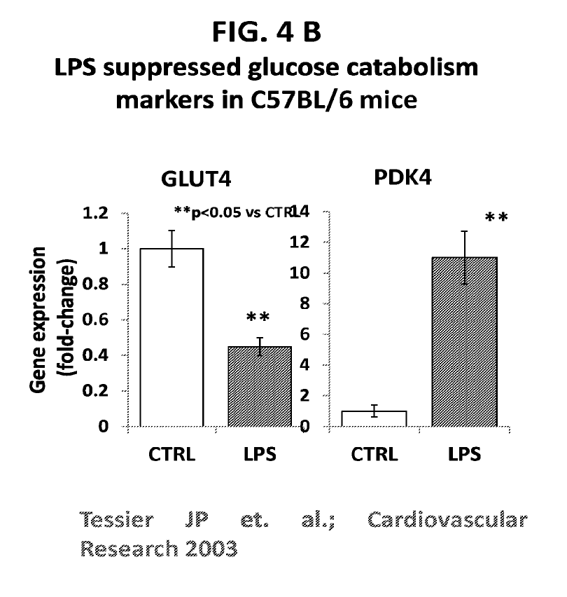

Sepsis following Gram negative (−) bacterial infection is associated with both elevated inflammation (FIG. 1) and reduced FAO (FIG. 2). Animals that are knock-out for inflammation-related genes¹³³⁻¹³⁹ are resistant to LPS-mediated sepsis and have improved cardiac function or viability. However, anti-inflammatory drugs, although treatment of inflammation may be protective in the early stage of sepsis, has not improved mortality.¹⁴⁰⁻¹⁴⁵ LPS treatment leads to reduced expression of PPARα and other FA metabolism related-genes¹⁴⁶⁻¹⁴⁸ (FIG. 3). LPS also suppresses glucose catabolism. FIG. 4(B) Heart failure reduces FAO and increases glucose utilization FIG. 4(A). Several miRs have been linked to heart failure¹⁴⁹⁻¹⁵¹ that are also associated with impaired FAO, however, none of these miRs are significantly altered in the hearts of LPS-treated mice. (FIG. 5) Thus, mechanisms that reduce FAO exclusive of miR-induced changes are ignited by LPS treatment.

### Example 4

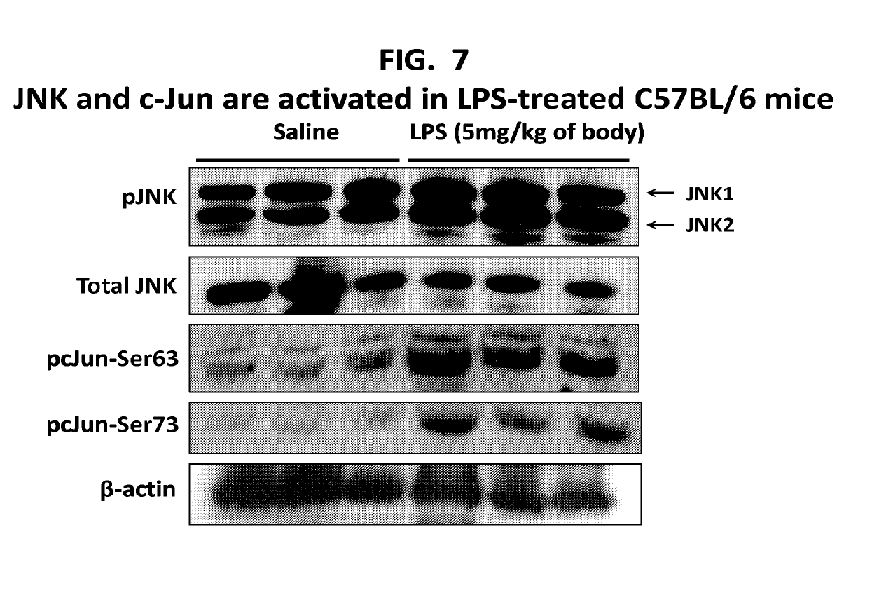

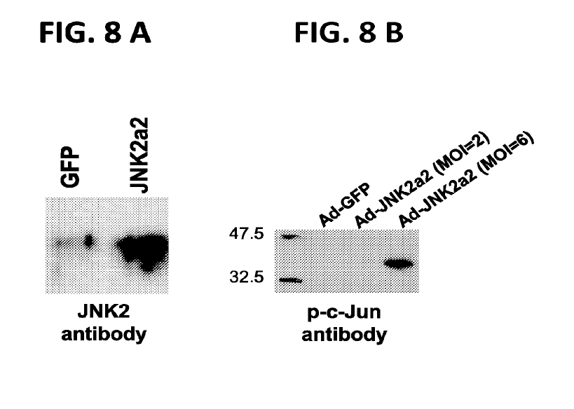

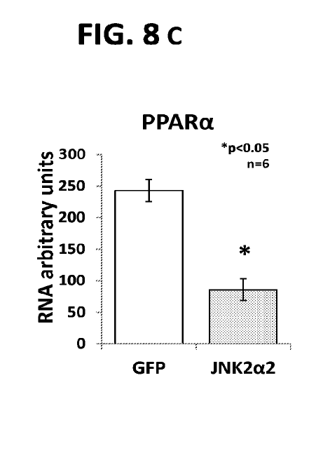

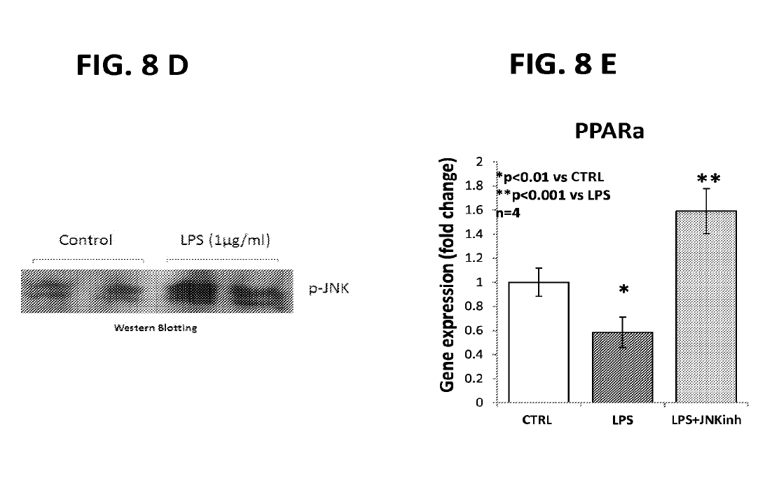

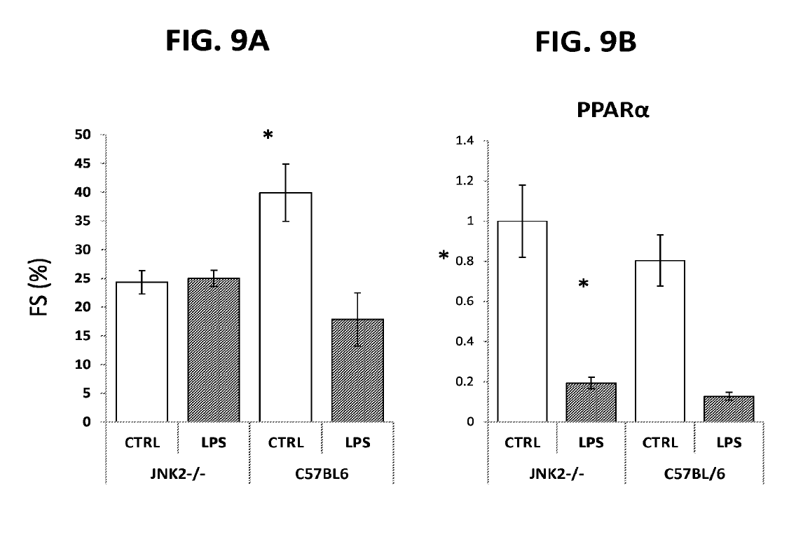

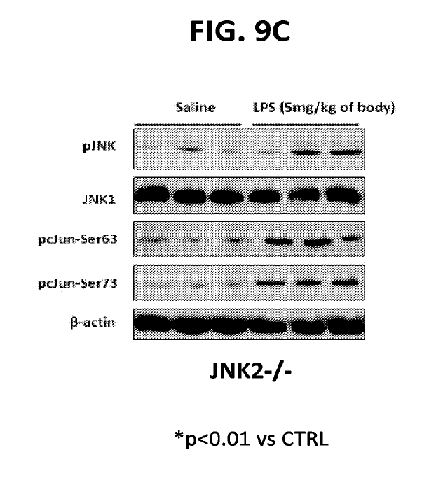

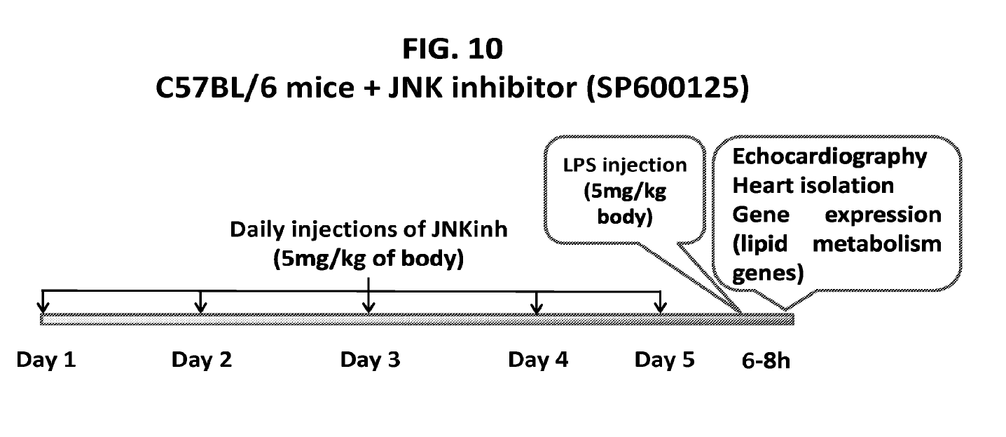

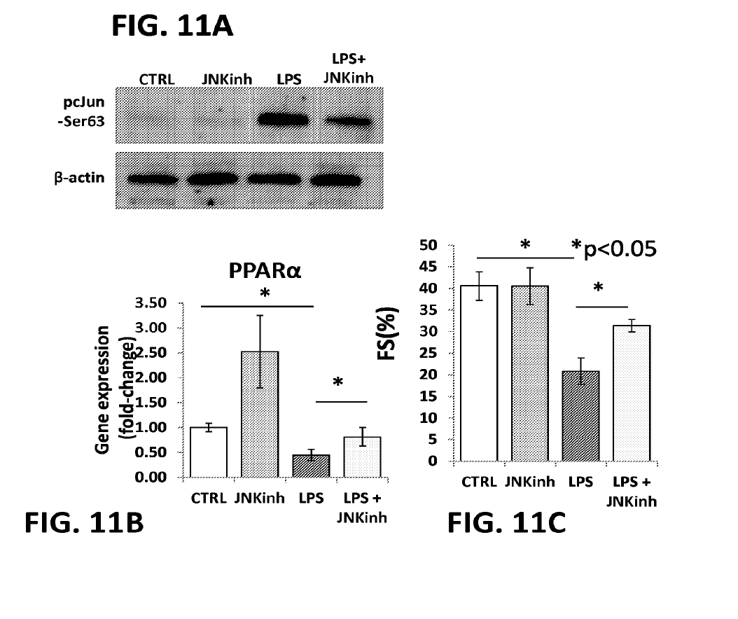

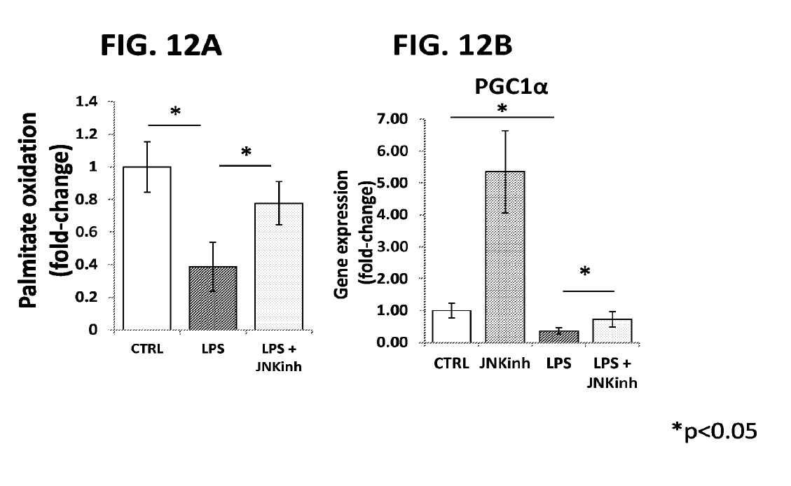

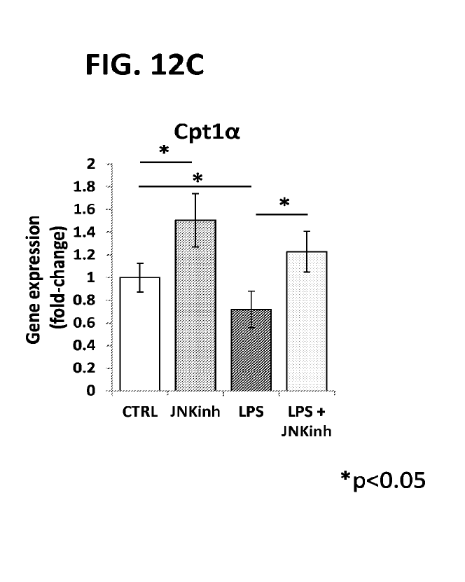

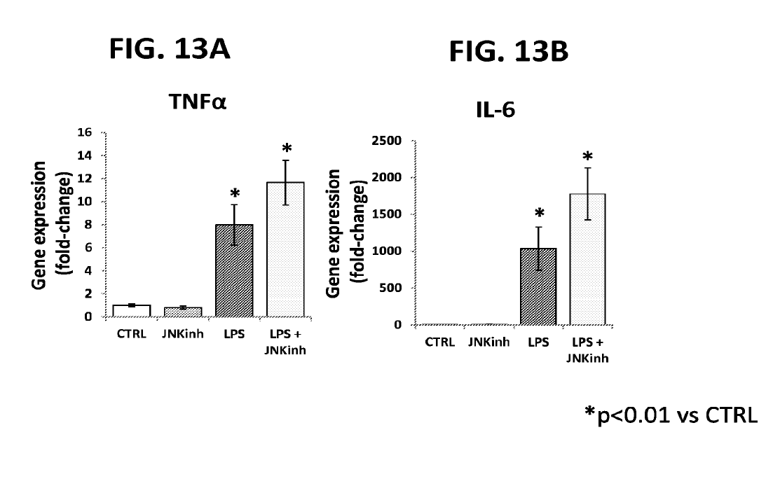

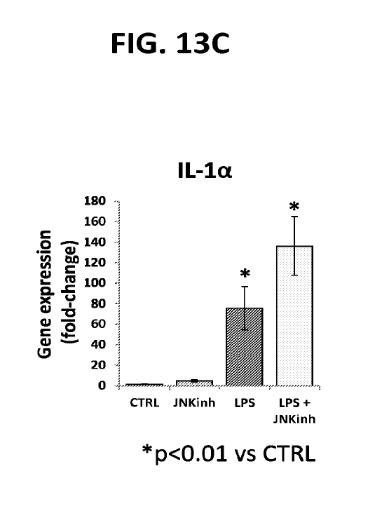

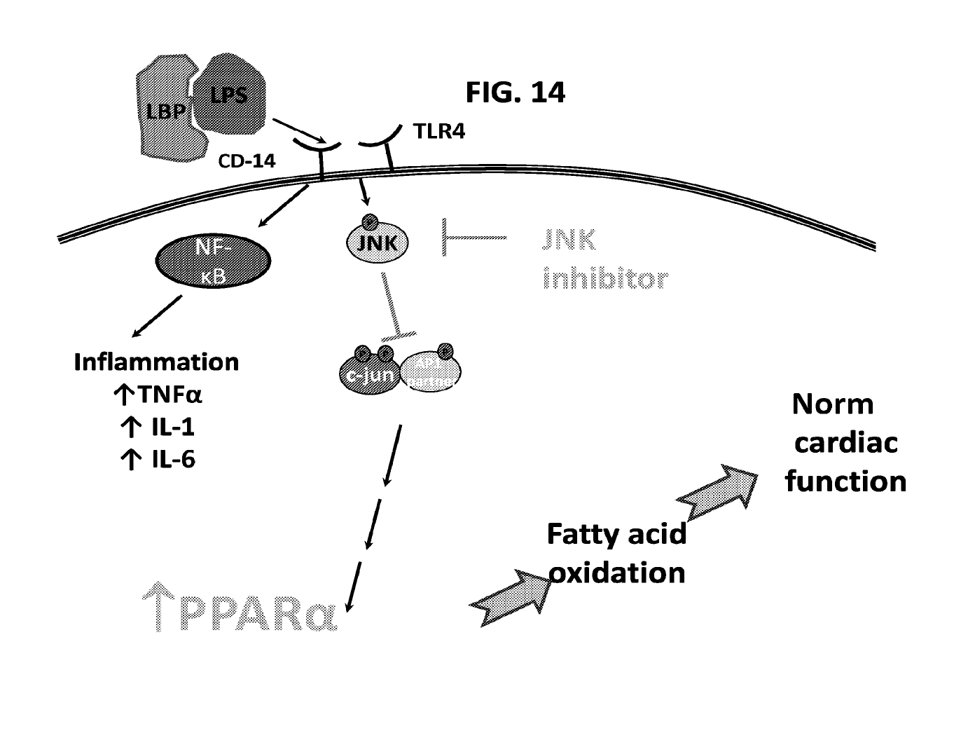

Cardiac function in normal C57BL/6 mice was dramatically improved by administering the JNK inhibitor SP600125, which increased FAO, even though there was no decrease in inflammation. FIGS. 7-14. In vivo and in vitro experiments show that JNK-mediated activation of c-Jun is needed for cardiac PPARα downregulation. The results in FIG. 8(A)-(E) show that constitutively active JNK2 reduced PPARα expression in vitro in a human cardiomyocyte cell line. LPS did not affect cardiac function which is already reduced in JNK2−/− mice. FIG. 9(A). But LPS dramatically reduced PPARα gene expression in JNK2−/− mice and in normal mice. FIG. 9 (B)-(C). It has now been discovered that inhibiting JNK by administering JNK inhibitor SP600125 to LPS-treated septic C57BL/6 mice within 24 hours after LPS administration increased PPARα, blocked the LPS-induced reduction of PPARα gene expression and normalized cardiac function (measured as % of fractional shortening (FS)) FIG. 11(A)-(C). That these effects were associated with increased FAO is shown by the normalization of (FIG. 12(A)) palmitate oxidation and gene expression of carnitine palmitoyl transferase type 1 alpha (CPT-1alpha) (FIG. 12(C), a key enzymein mitochondrial fatty acid catabolism. FIG. 12 (A)-(C). This increase in FAO occurred even though inflammatory cytokine markers were elevated (FIG. 13(A)-(C)), supporting the discovery that in sepsis the inflammatory pathway and the FAO pathway that is associated with cardiac function are separate. FIG. 14.

Therefore certain embodiments of the invention are directed to a method for increasing or maintaining cardiac function in a subject who has or is at risk of developing sepsis or heart failure, by administering therapeutically effective amounts of an agent that increases fatty acid oxidation in the heart. In a preferred embodiment, the agent is a PPARα agonist or a JNK inhibitor such as SP600125, or a combination thereof. Another embodiment is directed to treating cardiac dysfunction by administering an antisense nucleic acid, siRNA or shRNA, micro RNA (miRNA), ribozyme, microRNA mimics, supermir, and aptamer that specifically hybridizes to JNK thereby reducing its expression alone or with PPARα agonists. PPARα agonists for use in the present invention are listed above in the Materials and Methods section.

### Example 5

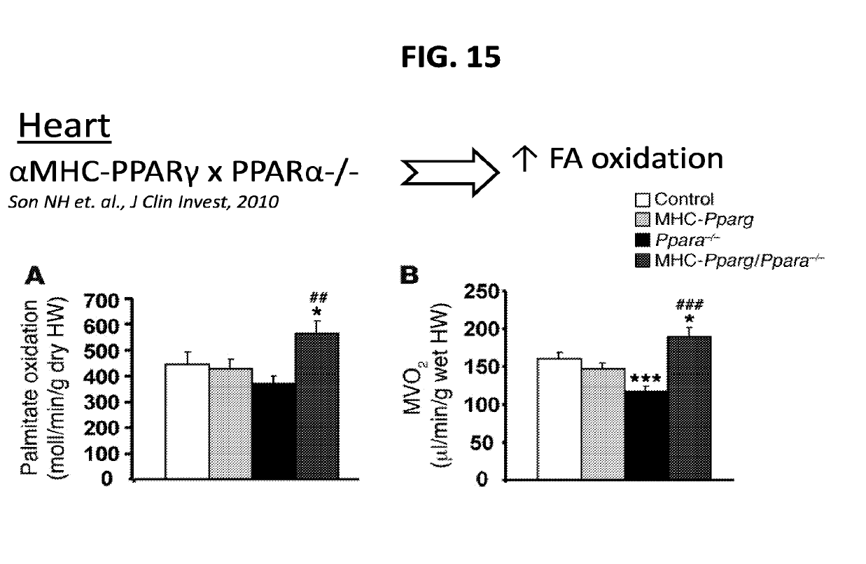

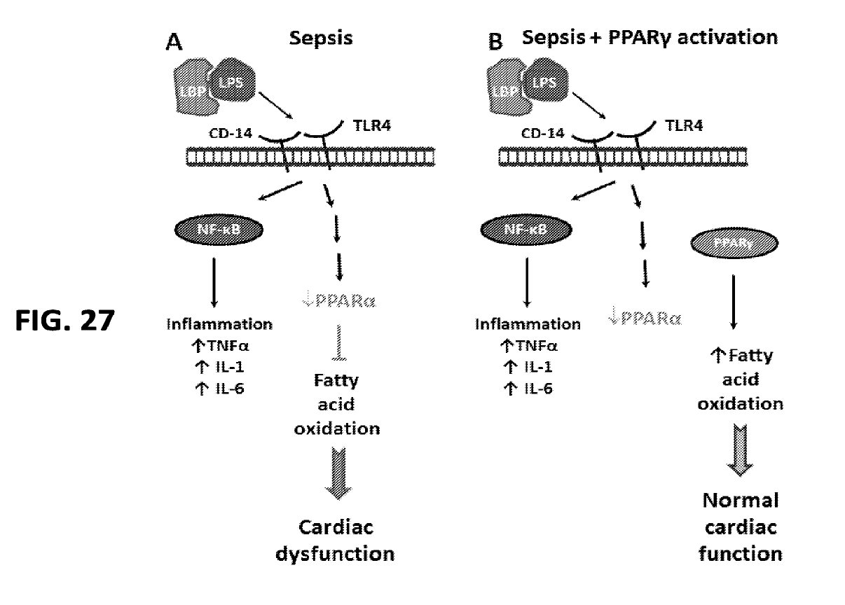

The LPS-mediated reduction in FAO in normal mice can be explained by a reduction of cardiac PPARα.;^{148, 154} however, PPARγ levels increased by ˜60%. The heart expresses all three members of the PPAR family of nuclear transcription factors, each of which has been shown in vivo to activate pathways required for FAO.¹⁵⁵⁻¹⁵⁹ To determine the role of PPARγ during sepsis, PPARα knockout transgenic mice that constitutively express cardiac-specific PPARγ(αMHC-PPARγ/PPARα^{-/-}) were induced to develop sepsis by administering LPS. αMHC-PPARγ/PPARα^{-/-} mice showed elevated FAO levels compared to both αMHC-PPARγ and PPARα−/− mice.¹⁵⁹ (FIG. 15). The role of PPARγ is shown in a schematic model that explains the role of the activation of PPARγ in the induction of fatty acid oxidation and prevention of LPS-mediated cardiac dysfunction. FIG. 27.

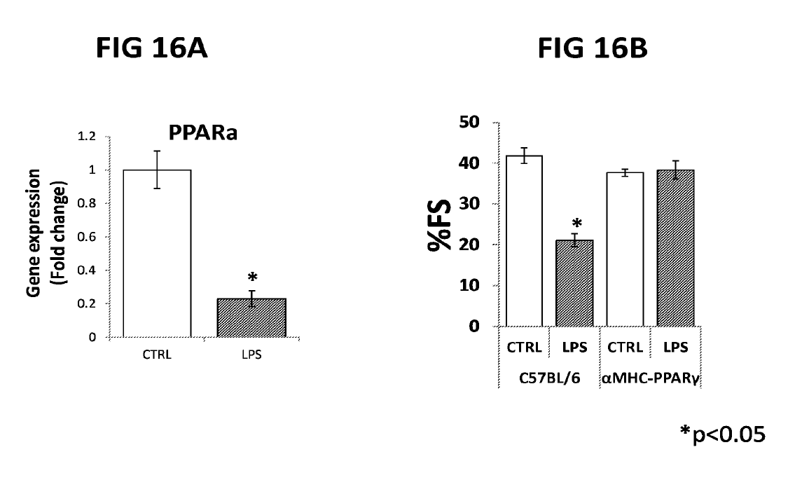

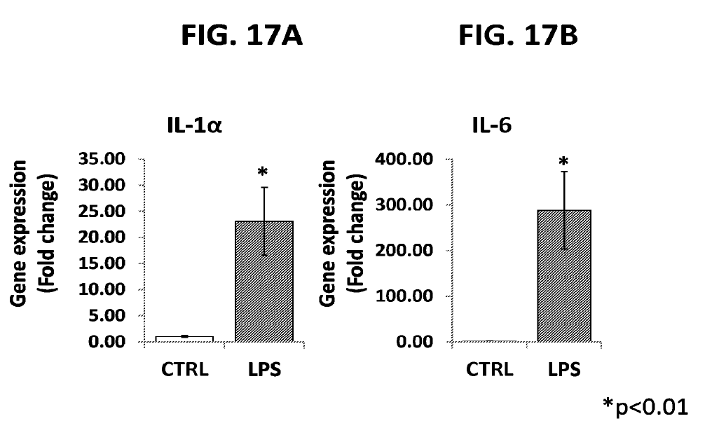

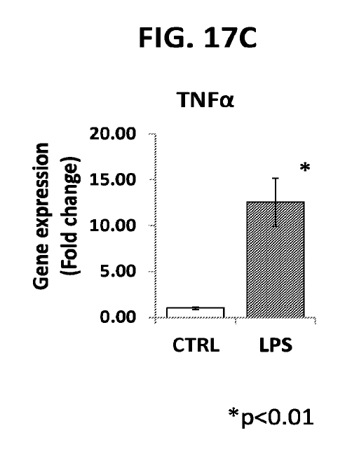

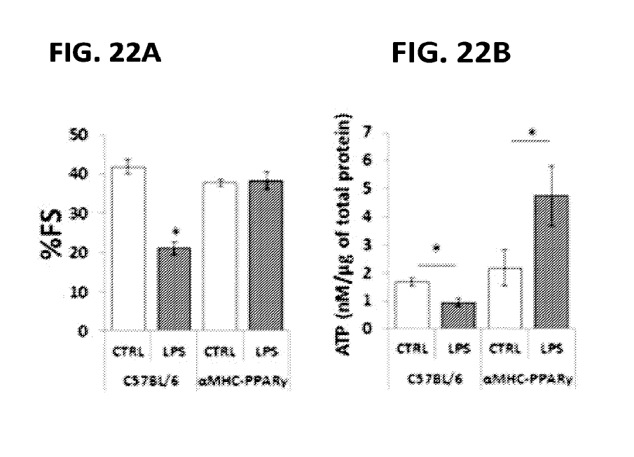

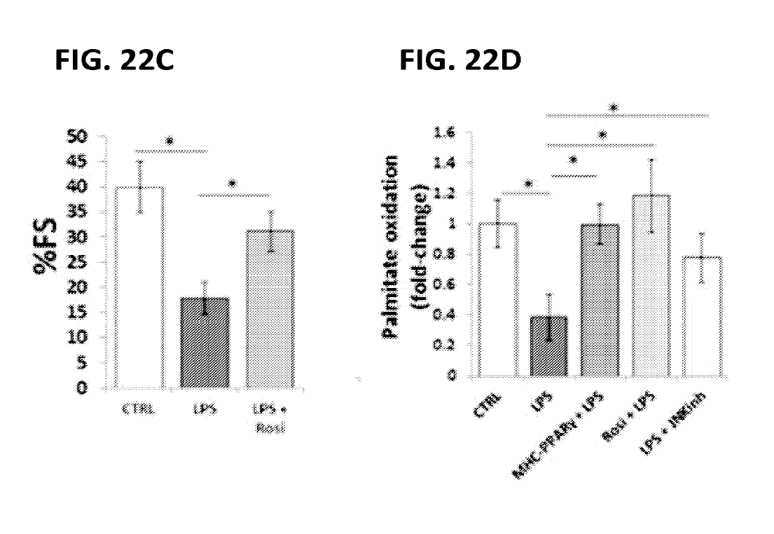

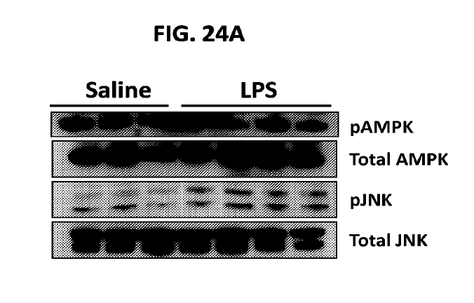

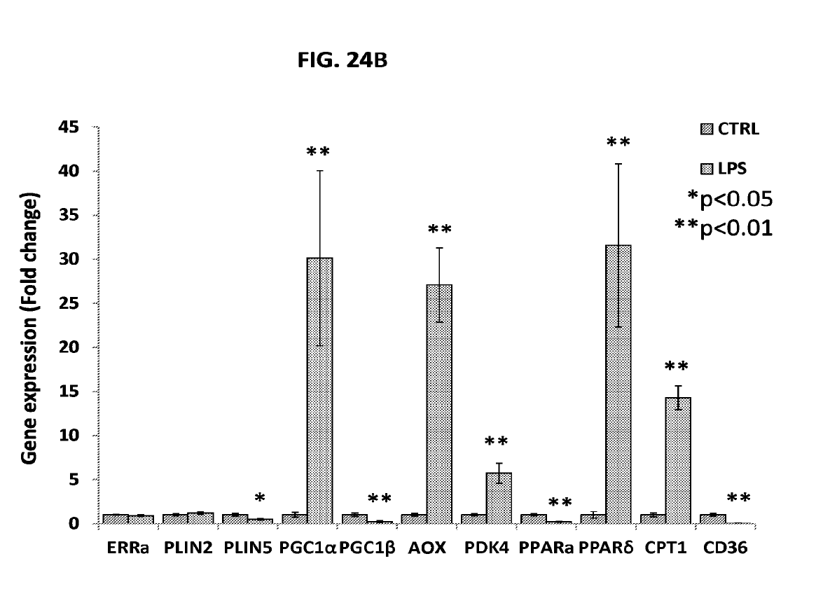

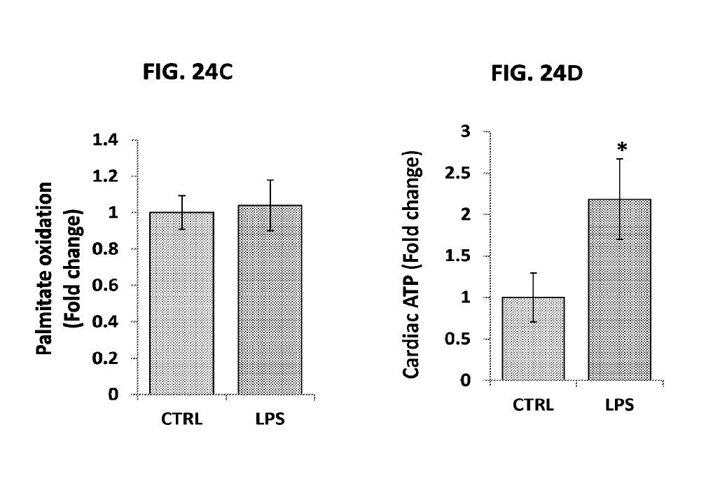

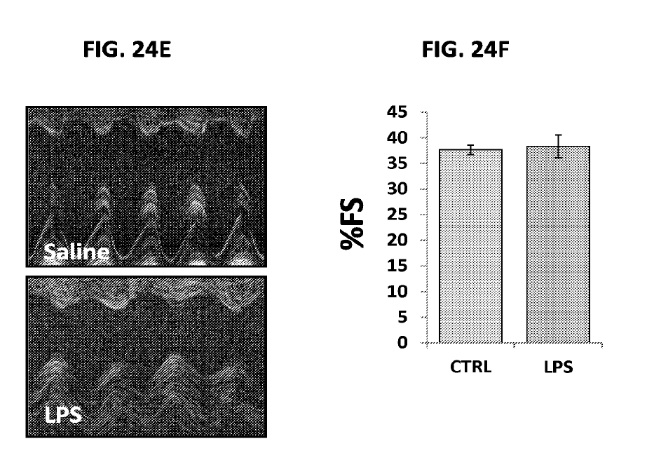

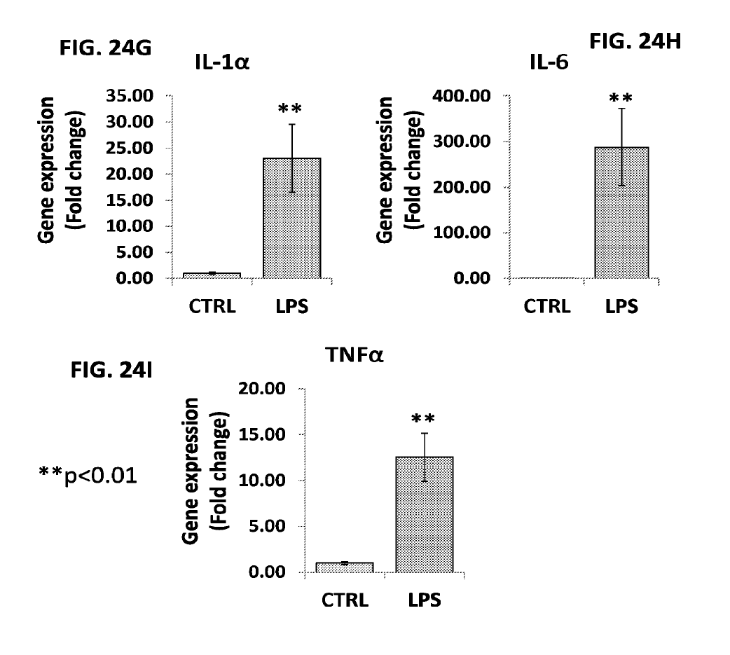

Unexpectedly, constitutive expression of PPARγ in PPARα knockout transgenic mice protected against LPS-induced cardiac dysfunction despite PPARα downregulation in αMHC-PPARγ mice. FIG. 16 (A)-(B). Despite a 77% reduction in PPARα, the expression of FAO-associated genes (such as AOX, PGC-1α, PPARδ, Cpt1β, UCP2 and UCP3) increased by 27.1-, 30.1-, 31.6-, 14.3-, 23.7- and 17.4-fold, respectively FIG. 24(B). Similarly, cardiac ATP content was also significantly increased. FIG. 22(B). As expected, LPS increased the inflammation-related gene expression of IL-1α, FIG. 24(G); IL-6 FIG. 24(H); and TNFα FIG. 24(I) by 23.1-, 288- and 12.5-fold, respectively showing that cardiac function of LPS-treated αMHC-PPARγ mice remained normal despite elevated inflammation. See also FIG. 17(A)-(C).

PPARα and/or PPARγ agonists, pharmaceutically acceptable salts, solvates, solvates of such salts or prodrugs thereof are well known in the art and many of them are FDA-approved drugs. The PPARγ agonists rosiglitazone, troglitazone and pioglitazone are thiazolidine derivatives that are well known insulin sensitizers, prescribed to ameliorate insulin resistance (or enhance the action of insulin) and lower blood glucose without promoting secretion of insulin from the pancreas. Thiazolidine-type chemicals induce differentiation of adipocytes, and exhibit their action via the intranuclear receptor PPARγ¹⁹⁸ for which they act as agonists.¹⁹⁹Unfortunately, some PPARγ agonists particularly, rosiglitazone (that has recently been pulled from the market) and troglitazone, cause hepatic damage with long term use. Short term use of these drugs for treating acute sepsis, however, will not have toxicity issues.

### Example 6

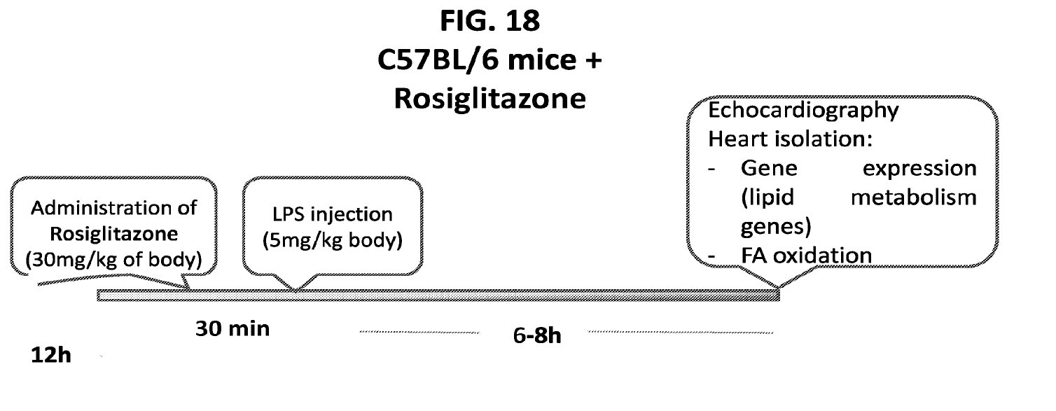

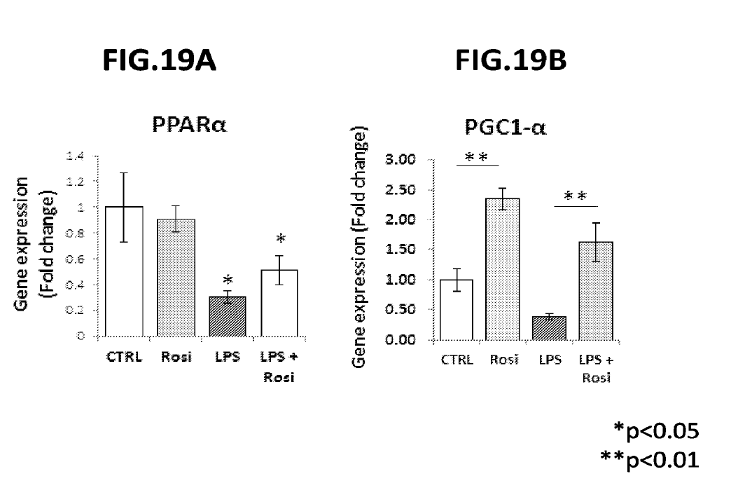

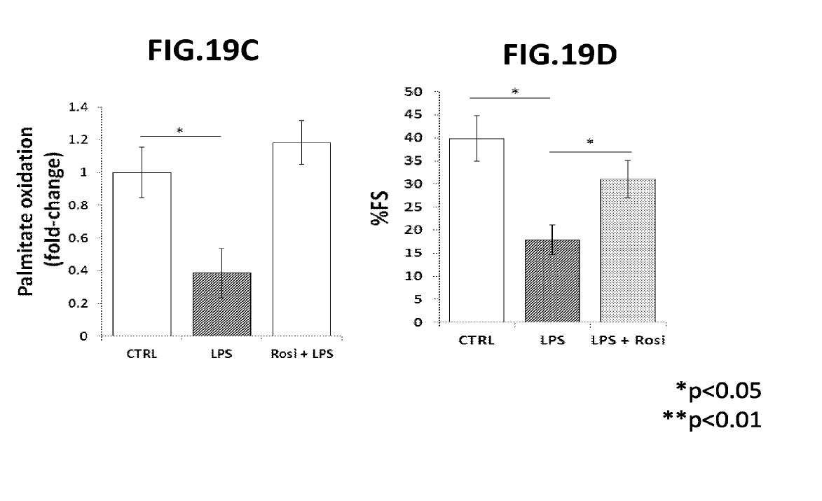

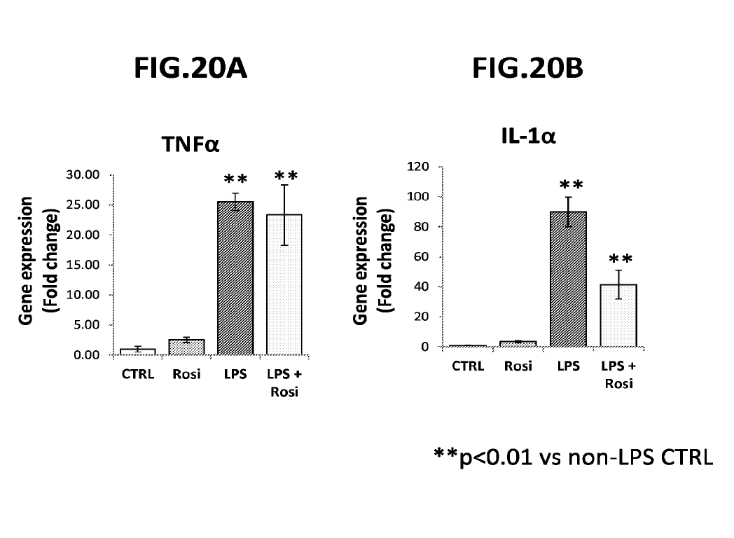

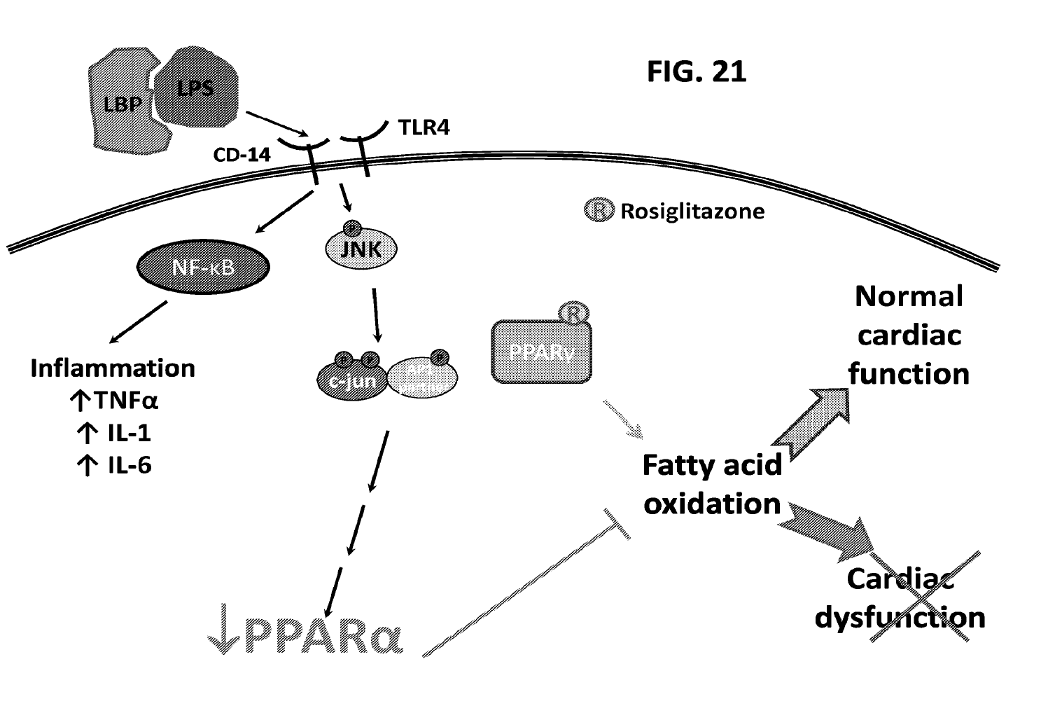

C57BL/6 mice were treated with LPS and the PPARγ agonist, rosiglitazone intraperitoneally (i.p.) (30 mg/kg) to determine whether pharmacologic activation of PPARγ would have the same effects on normal mice that were observed with αMHC-PPARγ mice. FIG. 18. Rosiglitazone increased various indicia of FAO in normal LPS-treated mice: PGC1-α expression was increased about 75% above control values; palmitate oxidation increased about 20%; and cardiac function measured as % FS was restored to about 80% of normal levels. FIGS. 19(A)-(D), 22(C) and 22(D). It is important to note that the LPS-mediated downregulation of PPARα mRNA and upregulation of the inflammatory cytokines IL-1α, IL-6 and TNFα was not reversed by rosiglitazone treatment. Thus, activation of PPARγ in normal LPS-treated mice by rosiglitazone prevented cardiac dysfunction, increased cardiac FAO gene expression and stabilized ATP content, even though the mice displayed concurrent cardiac inflammation and PPARα downregulation. As expected, inflammation was not elevated by the PPARγ agonist R treatment (FIG. 20(A)-(B)) and cardiac function was normalized in LPS-treated C57BL/6 mice. These results show that pharmacological-mediated activation of PPARγ in septic mice compensated for PPARα downregulation, boosted energy production and prevented cardiac dysfunction.

### Example 7

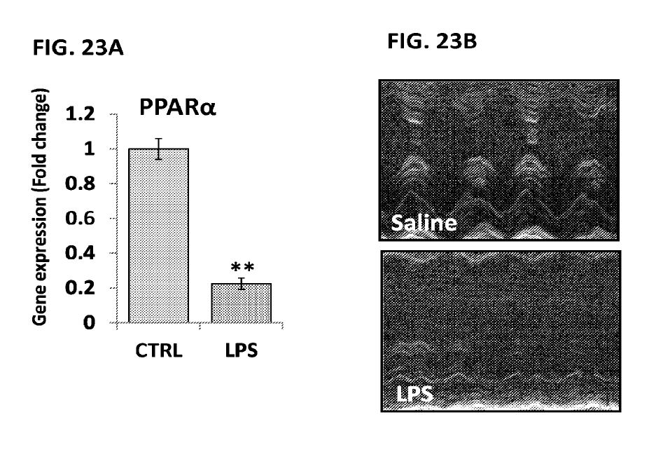

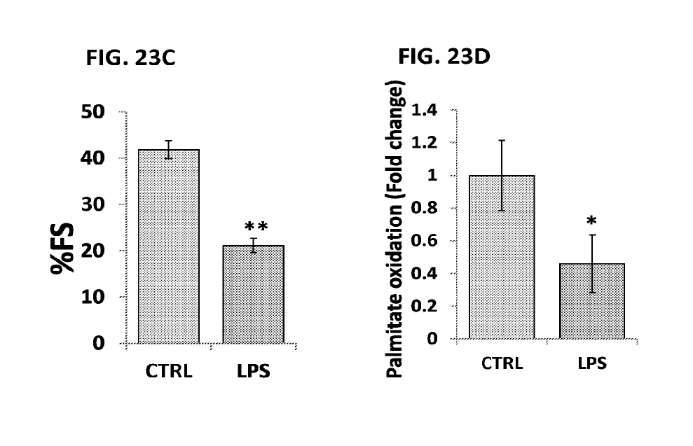

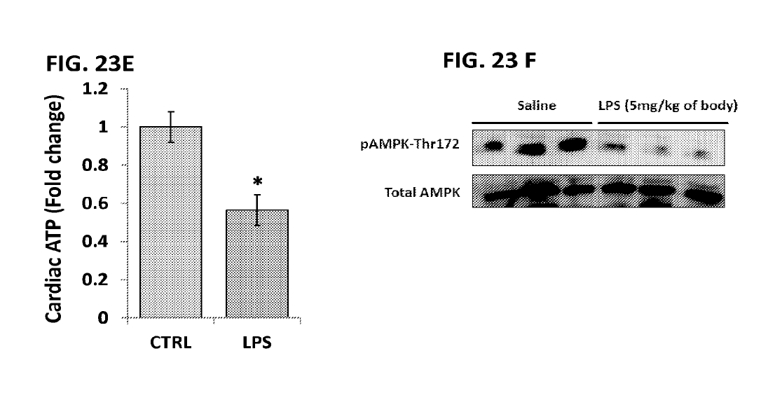

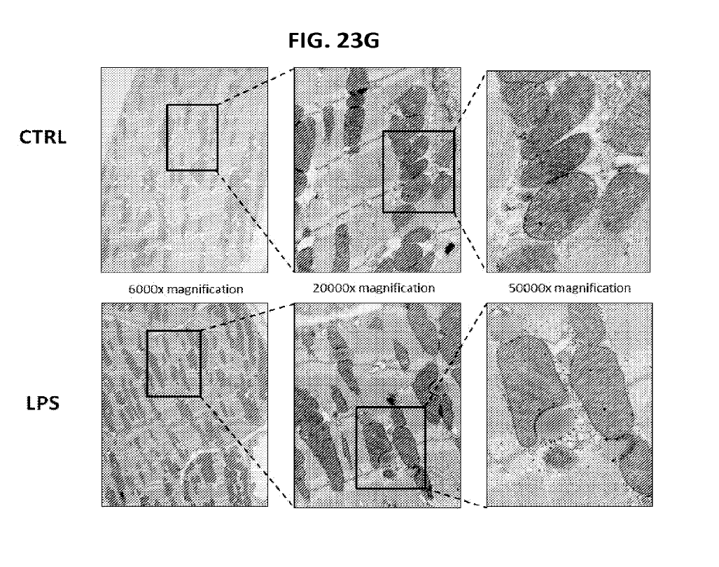

Our group²⁰⁰ and others²⁰¹⁻²⁰⁴ have shown that administration of LPS in mice reduces PPARα mRNA levels in the heart. The downregulation of PPARα may account for the LPS-driven reduction of cardiac FAO. The suppressing effects of LPS administration were confirmed on cardiac PPARα mRNA levels (FIG. 23A) that was reduced by 78% and on heart function (FIGS. 23(B), 23(C)), as shown by 2D-echocardiography that indicated 50% reduction in fractional shortening. Compromised cardiac function was associated with reduced cardiac FAO (FIG. 23(D)) and ATP (FIG. 23(E)) levels. Despite the energy deficiency state of the heart phosphorylation of AMP kinase (AMPK) was not stimulated (FIG. 23(F)). Mitochondrial structure analysis and intracellular arrangement by electron microscopy (at 6000×, 20,000×, and 50,000× magnification) of cardiac tissue from LPS-treated C57BL/6 mice showed that LPS treatment did not affect either the morphology of mitochondria or their intracellular arrangement as compared to control mice that were treated with saline. (FIG. 23(G))

### Example 8

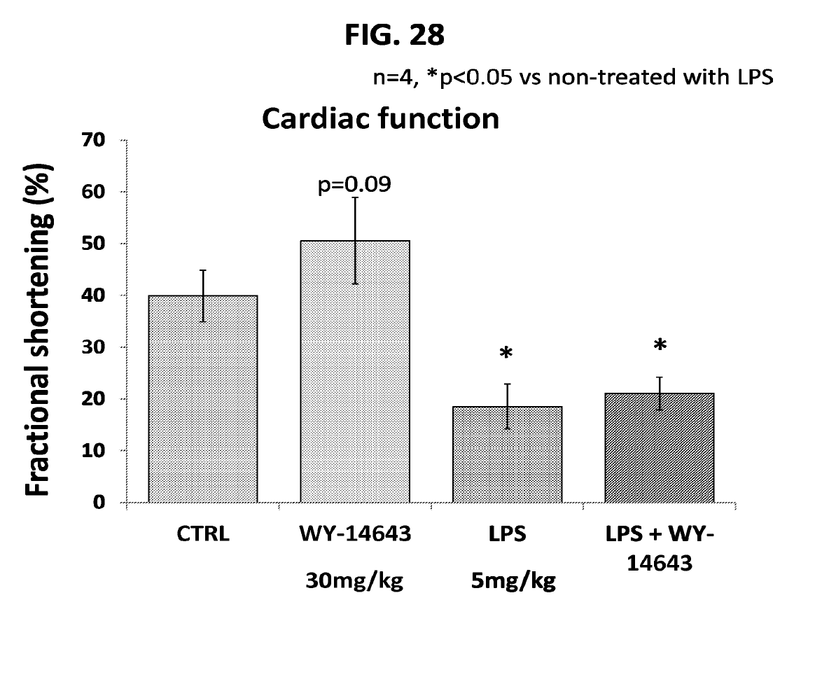

C57BL/6 mice were treated with combination of LPS (5 mg/kg) and the pharmacological PPARα agonist (WY-14643, 30 mg/kg). Administration of the PPARα agonist generated a trend for improved fractional shortening by 25% (p=0.09) in control saline-treated mice. FIG. 28.

### Example 9

A previous study of our group showed that cardiac FAO and energy production are increased in Ppara^{-/-} mice that express PPARγ in cardiomyocytes (αMHC-PPARγxPPARα^{-/-}) ²⁰⁵In order to assess whether PPARγ may substitute for the reduced PPARα and rescue FAO, 5 mg/kg LPS was administered to αMHC-PPARγ mice.²⁰⁶ Treatment of αMHC-PPARγ mice with LPS activated JNK (FIG. 24A) and downregulated PPARα mRNA levels by 78%. FIG. 24(B). Opposite to the inhibitory effect of LPS in C57BL/6 mice mRNA levels of genes mediating FAO were increased in the hearts of LPS-treated αMHC-PPARγ mice. Specifically, the mRNA levels of AOX, PPARγ, PGC-1α and Cpt-1β were increased by 27.1-, 31.6-, 30.1- and 14.3-fold, respectively, as compared to αMHC-PPARγ mice that were treated with saline. FIG. 24(B). On the other hand cardiac PGC-1β and CD36 gene expression levels were reduced by 76% and 93%, respectively. FIG. 24(B). Perilipin 5 mRNA levels were also reduced by 50%, while estrogen-related receptor (ERR)α and perilipin 2 were not modulated by treatment of the αMHC-PPARγ mice with LPS. FIG. 24(B). PDK4 mRNA levels were increased by 5.4-fold, FIG. 24(B), indicating that LPS-treated αMHC-PPARγ mice were unlikely to have switched to greater glucose utilization. Consistent with cardiac FAO-associated gene expression profile, the levels of FAO were increased by 2.6-fold in LPS-treated αMHC-PPARγ mice as compared to saline treated αMHC-PPARγ mice. FIG. 24(C). Accordingly, cardiac ATP levels of LPS-treated αMHC-PPARγ mice were also increased by 2.2-fold. FIG. 24(D). Phosphorylated AMPK levels were reduced in the LPS-treated mice that express PPARγ in cardiomyocytes and have increased cardiac energy production. FIG. 24(A). Therefore the αMHC-PPARγ transgenic mice were protected from LPS-induced defective FAO and reduction in ATP levels.

The improvement of cardiac FAO was associated with prevention of LPS-mediated cardiac dysfunction but not the induction of inflammation. 2D-echocardiography (FIG. 24(E)) showed preserved fractional shortening levels in LPS-treated αMHC-PPARγ mice. FIG. 24(F). Inflammation is a major component of sepsis and its alleviation has been proposed as a therapeutic approach in sepsis based on studies in animal models that are knock-out for inflammation-related genes.²⁰⁷⁻²¹⁰These mice are resistant to LPS-mediated sepsis and have improved cardiac function or viability. However, PPARγ-mediated prevention of cardiac dysfunction in LPS-treated mice was not accompanied by alleviation of inflammation, as shown by cardiac mRNA levels of IL-1α (FIG. 24(G)); IL-6 (FIG. 24(H)); and TNFα (FIG. 24(I) that were elevated by, 23-, 285- and 12.5-fold, respectively. Thus, the beneficial effect of PPARγ in the prevention of cardiac dysfunction during sepsis cannot be accounted for by anti-inflammatory effect.

### Example 10

Prevention

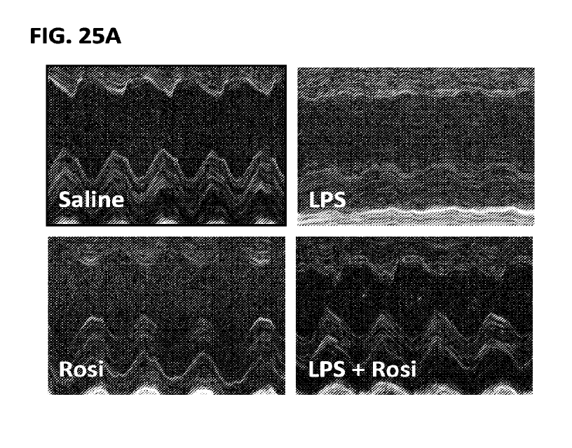

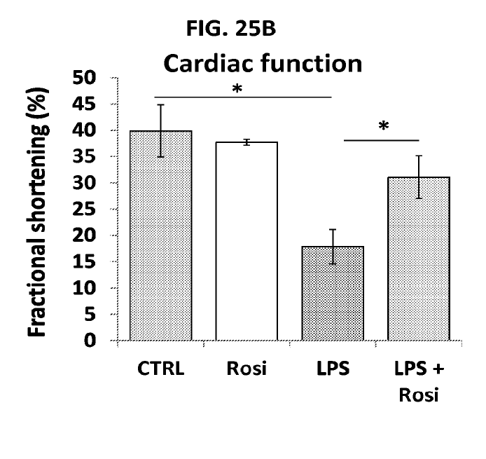

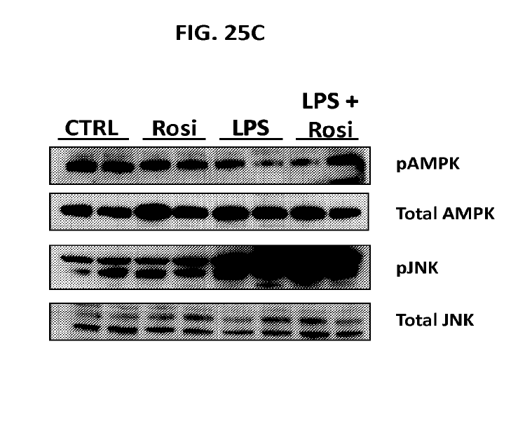

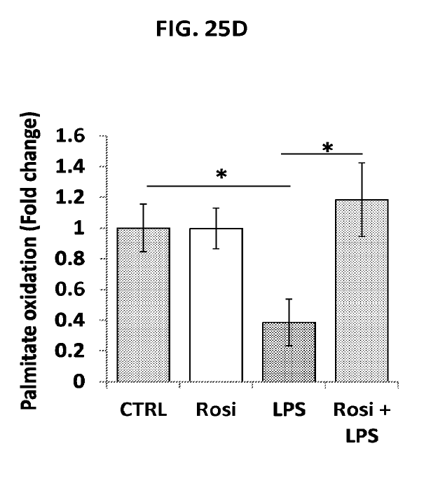

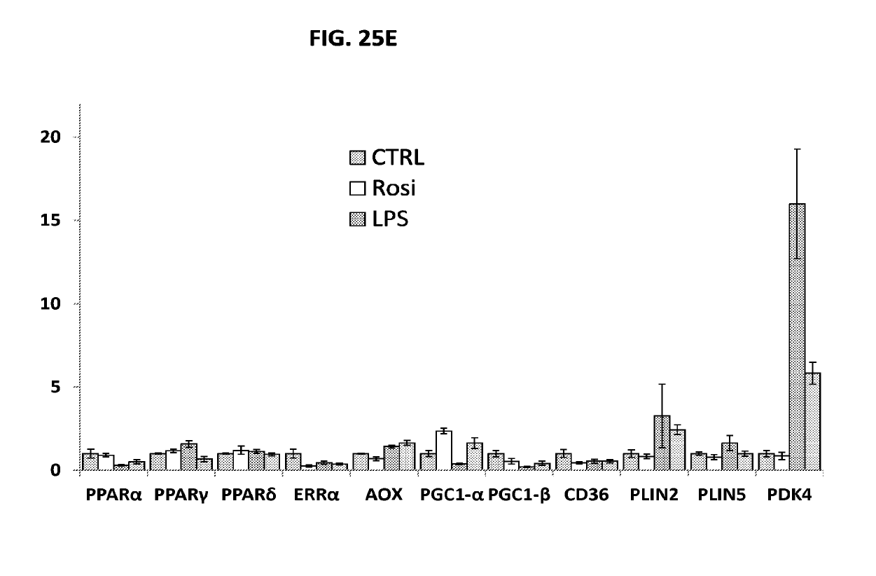

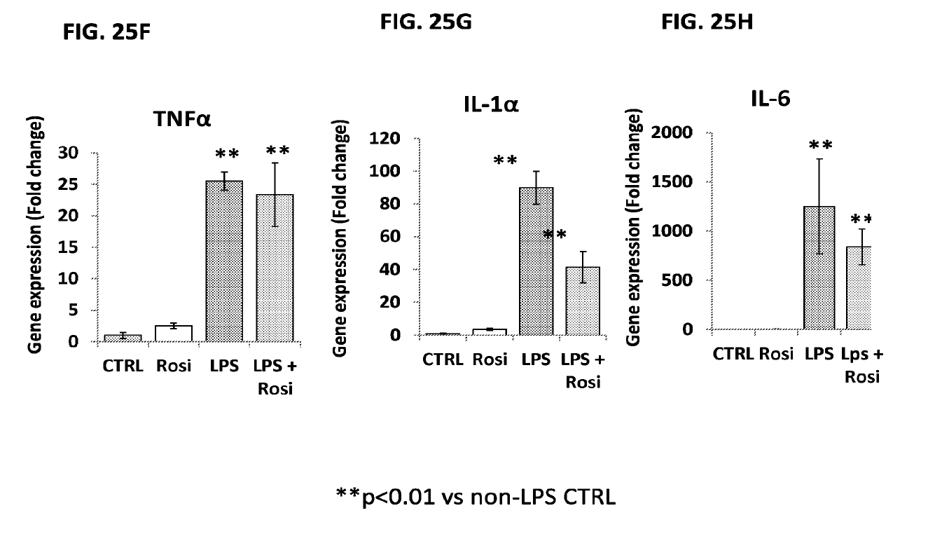

In order to assess whether pharmacologic activation of PPARγ prevents LPS-mediated cardiac dysfunction, C57BL/6 mice were treated parenterally with the PPARγ agonist, rosiglitazone (35 mg/kg) 30 min prior to the administration of LPS. Administration of rosiglitazone in LPS-treated mice prevented LPS-mediated cardiac dysfunction, and did not reduce left ventricular fractional shortening. FIGS. 25(A), 25(B). LPS treatment of mice induced phosphorylation of JNK and reduced phospho-AMPK in both controls and rosiglitazone-treated animals. FIG. 25(C). Despite the increased levels of cardiac pJNK, cardiac FAO was not reduced in mice receiving both LPS and rosiglitazone. FIG. 25(D). The improvement in FAO of LPS-treated mice that were co-administered with rosiglitazone was associated with increased gene expression levels of PGC1-α (4-fold) and PGC-1β (2.1-fold) levels, as well as with reduced perilipin 5 (60%) mRNA levels (FIG. 25(E), as compared to mice that were treated with LPS only. On the other hand, the LPS-mediated reduction of PPARα, ERRα and CD36 mRNA levels was not prevented by rosiglitazone treatment. FIG. 25(E). In addition, cardiac perilipin 2 and AOX mRNA was increased by 3.3- and 42%, respectively in LPS-treated mice and remained elevated in mice treated with LPS and rosiglitazone. FIG. 25(E). PPARγ mRNA was increased by 57% following LPS treatment and was normalized following administration of rosiglitazone in LPS-treated animals, while PPARδ was not modulated in any of the treatment groups. FIG. 25(E). PDK4 was increased 16-fold by LPS treatment and was reduced but remained significantly higher (5.8-fold) than saline-treated mice in mice that received LPS and rosiglitazone treatment. FIG. 25(E).

### Example 11

In order to test whether rosiglitazone-mediated improvement of cardiac function in LPS-treated C57BL/6 mice was due to attenuation of inflammation cardiac gene expression levels of TNFα, IL-1α and IL-6 were assessed. Analysis of cardiac mRNA with qRT-PCR showed that TNFα, IL-1α and IL-6 levels were increased by 26-, 90- and 1250-fold, respectively in LPS-treated mice. FIGS. 25(F)-25(H). Combined treatment with LPS and rosiglitazone did not prevent the increase of inflammatory markers (FIGS. 25(F)-25(H)), thus indicating that the improvement in cardiac function by rosiglitazone was not associated with attenuation of LPS-triggered inflammation.

### Example 12

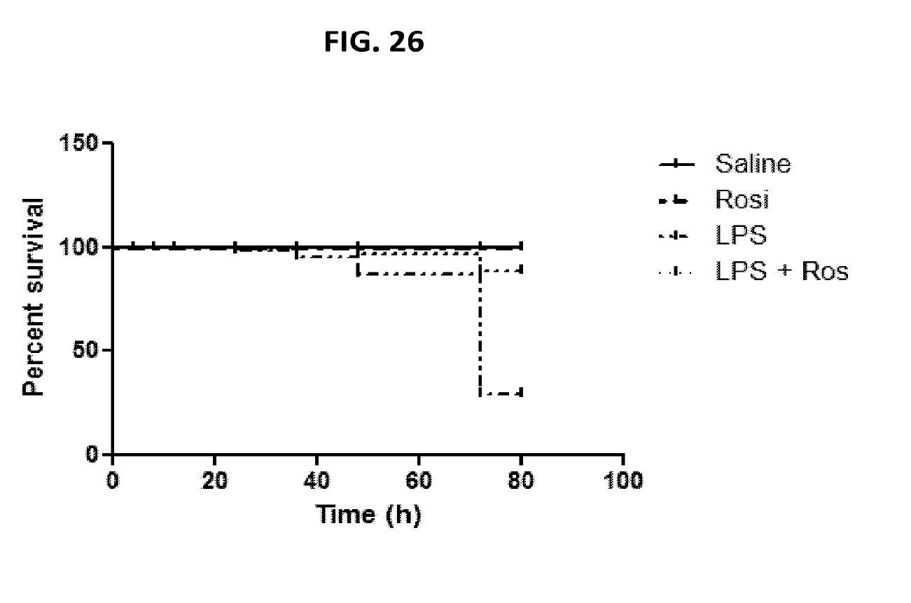

In order to assess whether rosiglitazone may improve survival during LPS-induced sepsis, C57BL/6 mice were administered with a lethal dose of LPS (15 mg/kg, i.p.) or combination of LPS and daily injections of rosiglitazone (35 mg/kg). Mice that were treated with LPS alone started dying 36 hours post-injection with the vast majority of lethal events occurring between 48 hours and 72 hours post-injection. Table 1. I.p administration of rosiglitazone on daily basis reduced significantly the lethal events of the LPS-treated mice (FIG. 26).

TABLE 1

| | | | No of | Survivors | Survivors | Survivors | Survivors | Survivors |
|---|---|---|---|---|---|---|---|---|
| Strain | Gender | Treatment | mice | 24 h | 36 h | 48 h | 60 h | 72 h |
| C57BL/6 | F | Saline + DMSO | 8 | 8 | 8 | 8 | 8 | 8 |
| C57BL/6 | M | Saline + | 8 | 8 | 8 | 8 | 8 | 8 |
| | | Rosiglitazone | | | | | | |
| C57BL/6 | F | LPS + DMSO | 15 | 14 | 14 | 10 | 5 | 2 |
| C57BL/6 | M | LPS + | 17 | 17 | 16 | 16 | 15 | 13 |
| | | Rosiglitazone | | | | | | |

LPS: 15 mg/kg
Rosiglitazone: 33 mg/kg/day

### REFERENCES

1. Annane D, Bellissant E, Cavaillon J M. Septic shock. Lancet. 2005; 365:63-78
2. Levy M M, Fink M P, Marshall J C, Abraham E, Angus D, Cook D, Cohen J, Opal S M, Vincent J L, Ramsay G. 2001 sccm/esicm/accp/ats/sis international sepsis definitions conference. Crit Care Med. 2003; 31:1250-1256
3. Akira S, Takeda K, Kaisho T. Toll-like receptors: Critical proteins linking innate and acquired immunity. Nat Immunol. 2001; 2:675-680
4. Ren J, Wu S. A burning issue: Do sepsis and systemic inflammatory response syndrome (sirs) directly contribute to cardiac dysfunction? Front Biosci. 2006; 11:15-22
5. Hunter J D, Doddi M. Sepsis and the heart. Br J Anaesth. 2010; 104:3-11
6. Kumar A, Thota V, Dee L, Olson J, Uretz E, Parrillo J E. Tumor necrosis factor alpha and interleukin 1beta are responsible for in vitro myocardial cell depression induced by human septic shock serum. J Exp Med. 1996; 183:949-958
7. Hoffmann J N, Werdan K, Hartl W H, Jochum M, Faist E, Inthorn D. Hemofiltrate from patients with severe sepsis and depressed left ventricular contractility contains cardiotoxic compounds. Shock. 1999; 12:174-180
8. Natanson C, Eichenholz P W, Danner R L, Eichacker P Q, Hoffman W D, Kuo G C, Banks S M, MacVittie T J, Parrillo J E. Endotoxin and tumor necrosis factor challenges in dogs simulate the cardiovascular profile of human septic shock. J Exp Med. 1989; 169:823-832
9. Finkel M S, Oddis C V, Jacob T D, Watkins S C, Hattler B G, Simmons R L. Negative inotropic effects of cytokines on the heart mediated by nitric oxide. Science. 1992; 257:387-389
10. Stein B, Frank P, Schmitz W, Scholz H, Thoenes M. Endotoxin and cytokines induce direct cardiodepressive effects in mammalian cardiomyocytes via induction of nitric oxide synthase. J Mol Cell Cardiol. 1996; 28:1631-1639
11. Schulz R, Panas D L, Catena R, Moncada S, Olley P M, Lopaschuk G D. The role of nitric oxide in cardiac depression induced by interleukin-1 beta and tumour necrosis factor-alpha. Br J Pharmacol. 1995; 114:27-34
12. Zhong J, Hwang T C, Adams H R, Rubin L J. Reduced 1-type calcium current in ventricular myocytes from endotoxemic guinea pigs. Am J Physiol. 1997; 273:H2312-2324
13. Goldhaber J I, Kim K H, Natterson P D, Lawrence T, Yang P, Weiss J N. Effects of tnf-alpha on [ca2+]i and contractility in isolated adult rabbit ventricular myocytes. Am J Physiol. 1996; 271:H1449-1455
14. Zuckerman S H, Evans G F, Guthrie L. Transcriptional and post-transcriptional mechanisms involved in the differential expression of 1 ps-induced il-1 and tnf mrna. Immunology. 1991; 73:460-465
15. Hambleton J, Weinstein S L, Lem L, DeFranco A L. Activation of c-jun n-terminal kinase in bacterial lipopolysaccharide-stimulated macrophages. Proc Natl Acad Sci USA. 1996; 93:2774-2778
16. Sanghera J S, Weinstein S L, Aluwalia M, Girn J, Pelech S L. Activation of multiple proline-directed kinases by bacterial lipopolysaccharide in murine macrophages. J Immunol. 1996; 156:4457-4465
17. Bone R C, Fisher C J, Jr., Clemmer T P, Slotman G J, Metz C A, Balk R A. A controlled clinical trial of high-dose methylprednisolone in the treatment of severe sepsis and septic shock. N Engl J Med. 1987; 317:653-658
18. Luce J M, Montgomery A B, Marks J D, Turner J, Metz C A, Murray J F. Ineffectiveness of high-dose methylprednisolone in preventing parenchymal lung injury and improving mortality in patients with septic shock. Am Rev Respir Dis. 1988; 138:62-68
19. Annane D, Bellissant E, Bollaert P E, Briegel J, Confalonieri M, De Gaudio R, Keh D, Kupfer Y, Oppert M, Meduri G U. Corticosteroids in the treatment of severe sepsis and septic shock in adults: A systematic review. JAMA. 2009; 301:2362-2375
20. Fisher C J, Jr., Dhainaut J F, Opal S M, Pribble J P, Balk R A, Slotman G J, Iberti T J, Rackow E C, Shapiro M J, Greenman R L, et al. Recombinant human interleukin 1 receptor antagonist in the treatment of patients with sepsis syndrome. Results from a randomized, double-blind, placebo-controlled trial. Phase iii rhil-1ra sepsis syndrome study group. JAMA. 1994; 271:1836-1843
21. Opal S M, Fisher C J, Jr., Dhainaut J F, Vincent J L, Brase R, Lowry S F, Sadoff J C, Slotman G J, Levy H, Balk R A, Shelly M P, Pribble J P, LaBrecque J F, Lookabaugh J, Donovan H, Dubin H, Baughman R, Norman J, DeMaria E, Matzel K, Abraham E, Seneff M. Confirmatory interleukin-1 receptor antagonist trial in severe sepsis: A phase iii, randomized, double-blind, placebo-controlled, multicenter trial. The interleukin-1 receptor antagonist sepsis investigator group. Crit Care Med. 1997; 25:1115-1124
22. Reinhart K, Karzai W. Anti-tumor necrosis factor therapy in sepsis: Update on clinical trials and lessons learned. Crit Care Med. 2001; 29:S121-125
23. Spitzer J J, Bechtel A A, Archer L T, Black M R, Hinshaw L B. Myocardial substrate utilization in dogs following endotoxin administration. Am J Physiol. 1974; 227:132-136
24. Lanza-Jacoby S, Feagans K, Tabares A. Fatty acid metabolism in the heart during escherichia coli sepsis in the rat. Circ Shock. 1989; 29:361-370
25. Liu M S, Spitzer J J. In vitro effects of e. Coli endotoxin on fatty acid and lactate oxidation in canine myocardium. Circ Shock. 1977; 4:181-190
26. Bagby G J, Spitzer J A. Lipoprotein lipase activity in rat heart and adipose tissue during endotoxic shock. Am J Physiol. 1980; 238:H325-330
27. Feingold K R, Marshall M, Gulli R, Moser A H, Grunfeld C. Effect of endotoxin and cytokines on lipoprotein lipase activity in mice. Arterioscler Thromb. 1994; 14:1866-1872
28. Feingold K, Kim M S, Shigenaga J, Moser A, Grunfeld C. Altered expression of nuclear hormone receptors and coactivators in mouse heart during the acute-phase response. Am J Physiol Endocrinol Metab. 2004; 286:E201-207
29. Jia L, Takahashi M, Morimoto H, Takahashi S, Izawa A, Ise H, Iwasaki T, Hattori H, Wu K J, Ikeda U. Changes in cardiac lipid metabolism during sepsis: The essential role of very low-density lipoprotein receptors. Cardiovasc Res. 2006; 69:545-555
30. Memon R A, Bass N M, Moser A H, Fuller J, Appel R, Grunfeld C, Feingold K R. Down-regulation of liver and heart specific fatty acid binding proteins by endotoxin and cytokines in vivo. Biochim Biophys Acta. 1999; 1440:118-126
31. Memon R A, Fuller J, Moser A H, Smith P J, Feingold K R, Grunfeld C. In vivo regulation of acyl-coa synthetase mrna and activity by endotoxin and cytokines. Am J Physiol. 1998; 275:E64-72
32. Ballard F B, Danforth W H, Naegle S, Bing R J. Myocardial metabolism of fatty acids. J Clin Invest. 1960; 39:717-723
33. Neubauer S. The failing heart—an engine out of fuel. N Engl J Med. 2007; 356:1140-1151
34. Osorio J C, Stanley W C, Linke A, Castellari M, Diep Q N, Panchal A R, Hintze T H, Lopaschuk G D, Recchia F A. Impaired myocardial fatty acid oxidation and reduced protein expression of retinoid×receptor-alpha in pacing-induced heart failure. Circulation. 2002; 106:606-612
35. Tessier J P, Thurner B, Jungling E, Luckhoff A, Fischer Y. Impairment of glucose metabolism in hearts from rats treated with endotoxin. Cardiovasc Res. 2003; 60:119-130
36. Finck B N, Lehman J J, Leone T C, Welch M J, Bennett M J, Kovacs A, Han X, Gross R W, Kozak R, Lopaschuk G D, Kelly D P. The cardiac phenotype induced by pparalpha overexpression mimics that caused by diabetes mellitus. J Clin Invest. 2002; 109:121-130
37. Finck B N, Bernal-Mizrachi C, Han D H, Coleman T, Sambandam N, LaRiviere L L, Holloszy J O, Semenkovich C F, Kelly D P. A potential link between muscle peroxisome proliferator-activated receptor-alpha signaling and obesity-related diabetes. Cell Metab. 2005; 1:133-144
38. Cheng L, Ding G, Qin Q, Xiao Y, Woods D, Chen Y E, Yang Q. Peroxisome proliferator-activated receptor delta activates fatty acid oxidation in cultured neonatal and adult cardiomyocytes. Biochem Biophys Res Commun. 2004; 313:277-286
39. Tontonoz P, Hu E, Spiegelman B M. Stimulation of adipogenesis in fibroblasts by ppar gamma 2, a lipid-activated transcription factor. Cell. 1994; 79:1147-1156
40. Son N H, Park T S, Yamashita H, Yokoyama M, Huggins L A, Okajima K, Homma S, Szabolcs M J, Huang L S, Goldberg I J. Cardiomyocyte expression of ppargamma leads to cardiac dysfunction in mice. J Clin Invest. 2007; 117:2791-2801
41. Son N H, Yu S, Tuinei J, Arai K, Hamai H, Homma S, Shulman G I, Abel E D, Goldberg U. Ppargamma-induced cardiolipotoxicity in mice is ameliorated by pparalpha deficiency despite increases in fatty acid oxidation. J Clin Invest. 2010; 120:3443-3454
42. Cha B S, Ciaraldi T P, Park K S, Carter L, Mudaliar S R, Henry R R. Impaired fatty acid metabolism in type 2 diabetic skeletal muscle cells is reversed by ppargamma agonists. Am J Physiol Endocrinol Metab. 2005; 289:E151-159
43. Maitra U, Chang S, Singh N, Li L. Molecular mechanism underlying the suppression of lipid oxidation during endotoxemia. Mol Immunol. 2009; 47:420-425
44. Vega R B, Huss J M, Kelly D P. The coactivator pgc-1 cooperates with peroxisome proliferator-activated receptor alpha in transcriptional control of nuclear genes encoding mitochondrial fatty acid oxidation enzymes. Mol Cell Biol. 2000; 20:1868-1876
45. Lemberger T, Staels B, Saladin R, Desvergne B, Auwerx J, Wahli W. Regulation of the peroxisome proliferator-activated receptor alpha gene by glucocorticoids. J Biol Chem. 1994; 269:24527-24530
46. Pineda Torra I, Claudel T, Duval C, Kosykh V, Fruchart J C, Staels B. Bile acids induce the expression of the human peroxisome proliferator-activated receptor alpha gene via activation of the farnesoid×receptor. Mol Endocrinol. 2003; 17:259-272
47. Lee W J, Kim M, Park H S, Kim H S, Jeon M J, Oh K S, Koh E H, Won J C, Kim M S, Oh G T, Yoon M, Lee K U, Park J Y. Ampk activation increases fatty acid oxidation in skeletal muscle by activating pparalpha and pgc-1. Biochem Biophys Res Commun. 2006; 340:291-295
48. Meng R S, Pei Z H, Yin R, Zhang C X, Chen B L, Zhang Y, Liu D, Xu A L, Dong Y G. Adenosine monophosphate-activated protein kinase inhibits cardiac hypertrophy through reactivating peroxisome proliferator-activated receptor-alpha signaling pathway. Eur J Pharmacol. 2009; 620:63-70
49. Ravnskjaer K, Boergesen M, Dalgaard L T, Mandrup S. Glucose-induced repression of pparalpha gene expression in pancreatic beta-cells involves pp 2a activation and ampk inactivation. J Mol Endocrinol. 2006; 36:289-299
50. Huss J M, Torra I P, Staels B, Giguere V, Kelly D P. Estrogen-related receptor alpha directs peroxisome proliferator-activated receptor alpha signaling in the transcriptional control of energy metabolism in cardiac and skeletal muscle. Mol Cell Biol. 2004; 24:9079-9091
51. Valmaseda A, Carmona M C, Barbera M J, Vinas O, Mampel T, Iglesias R, Villarroya F, Giralt M. Opposite regulation of ppar-alpha and -gamma gene expression by both their ligands and retinoic acid in brown adipocytes. Mol Cell Endocrinol. 1999; 154:101-109
52. Beigneux A P, Moser A H, Shigenaga J K, Grunfeld C, Feingold K R. The acute phase response is associated with retinoid×receptor repression in rodent liver. J Biol Chem. 2000; 275:16390-16399
53. Yaacob N S, Norazmi M N, Gibson G G, Kass G E. The transcription of the peroxisome proliferator-activated receptor alpha gene is regulated by protein kinase c. Toxicol Lett. 2001; 125:133-141
54. Iemitsu M, Miyauchi T, Maeda S, Tanabe T, Takanashi M, Irukayama-Tomobe Y, Sakai S, Ohmori H, Matsuda M, Yamaguchi I. Aging-induced decrease in the ppar-alpha level in hearts is improved by exercise training. Am J Physiol Heart Circ Physiol. 2002; 283:H1750-1760
55. Vallanat B, Anderson S P, Brown-Borg H M, Ren H, Kersten S, Jonnalagadda S, Srinivasan R, Corton J C. Analysis of the heat shock response in mouse liver reveals transcriptional dependence on the nuclear receptor peroxisome proliferator-activated receptor alpha (pparalpha). BMC Genomics. 2010; 11:16
56. Karbowska J, Kochan Z, Smolenski R T. Peroxisome proliferator-activated receptor alpha is downregulated in the failing human heart. Cell Mol Biol Lett. 2003; 8:49-53
57. Masamura K, Tanaka N, Yoshida M, Kato M, Kawai Y, Oida K, Miyamori I. Myocardial metabolic regulation through peroxisome proliferator-activated receptor alpha after myocardial infarction. Exp Clin Cardiol. 2003; 8:61-66
58. Narravula S, Colgan S P. Hypoxia-inducible factor 1-mediated inhibition of peroxisome proliferator-activated receptor alpha expression during hypoxia. J Immunol. 2001; 166:7543-7548
59. Razeghi P, Young M E, Abbasi S, Taegtmeyer H. Hypoxia in vivo decreases peroxisome proliferator-activated receptor alpha-regulated gene expression in rat heart. Biochem Biophys Res Commun. 2001; 287:5-10
60. Parmentier J H, Schohn H, Bronner M, Ferrari L, Batt A M, Dauca M, Kremers P. Regulation of cyp4a1 and peroxisome proliferator-activated receptor alpha expression by interleukin-1beta, interleukin-6, and dexamethasone in cultured fetal rat hepatocytes. Biochem Pharmacol. 1997; 54:889-898
61. Chu R, Lin Y, Rao M S, Reddy J K. Cloning and identification of rat deoxyuridine triphosphatase as an inhibitor of peroxisome proliferator-activated receptor alpha. J Biol Chem. 1996; 271:27670-27676
62. Shi Y, Hon M, Evans R M. The peroxisome proliferator-activated receptor delta, an integrator of transcriptional repression and nuclear receptor signaling. Proc Natl Acad Sci USA. 2002; 99:2613-2618
63. Cabrero A, Alegret M, Sanchez R M, Adzet T, Laguna J C, Carrera M V. Increased reactive oxygen species production down-regulates peroxisome proliferator-activated alpha pathway in c2c12 skeletal muscle cells. J Biol Chem. 2002; 277:10100-10107
64. Roduit R, Morin J, Masse F, Segall L, Roche E, Newgard C B, Assimacopoulos-Jeannet F, Prentki M. Glucose down-regulates the expression of the peroxisome proliferator-activated receptor-alpha gene in the pancreatic beta-cell. J Biol Chem. 2000; 275:35799-35806
65. Joly E, Roduit R, Peyot M L, Habinowski S A, Ruderman N B, Witters L A, Prentki M. Glucose represses pparalpha gene expression via amp-activated protein kinase but not via p38 mitogen-activated protein kinase in the pancreatic beta-cell. J Diabetes. 2009; 1:263-272
66. Panadero M, Vidal H, Herrera E, Bocos C. Nutritionally induced changes in the peroxisome proliferator-activated receptor-alpha gene expression in liver of suckling rats are dependent on insulinaemia. Arch Biochem Biophys. 2001; 394:182-188
67. Cook S A, Matsui T, Li L, Rosenzweig A. Transcriptional effects of chronic akt activation in the heart. J Biol Chem. 2002; 277:22528-22533
68. Riu E, Ferre T, Mas A, Hidalgo A, Franckhauser S, Bosch F. Overexpression of c-myc in diabetic mice restores altered expression of the transcription factor genes that regulate liver metabolism. Biochem J. 2002; 368:931-937
69. Zhou Y C, Waxman D J. Stat5b down-regulates peroxisome proliferator-activated receptor alpha transcription by inhibition of ligand-independent activation function region-1 trans-activation domain. J Biol Chem. 1999; 274:29874-29882
70. Carlsson L, Linden D, Jalouli M, Oscarsson J. Effects of fatty acids and growth hormone on liver fatty acid binding protein and pparalpha in rat liver. Am J Physiol Endocrinol Metab. 2001; 281:E772-781
71. Collett G P, Betts A M, Johnson M I, Pulimood A B, Cook S, Neal D E, Robson C N. Peroxisome proliferator-activated receptor alpha is an androgen-responsive gene in human prostate and is highly expressed in prostatic adenocarcinoma. Clin Cancer Res. 2000; 6:3241-3248
72. Tham D M, Martin-McNulty B, Wang Y X, Wilson D W, Vergona R, Sullivan M E, Dole W, Rutledge J C. Angiotensin ii is associated with activation of nf-kappab-mediated genes and downregulation of ppars. Physiol Genomics. 2002; 11:21-30
73. Schreiber S N, Knutti D, Brogli K, Uhlmann T, Kralli A. The transcriptional coactivator pgc-1 regulates the expression and activity of the orphan nuclear receptor estrogen-related receptor alpha (erralpha). J Biol Chem. 2003; 278:9013-9018
74. Chabowski A, Momken I, Coort S L, Calles-Escandon J, Tandon N N, Glatz J F, Luiken J J, Bonen A. Prolonged ampk activation increases the expression of fatty acid transporters in cardiac myocytes and perfused hearts. Mol Cell Biochem. 2006; 288:201-212
75. Makinde A O, Gamble J, Lopaschuk G D. Upregulation of 5′-amp-activated protein kinase is responsible for the increase in myocardial fatty acid oxidation rates following birth in the newborn rabbit. Circ Res. 1997; 80:482-489
76. Zong H, Ren J M, Young L H, Pypaert M, Mu J, Birnbaum M J, Shulman G I. Amp kinase is required for mitochondrial biogenesis in skeletal muscle in response to chronic energy deprivation. Proc Natl Acad Sci USA. 2002; 99:15983-15987
77. Long Y C, Barnes B R, Mahlapuu M, Steiler T L, Martinsson S, Leng Y, Wallberg-Henriksson H, Andersson L, Zierath J R. Role of amp-activated protein kinase in the coordinated expression of genes controlling glucose and lipid metabolism in mouse white skeletal muscle. Diabetologia. 2005; 48:2354-2364
78. Barnes B R, Long Y C, Steiler T L, Leng Y, Galuska D, Wojtaszewski J F, Andersson L, Zierath J R. Changes in exercise-induced gene expression in 5′-amp-activated protein kinase gamma3-null and gamma3 r225q transgenic mice. Diabetes. 2005; 54:3484-3489
79. Garcia-Roves P M, Osler M E, Holmstrom M H, Zierath J R. Gain-of-function r225q mutation in amp-activated protein kinase gamma3 subunit increases mitochondrial biogenesis in glycolytic skeletal muscle. J Biol Chem. 2008; 283:35724-35734
80. Hu T, Foxworthy P, Siesky A, Ficorilli J V, Gao H, Li S, Christe M, Ryan T, Cao G, Eacho P, Michael M D, Michael L F. Hepatic peroxisomal fatty acid beta-oxidation is regulated by liver×receptor alpha. Endocrinology. 2005; 146:5380-5387
81. Chawla A, Boisvert W A, Lee C H, Laffitte B A, Barak Y, Joseph S B, Liao D, Nagy L, Edwards P A, Curtiss L K, Evans R M, Tontonoz P. A ppar gamma-lxr-abcal pathway in macrophages is involved in cholesterol efflux and atherogenesis. Mol Cell. 2001; 7:161-171
82. Oberkofler H, Schraml E, Krempler F, Patsch W. Potentiation of liver×receptor transcriptional activity by peroxisome-proliferator-activated receptor gamma co-activator 1 alpha. Biochem J. 2003; 371:89-96
83. Ide T, Shimano H, Yoshikawa T, Yahagi N, Amemiya-Kudo M, Matsuzaka T, Nakakuki M, Yatoh S, Iizuka Y, Tomita S, Ohashi K, Takahashi A, Sone H, Gotoda T, Osuga J, Ishibashi S, Yamada N. Cross-talk between peroxisome proliferator-activated receptor (ppar) alpha and liver×receptor (l×r) in nutritional regulation of fatty acid metabolism. Ii. L×rs suppress lipid degradation gene promoters through inhibition of ppar signaling. Mol Endocrinol. 2003; 17:1255-1267
84. Wu S, Yin R, Ernest R, Li Y, Zhelyabovska O, Luo J, Yang Y, Yang Q. Liver×receptors are negative regulators of cardiac hypertrophy via suppressing nf-kappab signalling. Cardiovasc Res. 2009; 84:119-126
85. Clarke S L, Robinson C E, Gimble J M. Caat/enhancer binding proteins directly modulate transcription from the peroxisome proliferator-activated receptor gamma 2 promoter. Biochem Biophys Res Commun. 1997; 240:99-103
86. Wu Z, Rosen E D, Brun R, Hauser S, Adelmant G, Troy A E, McKeon C, Darlington G J, Spiegelman B M. Cross-regulation of c/ebp alpha and ppar gamma controls the transcriptional pathway of adipogenesis and insulin sensitivity. Mol Cell. 1999; 3:151-158
87. Wang X, Kilgore M W. Signal cross-talk between estrogen receptor alpha and beta and the peroxisome proliferator-activated receptor gamma1 in mda-mb-231 and mcf-7 breast cancer cells. Mol Cell Endocrinol. 2002; 194:123-133
88. Prusty D, Park B H, Davis K E, Farmer S R. Activation of mek/erk signaling promotes adipogenesis by enhancing peroxisome proliferator-activated receptor gamma (ppargamma) and c/ebpalpha gene expression during the differentiation of 3t3-11 preadipocytes. J Biol Chem. 2002; 277:46226-46232
89. Xiao H, Leblanc S E, Wu Q, Konda S, Salma N, Marfella C G, Ohkawa Y, Imbalzano A N. Chromatin accessibility and transcription factor binding at the ppargamma2 promoter during adipogenesis is protein kinase a-dependent. J Cell Physiol. 2011; 226:86-93
90. Kintscher U, Wakino S, Bruemmer D, Goetze S, Graf K, Hsueh W A, Law R E. Tgf-beta(1) induces peroxisome proliferator-activated receptor gamma1 and gamma2 expression in human thp-1 monocytes. Biochem Biophys Res Commun. 2002; 297:794-799
91. Hata K, Nishimura R, Ikeda F, Yamashita K, Matsubara T, Nokubi T, Yoneda T. Differential roles of smad1 and p38 kinase in regulation of peroxisome proliferator-activating receptor gamma during bone morphogenetic protein 2-induced adipogenesis. Mol Biol Cell. 2003; 14:545-555
92. Fu M, Zhang J, Lin Y, Zhu X, Zhao L, Ahmad M, Ehrengruber M U, Chen Y E. Early stimulation and late inhibition of peroxisome proliferator-activated receptor gamma (ppar gamma) gene expression by transforming growth factor beta in human aortic smooth muscle cells: Role of early growth-response factor-1 (egr-1), activator protein 1 (ap1) and smads. Biochem J. 2003; 370:1019-1025
93. Chambrier C, Bastard J P, Rieusset J, Chevillotte E, Bonnefont-Rousselot D, Therond P, Hainque B, Riou J P, Laville M, Vidal H. Eicosapentaenoic acid induces mrna expression of peroxisome proliferator-activated receptor gamma. Obes Res. 2002; 10:518-525
94. Oster R T, Tishinsky J M, Yuan Z, Robinson L E. Docosahexaenoic acid increases cellular adiponectin mrna and secreted adiponectin protein, as well as ppargamma mma, in 3t3-11 adipocytes. Appl Physiol Nutr Metab. 2010; 35:783-789
95. Sundvold H, Lien S. Identification of a novel peroxisome proliferator-activated receptor (ppar) gamma promoter in man and transactivation by the nuclear receptor roralpha1. Biochem Biophys Res Commun. 2001; 287:383-390
96. Gupta R K, Arany Z, Seale P, Mepani R J, Ye L, Conroe H M, Roby Y A, Kulaga H, Reed R R, Spiegelman B M. Transcriptional control of preadipocyte determination by zfp423. Nature. 2010; 464:619-623
97. Landrier J F, Gouranton E, El Yazidi C, Malezet C, Balaguer P, Borel P, Amiot M J. Adiponectin expression is induced by vitamin e via a peroxisome proliferator-activated receptor gamma-dependent mechanism. Endocrinology. 2009; 150:5318-5325
98. Welch J S, Ricote M, Akiyama T E, Gonzalez F J, Glass C K. Ppargamma and ppardelta negatively regulate specific subsets of lipopolysaccharide and ifn-gamma target genes in macrophages. Proc Natl Acad Sci USA. 2003; 100:6712-6717
99. Jennewein C, von Knethen A, Schmid T, Brune B. Microrna-27b contributes to lipopolysaccharide-mediated peroxisome proliferator-activated receptor gamma (ppargamma) mrna destabilization. J Biol Chem. 2010; 285:11846-11853
100. Lee J, Jung E, Kim Y S, Roh K, Jung K H, Park D. Ultraviolet a regulates adipogenic differentiation of human adipose tissue-derived mesenchymal stem cells via up-regulation of kruppel-like factor 2. J Biol Chem. 2010; 285:32647-32656
101. Yang Q, Chen C, Wu S, Zhang Y, Mao X, Wang W. Advanced glycation end products downregulates peroxisome proliferator-activated receptor gamma expression in cultured rabbit chondrocyte through mapk pathway. Eur J Pharmacol. 2010; 649:108-114
102. Lee J, Jung E, Huh S, Kim Y S, Kim Y W, Park D. Anti-adipogenesis by 6-thioinosine is mediated by downregulation of ppar gamma through jnk-dependent upregulation of inos. Cell Mol Life Sci. 2010; 67:467-481
103. Zhang B, Berger J, Hu E, Szalkowski D, White-Carrington S, Spiegelman B M, Moller D E. Negative regulation of peroxisome proliferator-activated receptor-gamma gene expression contributes to the antiadipogenic effects of tumor necrosis factor-alpha. Mol Endocrinol. 1996; 10:1457-1466
104. Xing H, Northrop J P, Grove J R, Kilpatrick K E, Su J L, Ringold G M. Tnf alpha-mediated inhibition and reversal of adipocyte differentiation is accompanied by suppressed expression of ppargamma without effects on pref-1 expression. Endocrinology. 1997; 138:2776-2783
105. Meng L, Zhou J, Sasano H, Suzuki T, Zeitoun K M, Bulun S E. Tumor necrosis factor alpha and interleukin 11 secreted by malignant breast epithelial cells inhibit adipocyte differentiation by selectively down-regulating ccaat/enhancer binding protein alpha and peroxisome proliferator-activated receptor gamma: Mechanism of desmoplastic reaction. Cancer Res. 2001; 61:2250-2255
106. Kurebayashi S, Sumitani S, Kasayama S, Jetten A M, Hirose T. Tnf-alpha inhibits 3t3-11 adipocyte differentiation without downregulating the expression of c/ebpbeta and delta. Endocr J. 2001; 48:249-253
107. Park S H, Choi H J, Yang H, Do K H, Kim J, Moon Y. Repression of peroxisome proliferator-activated receptor gamma by mucosal ribotoxic insult-activated ccaat/enhancer-binding protein homologous protein. J Immunol. 2010; 185:5522-5530
108. Lobo G P, Amengual J, Li H N, Golczak M, Bonet M L, Palczewski K, von Lintig J. Beta,beta-carotene decreases peroxisome proliferator receptor gamma activity and reduces lipid storage capacity of adipocytes in a beta,beta-carotene oxygenase 1-dependent manner. J Biol Chem. 2010; 285:27891-27899
109. Xiao J, Wang N L, Sun B, Cai G P. Estrogen receptor mediates the effects of pseudoprotodiocsin on adipogenesis in 3t3-11 cells. Am J Physiol Cell Physiol. 2010; 299:C128-138
110. Waite K J, Floyd Z E, Arbour-Reily P, Stephens J M. Interferon-gamma-induced regulation of peroxisome proliferator-activated receptor gamma and stats in adipocytes. J Biol Chem. 2001; 276:7062-7068
111. Zhou Y, Jia X, Qin J, Lu C, Zhu H, Li X, Han X, Sun X. Leptin inhibits ppargamma gene expression in hepatic stellate cells in the mouse model of liver damage. Mol Cell Endocrinol. 2010; 323:193-200
112. Escher P, Braissant O, Basu-Modak S, Michalik L, Wahli W, Desvergne B. Rat ppars: Quantitative analysis in adult rat tissues and regulation in fasting and refeeding. Endocrinology. 2001; 142:4195-4202
113. Kajita K, Ishizuka T, Mune T, Miura A, Ishizawa M, Kanoh Y, Kawai Y, Natsume Y, Yasuda K. Dehydroepiandrosterone down-regulates the expression of peroxisome proliferator-activated receptor gamma in adipocytes. Endocrinology. 2003; 144:253-259
114. Oishi Y, Manabe I, To be K, Tsushima K, Shindo T, Fujiu K, Nishimura G, Maemura K, Yamauchi T, Kubota N, Suzuki R, Kitamura T, Akira S, Kadowaki T, Nagai R. Kruppel-like transcription factor klf5 is a key regulator of adipocyte differentiation. Cell Metab. 2005; 1:27-39
115. Mori T, Sakaue H, Iguchi H, Gomi H, Okada Y, Takashima Y, Nakamura K, Nakamura T, Yamauchi T, Kubota N, Kadowaki T, Matsuki Y, Ogawa W, Hiramatsu R, Kasuga M. Role of kruppel-like factor 15 (klf15) in transcriptional regulation of adipogenesis. J Biol Chem. 2005; 280:12867-12875
116. Banerjee S S, Feinberg M W, Watanabe M, Gray S, Haspel R L, Denkinger D J, Kawahara R, Hauner H, Jain M K. The kruppel-like factor klf2 inhibits peroxisome proliferator-activated receptor-gamma expression and adipogenesis. J Biol Chem. 2003; 278:2581-2584
117. Kawamura Y, Tanaka Y, Kawamori R, Maeda S. Overexpression of kruppel-like factor 7 regulates adipocytokine gene expressions in human adipocytes and inhibits glucose-induced insulin secretion in pancreatic beta-cell line. Mol Endocrinol. 2006; 20:844-856
118. Oishi Y, Manabe I, To be K, Ohsugi M, Kubota T, Fujiu K, Maemura K, Kubota N, Kadowaki T, Nagai R. Sumoylation of kruppel-like transcription factor 5 acts as a molecular switch in transcriptional programs of lipid metabolism involving ppar-delta. Nat Med. 2008; 14:656-666
119. Li D, Yea S, Li S, Chen Z, Narla G, Banck M, Laborda J, Tan S, Friedman J M, Friedman S L, Walsh M J. Kruppel-like factor-6 promotes preadipocyte differentiation through histone deacetylase 3-dependent repression of dlk1. J Biol Chem. 2005; 280:26941-26952
120. Qi W, Chen X, Holian J, Tan C Y, Kelly D J, Pollock C A. Transcription factors kruppel-like factor 6 and peroxisome proliferator-activated receptor-{gamma} mediate high glucose-induced thioredoxin-interacting protein. Am J Pathol. 2009; 175:1858-1867
121. Finck B N, Kelly D P. Pgc-1 coactivators: Inducible regulators of energy metabolism in health and disease. J Clin Invest. 2006; 116:615-622
122. Liang J, Yang Y, Zhu X, Wang X, Chen R. Down-expression of pgc-1alpha partially mediated by jnk/c-jun through binding to cre site during apoptotic procedure in cerebellar granule neurons. J Neurosci Res. 2010; 88:1918-1925
123. Yu X X, Murray S F, Watts L, Booten S L, Tokorcheck J, Monia B P, Bhanot S. Reduction of jnk1 expression with antisense oligonucleotide improves adiposity in obese mice. Am J Physiol Endocrinol Metab. 2008; 295:E436-445
124. Russell R R, 3rd, Li J, Coven D L, Pypaert M, Zechner C, Palmeri M, Giordano F J, Mu J, Birnbaum M J, Young L H. Amp-activated protein kinase mediates ischemic glucose uptake and prevents postischemic cardiac dysfunction, apoptosis, and injury. J Clin Invest. 2004; 114:495-503
125. Qi D, Hu X, Wu X, Merk M, Leng L, Bucala R, Young L H. Cardiac macrophage migration inhibitory factor inhibits jnk pathway activation and injury during ischemia/reperfusion. J Clin Invest. 2009; 119:3807-3816
126. Davidson, M. M., Nesti, C., Palenzuela, L., Walker, W. F., Hernandez, E., Protas, L., Hirano, M., and Isaac, N. D. 2005. Novel cell lines derived from adult human ventricular cardiomyocytes. J Mol Cell Cardiol 39:133-147
127. Tsuiki, H., Tnani, M., Okamoto, I., Kenyon, L. C., Emlet, D. R., Holgado-Madruga, M., Lanham, I. S., Joynes, C. J., Vo, K. T., and Wong, A. J. 2003. Constitutively active forms of c-Jun N H2-terminal kinase are expressed in primary glial tumors. Cancer Res 63:250-255
128. Drosatos, K., Sanoudou, D., Kypreos, K. E., Kardassis, D., and Zannis, V. I. 2007. A dominant negative form of the transcription factor c-Jun affects genes that have opposing effects on lipid homeostasis in mice. J Biol Chem 282:19556-19564
129. Takuma, S., Suchiro, K., Cardinale, C., Hozumi, T., Yano, H., Shimizu, J., Mullis-Jansson, S., Sciacca, R., Wang, J., Burkhoff, D., et al. 2001. Anesthetic inhibition in ischemic and nonischemic murine heart: comparison with conscious echocardiographic approach. Am J Physiol Heart Circ Physiol 280:H2364-2370
130. Wang, C. Y., Mazer, S. P., Minamoto, K., Takuma, S., Homma, S., Yellin, M., Chess, L., Fard, A., Kalled, S. L., Oz, M. C., et al. 2002. Suppression of murine cardiac allograft arteriopathy by long-term blockade of CD40-CD 154 interactions. Circulation 105:1609-1614
131. Bone R C, Balk R A, Cerra F B, Dellinger R P, Fein A M, Knaus W A, Schein R M, Sibbald W J. Definitions for sepsis and organ failure and guidelines for the use of innovative therapies in sepsis. The accp/sccm consensus conference committee. American college of chest physicians/society of critical care medicine. Chest. 1992; 101:1644-1655
132. Ren J, Wu S. A burning issue: Do sepsis and systemic inflammatory response syndrome (sirs) directly contribute to cardiac dysfunction? Front Biosci. 2006; 11:15-22
133. Annane D, Bellissant E, Cavaillon J M. Septic shock. Lancet. 2005; 365:63-78
134. Levy M M, Fink M P, Marshall J C, Abraham E, Angus D, Cook D, Cohen J, Opal S M, Vincent J L, Ramsay G. 2001 sccm/esicm/accp/ats/sis international sepsis definitions conference. Crit Care Med. 2003; 31:1250-1256
135. Hunter J D, Doddi M. Sepsis and the heart. Br J Anaesth. 2010; 104:3-11
136. Carlson D L, Willis M S, White D J, Horton J W, Giroir B P. Tumor necrosis factor-alpha-induced caspase activation mediates endotoxin-related cardiac dysfunction. Crit Care Med. 2005; 33:1021-1028
137. Fallach R, Shainberg A, Avlas 0, Fainblut M, Chepurko Y, Porat E, Hochhauser E. Cardiomyocyte toll-like receptor 4 is involved in heart dysfunction following septic shock or myocardial ischemia. J Mol Cell Cardiol. 2010; 48:1236-1244
138. Knuefermann P, Nemoto S, Misra A, Nozaki N, Defreitas G, Goyert S M, Carabello B A, Mann D L, Vallejo J G. Cd14-deficient mice are protected against lipopolysaccharide-induced cardiac inflammation and left ventricular dysfunction. Circulation. 2002; 106:2608-2615
139. Ohlsson K, Bjork P, Bergenfeldt M, Hageman R, Thompson R C. Interleukin-1 receptor antagonist reduces mortality from endotoxin shock. Nature. 1990; 348:550-552
140. Bone R C, Fisher C J, Jr., Clemmer T P, Slotman G J, Metz C A, Balk R A. A controlled clinical trial of high-dose methylprednisolone in the treatment of severe sepsis and septic shock. N Engl J Med. 1987; 317:653-658
141. Luce J M, Montgomery A B, Marks J D, Turner J, Metz C A, Murray J F. Ineffectiveness of high-dose methylprednisolone in preventing parenchymal lung injury and improving mortality in patients with septic shock. Am Rev Respir Dis. 1988; 138:62-68
142. Annane D, Bellissant E, Bollaert P E, Briegel J, Confalonieri M, De Gaudio R, Keh D, Kupfer Y, Oppert M, Meduri G U. Corticosteroids in the treatment of severe sepsis and septic shock in adults: A systematic review. JAMA. 2009; 301:2362-2375
143. Fisher C J, Jr., Dhainaut J F, Opal S M, Pribble J P, Balk R A, Slotman G J, Iberti T J, Rackow E C, Shapiro M J, Greenman R L, et al. Recombinant human interleukin 1 receptor antagonist in the treatment of patients with sepsis syndrome. Results from a randomized, double-blind, placebo-controlled trial. Phase iii rhil-1ra sepsis syndrome study group. JAMA. 1994; 271:1836-1843
144. Opal S M, Fisher C J, Jr., Dhainaut J F, Vincent J L, Brase R, Lowry S F, Sadoff J C, Slotman G J, Levy H, Balk R A, Shelly M P, Pribble J P, LaBrecque J F, Lookabaugh J, Donovan H, Dubin H, Baughman R, Norman J, DeMaria E, Matzel K, Abraham E, Seneff M. Confirmatory interleukin-1 receptor antagonist trial in severe sepsis: A phase iii, randomized, double-blind, placebo-controlled, multicenter trial. The interleukin-1 receptor antagonist sepsis investigator group. Crit Care Med. 1997; 25:1115-1124
145. Reinhart K, Karzai W. Anti-tumor necrosis factor therapy in sepsis: Update on clinical trials and lessons learned. Crit Care Med. 2001; 29:S121-125
146. Feingold K R, Moser A, Patzek S M, Shigenaga J K, Grunfeld C. Infection decreases fatty acid oxidation and nuclear hormone receptors in the diaphragm. J Lipid Res. 2009; 50:2055-2063
147. Feingold K R, Wang Y, Moser A, Shigenaga J K, Grunfeld C. Lps decreases fatty acid oxidation and nuclear hormone receptors in the kidney. J Lipid Res. 2008; 49:2179-2187
148. Maitra U, Chang S, Singh N, Li L. Molecular mechanism underlying the suppression of lipid oxidation during endotoxemia. Mol Immunol. 2009; 47:420-425
149. van Rooij E, Marshall W S, Olson E N. Toward microrna-based therapeutics for heart disease: The sense in antisense. Circ Res. 2008; 103:919-928
150. van Rooij E, Sutherland L B, Liu N, Williams A H, McAnally J, Gerard R D, Richardson J A, Olson E N. A signature pattern of stress-responsive micrornas that can evoke cardiac hypertrophy and heart failure. Proc Natl Acad Sci USA. 2006; 103:18255-18260
151. van Rooij E, Sutherland L B, Qi X, Richardson J A, Hill J, Olson E N. Control of stress-dependent cardiac growth and gene expression by a microrna. Science. 2007; 316:575-579
152. Hambleton J, Weinstein S L, Lem L, DeFranco A L. Activation of c-jun n-terminal kinase in bacterial lipopolysaccharide-stimulated macrophages. Proc Natl Acad Sci USA. 1996; 93:2774-2778
153. Sanghera J S, Weinstein S L, Aluwalia M, Girn J, Pelech S L. Activation of multiple proline-directed kinases by bacterial lipopolysaccharide in murine macrophages. J Immunol. 1996; 156:4457-4465
154. Feingold K, Kim M S, Shigenaga J, Moser A, Grunfeld C. Altered expression of nuclear hormone receptors and coactivators in mouse heart during the acute-phase response. Am J Physiol Endocrinol Metab. 2004; 286:E201-207
155. Finck B N, Lehman J J, Leone T C, Welch M J, Bennett M J, Kovacs A, Han X, Gross R W, Kozak R, Lopaschuk G D, Kelly D P. The cardiac phenotype induced by pparalpha overexpression mimics that caused by diabetes mellitus. J Clin Invest. 2002; 109:121-130
156. Finck B N, Bernal-Mizrachi C, Han D H, Coleman T, Sambandam N, LaRiviere L L, Holloszy J O, Semenkovich C F, Kelly D P. A potential link between muscle peroxisome proliferator-activated receptor-alpha signaling and obesity-related diabetes. Cell Metab. 2005; 1:133-144
157. Cheng L, Ding G, Qin Q, Huang Y, Lewis W, He N, Evans R M, Schneider M D, Brako F A, Xiao Y, Chen Y E, Yang Q. Cardiomyocyte-restricted peroxisome proliferator-activated receptor-delta deletion perturbs myocardial fatty acid oxidation and leads to cardiomyopathy. Nat Med. 2004; 10:1245-1250
158. Cha B S, Ciaraldi T P, Park K S, Carter L, Mudaliar S R, Henry R R. Impaired fatty acid metabolism in type 2 diabetic skeletal muscle cells is reversed by ppargamma agonists. Am J Physiol Endocrinol Metab. 2005; 289:E151-159
159. Son N H, Yu S, Tuinei J, Arai K, Hamai H, Homma S, Shulman G I, Abel E D, Goldberg I J. Ppargamma-induced cardiolipotoxicity in mice is ameliorated by pparalpha deficiency despite increases in fatty acid oxidation. J Clin Invest. 2010; 120:3443-3454
160. Angus D C, Linde-Zwirble W T, Lidicker J, Clermont G, Carcillo J, Pinsky M R. Epidemiology of severe sepsis in the united states: Analysis of incidence, outcome, and associated costs of care. Crit Care Med. 2001; 29:1303-1310
161. Ballard F B, Danforth W H, Naegle S, Bing R J. Myocardial metabolism of fatty acids. J Clin Invest. 1960; 39:717-723
162. Neubauer S. The failing heart—an engine out of fuel. N Engl J Med. 2007; 356:1140-1151
163. Osorio J C, Stanley W C, Linke A, Castellari M, Diep Q N, Panchal A R, Hintze T H, Lopaschuk G D, Recchia F A. Impaired myocardial fatty acid oxidation and reduced protein expression of retinoid×receptor-alpha in pacing-induced heart failure. Circulation. 2002; 106:606-612
164. Tessier J P, Thurner B, Jungling E, Luckhoff A, Fischer Y. Impairment of glucose metabolism in hearts from rats treated with endotoxin. Cardiovasc Res. 2003; 60:119-130
165. Nemoto S, Vallejo J G, Knuefermann P, Misra A, Defreitas G, Carabello B A, Mann D L. Escherichia coli 1 ps-induced lv dysfunction: Role of toll-like receptor-4 in the adult heart. Am J Physiol Heart Circ Physiol. 2002; 282:H2316-2323
166. Michel J B, Lattion A L, Salzmann J L, Cerol M L, Philippe M, Camilleri J P, Corvol P. Hormonal and cardiac effects of converting enzyme inhibition in rat myocardial infarction. Circ Res. 1988; 62:641-650
167. Ng W A, Grupp I L, Subramaniam A, Robbins J. Cardiac myosin heavy chain mrna expression and myocardial function in the mouse heart. Circ Res. 1991; 68:1742-1750
168. Son N H, Park T S, Yamashita H, Yokoyama M, Huggins L A, Okajima K, Homma S, Szabolcs M J, Huang L S, Goldberg I J. Cardiomyocyte expression of ppargamma leads to cardiac dysfunction in mice. J Clin Invest. 2007; 117:2791-2801
169. Wu Z, Puigserver P, Andersson U, Zhang C, Adelmant G, Mootha V, Troy A, Cinti S, Lowell B, Scarpulla R C, Spiegelman B M. Mechanisms controlling mitochondrial biogenesis and respiration through the thermogenic coactivator pgc-1. Cell. 1999; 98:115-124
170. Vega R B, Huss J M, Kelly D P. The coactivator pgc-1 cooperates with peroxisome proliferator-activated receptor alpha in transcriptional control of nuclear genes encoding mitochondrial fatty acid oxidation enzymes. Mol Cell Biol. 2000; 20:1868-1876
171. Lee W J, Kim M, Park H S, Kim H S, Jeon M J, Oh K S, Koh E H, Won J C, Kim M S, Oh G T, Yoon M, Lee K U, Park J Y. Ampk activation increases fatty acid oxidation in skeletal muscle by activating pparalpha and pgc-1. Biochem Biophys Res Commun. 2006; 340:291-295
172. Kehrer J P, Biswal S S, La E, Thuillier P, Datta K, Fischer S M, Vanden Heuvel J P. Inhibition of peroxisome-proliferator-activated receptor (ppar)alpha by mk886. Biochem J. 2001; 356:899-906
173. Drosatos K, Bharadwaj K G, Lymperopoulos A, Ikeda S, Khan R, Hu Y, Agarwal R, Yu S, Jiang H, Steinberg S F, Blaner W S, Koch W J, Goldberg I J. Cardiomyocyte lipids impair {beta} {beta}-adrenergic receptor function via pkc activation. Am J Physiol Endocrinol Metab. 2010
174. Iliopoulos D, Drosatos K, Hiyama Y, Goldberg I J, Zannis V I. Microrna-370 controls the expression of microrna-122 and cptlalpha and affects lipid metabolism. J Lipid Res. 2010; 51:1513-1523
175. Drosatos K, Kypreos K E, Zannis V I. Residues leu261, trp264, and phe265 account for apolipoprotein e-induced dyslipidemia and affect the formation of apolipoprotein e-containing high-density lipoprotein. Biochemistry. 2007; 46:9645-9653
176. Drosatos K, Sanoudou D, Kypreos K E, Kardassis D, Zannis V I. A dominant negative form of the transcription factor c-jun affects genes that have opposing effects on lipid homeostasis in mice. J Biol Chem. 2007; 282:19556-19564
177. Hovsepian E, Penas F, Goren N B. 15-deoxy-delta12, 14 prostaglandin gj2 but not rosiglitazone regulates metalloproteinase 9, nos-2, and cyclooxygenase 2 expression and functions by peroxisome proliferator-activated receptor gamma-dependent and -independent mechanisms in cardiac cells. Shock. 2010; 34:60-67
178. Ding G, Fu M, Qin Q, Lewis W, Kim H W, Fukai T, Bacanamwo M, Chen Y E, Schneider M D, Mangelsdorf D J, Evans R M, Yang Q. Cardiac peroxisome proliferator-activated receptor gamma is essential in protecting cardiomyocytes from oxidative damage. Cardiovasc Res. 2007; 76:269-279
179. Uz T, Dimitrijevic N, Imbesi M, Manev H, Manev R. Effects of mk-886, a 5-lipoxygenase activating protein (flap) inhibitor, and 5-lipoxygenase deficiency on the forced swimming behavior of mice. Neurosci Lett. 2008; 436:269-272
180. Voelkel N F, Tuder R M, Wade K, Hoper M, Lepley R A, Goulet J L, Koller B H, Fitzpatrick F. Inhibition of 5-lipoxygenase-activating protein (flap) reduces pulmonary vascular reactivity and pulmonary hypertension in hypoxic rats. J Clin Invest. 1996; 97:2491-2498
181. Mazzola C, Medalie J, Scherma M, Panlilio L V, Solinas M, Tanda G, Drago F, Cadet J L, Goldberg S R, Yasar S. Fatty acid amide hydrolase (faah) inhibition enhances memory acquisition through activation of ppar-alpha nuclear receptors. Learn Mem. 2009; 16:332-337
182. Aviello G, Matias I, Capasso R, Petrosino S, Borrelli F, Orlando P, Romano B, Capasso F, Di Marzo V, Izzo A A. Inhibitory effect of the anorexic compound oleoylethanolamide on gastric emptying in control and overweight mice. J Mol Med. 2008; 86:413-422
183. Melis M, Pillolla G, Luchicchi A, Muntoni A L, Yasar S, Goldberg S R, Pistis M. Endogenous fatty acid ethanolamides suppress nicotine-induced activation of mesolimbic dopamine neurons through nuclear receptors. J Neurosci. 2008; 28:13985-13994
184. Cuzzocrea S, Pisano B, Dugo L, Ianaro A, Maffia P, Patel N S, Di Paola R, Ialenti A, Genovese T, Chatterjee P K, Di Rosa M, Caputi A P, Thiemermann C. Rosiglitazone, a ligand of the peroxisome proliferator-activated receptor-gamma, reduces acute inflammation. Eur J Pharmacol. 2004; 483:79-93
185. Stavinoha M A, Rayspellicy J W, Hart-Sailors M L, Mersmann H J, Bray M S, Young M E. Diurnal variations in the responsiveness of cardiac and skeletal muscle to fatty acids. Am J Physiol Endocrinol Metab. 2004; 287:E878-887
186. Cook S A, Sugden P H, Clerk A. Activation of c-jun n-terminal kinases and p38-mitogen-activated protein kinases in human heart failure secondary to ischaemic heart disease. J Mol Cell Cardiol. 1999; 31:1429-1434
187. Haq S, Choukroun G, Lim H, Tymitz K M, del Monte F, Gwathmey J, Grazette L, Michael A, Hajjar R, Force T, Molkentin J D. Differential activation of signal transduction pathways in human hearts with hypertrophy versus advanced heart failure. Circulation. 2001; 103:670-677
188. Bone R C, Balk R A, Cerra F B, Dellinger R P, Fein A M, Knaus W A, Schein R M, Sibbald W J. Definitions for sepsis and organ failure and guidelines for the use of innovative therapies in sepsis. The accp/sccm consensus conference committee. American college of chest physicians/society of critical care medicine. Chest. 1992; 101:1644-1655
189. Angus D C, Linde-Zwirble W T, Lidicker J, Clermont G, Carcillo J, Pinsky M R. Epidemiology of severe sepsis in the united states: Analysis of incidence, outcome, and associated costs of care. Crit Care Med. 2001; 29:1303-1310
190. Wilson T M, Brown P J, Sternbach, D D, Henke B R. The PPARs: from orphan receptors to drug discovery. J. Med. Chem., 2000, 43(4), 527-550
191. Berger, J, Moller D E. The Mechanism of Action of PPARs. Annu. Rev. Med., 2002, 53, 409-435
192. Wilson, T M, Brown P J, Sternbach, D D, Henke B R. The PPARs: from orphan receptors to drug discovery. J. Med. Chem., 2000, 43(4), 527-550
193. Kliewer, S A, Xu H E, Lamber M H, Willson T M. Peroxisome proliferator-activated receptors: from genes to physiology Recent Prog Horm Res., 2001, 56, 239-63
194. Moller, D E and Berger, J P, Role of PPARs in the regulation of obesity-related insulin sensitivity and inflammation. Int J Obes Relat Metab Disord., 2003, 27 Suppl 3, S 17-21
195. Ram, V J, Therapeutic Significance of peroxisome proliferative-activated receptor modulators in diabetes. Drugs Today, 2003, 39(8), 609-32)
196. Motojima K., Peroxisome proliferator-activated receptor (PPAR): structure, mechanisms of activation and diverse functions: Cell Struct Funct., 1993, 18(5), 267-77)
197. Barger P M, Kelly D P. PPAR Signaling in the control of cardiac energy metabolism. Trends Cardiovasc. Med., 2000, 10 (6), 238-245)
198. Lehmann, J M, Moore L B, Smith-Oliver T A, Wilkison W O, Willson T M, Kliener, S A. An antidiabetic thiazolidinedione is a high affinity ligand for peroxisome proliferator-activated receptor gamma (PPAR gamma), J. Biol. Chem., 1995 Jun. 2; 270, 12953-12956, 1995)
199. Clark et al., U.S. Pat. No. 7,371,777
200. Drosatos K, Drosatos-Tampakaki Z, Khan R, Homma S, Schulze P C, Zannis V I, Goldberg I J. Inhibition of c-jun-n-terminal kinase increases cardiac ppar{alpha} expression and fatty acid oxidation and prevents 1 ps-induced heart dysfunction. J Biol Chem. 2011 Oct. 21; 286 (42): 36331-9
201. Feingold K, Kim M S, Shigenaga J, Moser A, Grunfeld C. Altered expression of nuclear hormone receptors and coactivators in mouse heart during the acute-phase response. Am J Physiol Endocrinol Metab. 2004; 286:E201-207
202. Maitra U, Chang S, Singh N, Li L. Molecular mechanism underlying the suppression of lipid oxidation during endotoxemia. Mol Immunol. 2009; 47:420-425
203. Bagby G J, Spitzer J A. Lipoprotein lipase activity in rat heart and adipose tissue during endotoxic shock. Am J Physiol. 1980; 238:H325-330
204. Lu B, Moser A, Shigenaga J K, Grunfeld C, Feingold K R. The acute phase response stimulates the expression of angiopoietin like protein 4. Biochem Biophys Res Commun. 2010; 391:1737-1741
205. Son N H, Yu S, Tuinei J, Arai K, Hamai H, Homma S, Shulman G I, Abel E D, Goldberg I J. Ppargamma-induced cardiolipotoxicity in mice is ameliorated by pparalpha deficiency despite increases in fatty acid oxidation. J Clin Invest. 2010; 120:3443-3454
206. Son N H, Park T S, Yamashita H, Yokoyama M, Huggins L A, Okajima K, Homma S, Szabolcs M J, Huang L S, Goldberg I J. Cardiomyocyte expression of ppargamma leads to cardiac dysfunction in mice. J Clin Invest. 2007; 117:2791-2801
207. Carlson D L, Willis M S, White D J, Horton J W, Giroir B P. Tumor necrosis factor-alpha-induced caspase activation mediates endotoxin-related cardiac dysfunction. Crit Care Med. 2005; 33:1021-1028
208. Fallach R, Shainberg A, Avlas O, Fainblut M, Chepurko Y, Porat E, Hochhauser E. Cardiomyocyte toll-like receptor 4 is involved in heart dysfunction following septic shock or myocardial ischemia. J Mol Cell Cardiol. 2010; 48:1236-1244
209. Knuefermann P, Nemoto S, Misra A, Nozaki N, Defreitas G, Goyert S M, Carabello B A, Mann D L, Vallejo J G. Cd14-deficient mice are protected against lipopolysaccharide-induced cardiac inflammation and left ventricular dysfunction. Circulation. 2002; 106:2608-2615
210. Ohlsson K, Bjork P, Bergenfeldt M, Hageman R, Thompson R C. Interleukin-1 receptor antagonist reduces mortality from endotoxin shock. Nature. 1990; 348:550-552
211. Agrawal, S, and Zhao, Q. Antisense therapeutics (1998) Curr. Opi. Chemical Biol. Vol. 2, 519-528
212. Agrawal, S. and Zhang, R. (1997) Pharmacokinetics of oligonucleotides, CIBA Found. Symp. Vol. 209, 60-78
213. Zhao, Q, et al., (1998), Cellular Distribution of Phosphorothioate Oligonucleotide Following Intravenous Administration in Mice, Antisense Nucleic Acid Drug Dev. Vol 8, 451-458
214. Anderson, K. P., et al., (1996) Inhibition of human cytomegalovirus immediate-early gene expression by an antisense oligonucleotide complementary to immediate-early RNA Antimicrobial Agents Chemother. Vol. 40 (9), 2004-2011
215. Borchers, et al., U.S. Pat. No. 6,828,151
216. Zhang W, et al., MicroRNA-520b Inhibits Growth of Hepatoma Cells by Targeting MEKK2 and Cyclin D1, February 2012 Volume 7 Issue 2 e31450

What is claimed is:

1. A method for increasing or maintaining cardiac function in a subject, comprising administering therapeutically effective amounts of an agent that increases fatty acid oxidation in the heart.

2. The method of claim 1, wherein the subject has sepsis or is at risk of developing sepsis.

3. The method of claim 1, wherein the subject has heart failure or is at risk of developing heart failure.

4. The method of claim 1, wherein the agent is selected from the group comprising PPARα agonists, PPARγ agonists, dual PPARα and PPARγ agonists, or combinations thereof.

5. The method of claim 1, wherein the agent inhibits JNK 1 or JNK 2 or both.

6. The method of claim 5 wherein the JNK inhibitor is SP600125.

7. The method of claim 5, wherein the JNK inhibitor is an inhibitory RNA selected from the group comprising an antisense nucleic acid, siRNA micro RNA (miRNA), short hairpin RNA, ribozyme, microRNA mimic, supermir, and aptamer that specifically hybridizes to JNK thereby reducing its expression.

8. The method of claim 4, wherein the agent is PPARα-coactivator-1 (PGC-1), estrogen-related receptor (ERR)α, or a combination thereof.

9. The method of claim 4, wherein the PPARα agonist is selected from the group comprising Alpha WY-14643 GW9578, GW-590735, K-111, LY-674, KRP-101, DRF-10945, LY518674, Propanoic Acid 2-[4-[3-[2,5-dihydro-1-[(4-methylphenyl)methyl]-5-oxo-1H-1,2,4-triazol-3-yl]propyl]phenoxy]-2-methyl, fibrate, fenofibrate, clofibrate, and bezafibrate.

10. The method of claim 4, wherein the PPARγ agonist is selected from the group comprising thiazolidinedione, rosiglitazone, pioglitazone, MCC-555, GL-262570, englitazone, darglitazone, isaglitazone, JTT-501, T-895645, R-119702, N,N-2344, YM-440, GI 262570, R-483 and rivoglitazone.

11. A pharmaceutical composition comprising therapeutically effective amounts of one member from at least two of the following groups: (i) JNK1 or a JNK2 inhibitors including antisense nucleic acids, siRNAs, shRNAs, microRNAs (miRNA), ribozymes, microRNA mimics, supermirs, and aptamers; (ii) a PPAR agonists selected from the group comprising PPAR agonists, PPARγ agonists, dual PPARα and PPAR γ agonists; and (iii) PPARα-co-activator-1 (PGC-1) and (iii) estrogen-related receptor (ERR), which amounts treat or prevent cardiac dysfunction in a subject having heart failure or sepsis or at risk of developing them; or in amounts that increase cardiac function also in the patient having heart failure or sepsis or at risk of developing them.

12. A kit comprising the pharmaceutical composition of claim 11.

13. A method for treating or preventing cardiac dysfunction in a subject having sepsis or at risk of developing sepsis, comprising administering therapeutically effective amounts of an agent that increases fatty acid oxidation in the heart.

14. The method of claim 1 wherein the agent is administered before cardiac function is diminished, or significantly reduced.

15. The method of claim 1, wherein the agent comprises at least two members selected from the group comprising JNK inhibitors, PPARα agonists, PPARγ agonists, dual PPARα and PPARγ agonists, or combinations thereof, or PPARα-coactivator-1 (PGC-1), estrogen-related receptor (ERR)α, or a combination thereof.

16. The method of claim 1, wherein the therapeutically effective amount of the agent is administered within 24 hours of a diagnosis of sepsis or at a risk of developing sepsis.

17. The method of claim 13, wherein the therapeutically effective amount of the agent is administered within 24 hours of a diagnosis of sepsis or at a risk of developing sepsis.

18. A method for treating or preventing cardiac dysfunction in a subject having heart failure comprising administering therapeutically effective amounts of an agent that increases fatty acid oxidation in the heart.

19. The method of claim 18, wherein the therapeutically effective amount of the agent is administered within 24 hours of a diagnosis of sepsis or at a risk of developing sepsis.
